(19) United States
(12) Patent Application Publication
(10) Pub. No.: US 2019/0211098 A1
(43) Pub. Date: Jul. 11, 2019

(54) USE OF PILRA BINDING AGENTS FOR TREATMENT OF A DISEASE

(71) Applicant: Genentech, Inc., South San Francisco, CA (US)

(72) Inventors: Tushar R. Bhangale, San Francisco, CA (US); Robert R. Graham, San Bruno, CA (US); David V. Hansen, San Mateo, CA (US); Nisha Rathore, Burlingame, CA (US); Ali A. Zarring, Brisbane, CA (US); Jack J. Bevers, III, South San Francisco, CA (US); Jianhuan Zhang, South San Francisco, CA (US)

(73) Assignee: Genentech, Inc., South San Francisco, CA (US)

(21) Appl. No.: 16/227,923

(22) Filed: Dec. 20, 2018

Related U.S. Application Data
(60) Provisional application No. 62/609,852, filed on Dec. 22, 2017.

(51) Int. Cl. C07K 16/28

(52) U.S. Cl. CPC C07K 16/2803; C07K 2317/92; C07K 2317/34; C07K 2317/76

(57) ABSTRACT

Provided herein are methods of treating a subject, methods of predicting the response of a subject and selecting a subject suffering from a disease associated with myeloid cell dysfunction. In particular, provided herein are methods for treatment or diagnosis of a disease associated with myeloid cell dysfunction, such as Alzheimer's Disease (AD) and Herpes Simplex Virus-1 (HSV-1) infection, with an agent specifically binding to Paired Immunoglobulin-like Type 2 Receptor Alpha (PILRA), such as an antibody as well as pharmaceutical formulations comprising the same.

### CROSS-REFERENCE TO RELATED APPLICATIONS

This application claims priority to U.S. Provisional Patent Application Ser. No. 62/609,852 filed on Dec. 22, 2017, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Dec. 19, 2018, is named P34607-US-1 SL.txt and is 149,569 bytes in size.

### TECHNICAL FIELD

Provided herein are methods of treating a subject, methods of predicting the response of a subject and selecting a subject suffering from a disease associated with myeloid cell dysfunction. In particular, provided herein are methods for treatment or diagnosis of a disease associated with myeloid cell dysfunction, such as Alzheimer's Disease (AD) and Herpes Simplex Virus-1 (HSV-1) infection, with an agent specifically binding to Paired Immunoglobulin-like Type 2 Receptor Alpha (PILRA), such as an antibody as well as pharmaceutical formulations comprising the same.

### BACKGROUND

AD results from a complex interaction of environmental and genetic risk factors (see, e.g., Holtzman et al., Sci. Transl. Med., 3 (77):77sr1 (2011)). Proposed environmental risk factors include a history of head trauma (see, e.g., O'Meara et al., Am. J. Epidemiol. 146, 373-84 (1997)) and infection (see, e.g., Harris et al., J. Alzheimers. Dis. 48, 319-53 (2015)). In recent years, large-scale genome-wide association studies (GWAS) and family-based studies have made considerable progress in defining the genetic component of AD-risk and >20 AD-risk loci have been identified (see, e.g., Lambert et al., Nat. Genet. 45, 1452-8 (2013)). A key role for microglial/monocyte biology in modulating risk of AD has emerged from the analysis of the loci associated with AD-risk.

A role for infection in accelerating AD has been described (see, e.g., Alam et al., Curr. Top. Med. Chem. 17, 1390-1399 (2017)). Chronic infections, including HSV-1, have been linked to AD. HSV-1 is a neurotropic virus that infects a large fraction of the adult population and has frequent reactivation events. HSV-1 acute encephalitis preferentially targets regions affected in AD. Furthermore, studies have reported elevated HSV-1 titers in AD cases and that high avidity HSV-1 antibodies correlate with protection from cognitive decline (see, e.g., Agostini et al., Brain. Behav. Immun. 58, 254-260 (2016)).

HSV-1 is a member of the alpha herpes virus subfamily and can cause recurrent mucocutaneous lesions on the mouth, face, or genitalia and potentially meningitis or encephalitis. HSV-1 glycoprotein B (gB) is a ligand for PILRA (see, e.g., Satoh et al., Cell 132:935-944 (2008)). Interestingly, expression of PILRA on cells enhances HSV-1 entry, whereas expression of Paired Immunoglobulin-like Type 2 Receptor Beta (PILRB) does not (see, e.g., Fan and Longnecker, J. Virol. 84(17):8664-8672 (2010)). Interestingly, binding of PILRA to HSV-1 gB also requires sialylated O-glycans (T53, T480) (see, e.g., Fan et al., J. Virol. 83(15):7384-7390 (2009)). PILRA specifically associates with HSV-1 gB, but not with other HSV-1 glycoproteins, although some other envelope proteins are known to be O-glycosylated (see, e.g., Fan et al., J. Virol. 83(15):7384-7390 (2009)).

Both, PILRA and PILRB are expressed as monomeric transmembrane proteins with a single V-set Ig-like extracellular domain (see, e.g., Lu et al., PNAS 111, 8221-8226 (2014)). PILRA is considered a cell surface inhibitory receptor that recognizes specific O-glycosylated proteins and is expressed on various innate immune cell types including microglia. Furthermore, PILRA is capable of binding O-glycoslated proteins containing a consensus amino acid motif.

The data presented in the present application suggest that PILRA ligand binding plays a role in the pathogenesis of AD. There is currently no treatment that halts or significantly slows the progression of AD, creating an unmet need for patients with AD. Thus, there is a need to identify efficacious therapies for AD and improved methods for understanding how to treat AD patients. Specifically, diagnostic methods useful for identifying patients at risk for AD and patients likely to benefit from treatments with anti-PILRA agents would greatly benefit clinical management of these patients.

All references cited herein, including patent applications and publications, are incorporated by reference in their entirety.

### SUMMARY

Provided herein are methods for treating a disease associated with myeloid cell dysfunction in a subject comprising administering an effective amount of an agent to the subject, wherein the agent specifically binds to one or more variants of PILRA thereby inhibiting the interaction between PILRA and any one of its ligands.

Further provided herein are methods of selecting a subject having a disease associated with myeloid cell dysfunction for a treatment with an agent inhibiting the interaction between one or more variants of PILRA and any one of its ligands, comprising determining the presence or absence of the one or more variants of PILRA in a biological sample from the subject, wherein the presence of the one or more variants of PILRA indicates that the subject is suitable for treatment with the agent.

Further provided herein are methods of predicting the response of a subject having a disease associated with myeloid cell dysfunction to a treatment with an agent specifically binding to one or more variants of PILRA, the method comprising (a) measuring whether the agent specifically binding to the one or more variants of PILRA inhibits the interaction between PILRA and any one of its ligands as compared to a reference level, and (d) predicting that the subject will respond to the treatment when the interaction between PILRA and any one of its ligands is inhibited as compared to the reference level and predicting that the subject will not respond to the treatment when the interaction between PILRA and any one of its ligands is not inhibited as compared to the reference level.

Further provided herein are methods for detecting the presence or absence of one or more variants of PILRA indicating that a subject having a disease associated with myeloid cell dysfunction is suitable for treatment with an agent inhibiting the interaction between PILRA and any one of its ligands, comprising (a) contacting a sample from the subject with a reagent capable of detecting the presence or absence of the one more variants of PILRA; and (b) determining the presence or absence of the one or more variants of PILRA, wherein the presence of the one or more variants of PILRA indicates that the subject is suitable for treatment with an agent inhibiting the interaction between PILRA and any one of its ligands.

Further provided herein are methods for selecting an agent for treating a disease associated with myeloid cell dysfunction, comprising determining whether the agent inhibits the interaction between PILRA and any one of its ligands, wherein the agent that inhibits the interaction between PILRA and any one of its ligands is suitable for treating the disease associated with myeloid cell dysfunction.

In some embodiments of any of the methods, the disease associated with myeloid cell dysfunction is selected from the group consisting of AD and HSV-1 infection. In some embodiments of any of the methods, the myeloid cell dysfunction is associated with a decreased myeloid cell activity.

In some embodiments of any of the methods, the one or more variants of PILRA are encoded by a polynucleotide sequence comprising one or more SNPs. In some embodiments, the one or more SNPs result in one or a combination of the following amino acids at the given positions i) the amino acid glycine (G78) or arginine (R78) at position 78; ii) the amino acid serine (S279) or leucine (L279) at position 279; of the full-length unprocessed PILRA (SEQ ID NO:01 -SEQ ID NO:03). In some embodiments, the SNP results in the amino acid arginine at position 78 of the full-length unprocessed PILRA (SEQ ID NO:01 -SEQ ID NO:03). In some embodiments, the SNP is rs1859788.

In some embodiments of any of the methods, the agent stabilizes the non-ligand bound form of the PILRA receptor. In some embodiments of any of the methods, the agent reduces the inhibitory signaling in myeloid cells. In some embodiments of any of the methods, the agent inhibits the interaction between PILRA and any one of its ligands by binding to one or more amino acids on PILRA. In some embodiments, the one or more amino acids are located within the sialic acid (SA) binding region of PILRA. In some embodiments, the one or more amino acids are selected from the group consisting of Y33, R126, T131, R132, Q138, W139 and Q140 of the full-length unprocessed PILRA (SEQ ID NO:01 -SEQ ID NO:03). In some embodiments, the one or more amino acids are R126 and/or Q140 of the full-length unprocessed PILRA (SEQ ID NO:01-SEQ ID NO:03).

In some embodiments of any of the methods, the agent inhibits the interaction between PILRA and any one of its ligands by at least 50% as compared to a reference level. In some embodiments, the reference level is based on the interaction between the G78 variant of PILRA and any one of its ligands.

In some embodiments of any of the methods, the agent decreases infection of a myeloid cell during HSV-1 recurrence.

In some embodiments of any of the methods, the myeloid cell is a CNS resident myeloid cell. In some embodiments, the CNS resident myeloid cell is selected from the group consisting of microglia, perivascular macrophages, meningeal macrophages, and choroid plexus macrophages. In some embodiments, the CNS resident myeloid cell is a microglia.

In some embodiments of any of the methods, the agent is selected from the group consisting of an antibody, a polypeptide, a polynucleotide and a small molecule. In some embodiments of any of the methods, the agent is an antibody. In some embodiments, the antibody is a monoclonal antibody. In some embodiments, the monoclonal antibody is a human, humanized, or chimeric antibody. In some embodiments, the antibody is a full length IgG1 antibody.

In some embodiments of any of the methods, the ligand is an endogenous ligand. In some embodiments, the endogenous ligand is selected from the group consisting of APLP1, C16orf54, C4A, C4B, CLEC4G, COLEC12, DAG1, EVA1C, FceRII, IL17RA, LILRB5, LRRC15, LRRTM4, NPDC1, PIANP, and PRSS55. In some embodiments of any of the methods, the ligand is an exogenous ligand. In some embodiments, the exogenous ligand is HSV-1 glycoprotein B.

In some embodiments of any of the methods, the ligand is an endogenous ligand. In some embodiments, the endogenous ligand is selected from the group consisting of APLP1, C16orf54, C4A, C4B, CD99, CLEC4G, COLEC12, DAG1, EVA1C, FceRII, IL17RA, LILRB5, LRRC15, LRRTM4, NPDC1, PIANP, and PRSS55. In some embodiments of any of the methods, the ligand is an exogenous ligand. In some embodiments, the exogenous ligand is HSV-1 glycoprotein B.

In some embodiments of any of the methods, the sample is selected from the group consisting of cerebrospinal fluid, blood, serum, sputum, saliva, mucosal scraping, tissue biopsy, lacrimal secretion, semen, and sweat. In some embodiments of any of the methods, the subject is a human.

Further provided herein is an agent specifically binding to one or more variants of PILRA for use in medical treatment or diagnosis including therapy and/or treating of a disease associated with myeloid cell dysfunction. In some embodiments, the agent stabilizes the non-ligand bound form of the PILRA receptor. In some embodiments of any of the agents, the agent reduces the inhibitory signaling in myeloid cells. In some embodiments, the agent inhibits the interaction between the one or more variants of PILRA and any one of its ligands by binding to one or more amino acids on PILRA. In some embodiments, the one or more amino acids are located within the SA binding region of PILRA. In some embodiments, the one or more amino acids are selected from the group consisting of Y33, R126, T131, R132, Q138, W139 and Q140 of the full-length unprocessed PILRA (SEQ ID NO:01 -SEQ ID NO:03). In some embodiments, the one or more amino acids are R126 and/or Q140 of the full-length unprocessed PILRA (SEQ ID NO:01-SEQ ID NO:03). In some embodiments, the agent inhibits the interaction between the one or more variants of PILRA and any one of its ligands by at least 50% as compared to a reference level. In some embodiments, the reference level is based on the interaction between the G78 variant of PILRA and any one of its ligands. In some embodiments, the agent decreases infection of a myeloid cell during HSV-1 recurrence.

In some embodiments of any of the agents, the myeloid cell is a CNS resident myeloid cell. In some embodiments, the CNS resident myeloid cell is selected from the group consisting of microglia, perivascular macrophages, meningeal macrophages, and choroid plexus macrophages. In some embodiments, the CNS resident myeloid cell is a microglia.

In some embodiments, the agent is selected from the group consisting of an antibody, a polypeptide, a polynucleotide and a small molecule. In some embodiments, the agent is an antibody. In some embodiments, the antibody is a monoclonal antibody. In some embodiments, the monoclonal antibody is a human, humanized, or chimeric antibody. In some embodiments, the antibody is a full length IgG1 antibody.

In some embodiments, the disease associated with myeloid cell dysfunction is selected from the group consisting of AD and HSV-1 infection.

Further provided herein is a pharmaceutical formulation comprising a pharmaceutically active amount of an agent specifically binding to one or more variants of PILRA as described herein and a pharmaceutically acceptable carrier.

### BRIEF DESCRIPTION OF THE DRAWINGS

FIG. 1A-G show the association of the PILRA rs1859788 SNP encoding the R78 variant of PILRA (AD-protective) and PILRA variants including the R78 variant with reduced ligand binding. Statistical analysis of ligand binding experiments is two-tailed unpaired t-test (p values <0.05=*, <0.005=**, <0.0005=***, <0.0001=****) on 3-4 independent experiments.

FIG. 1A: Shows the association of variants in the 7q21 locus with AD-risk in the IGAP phase 1 dataset.

FIG. 1B: Schematic diagram depicting the ectopic expression of PILRA as a membrane protein in 293T cells, and application of soluble PILRA ligands (in this case, NPDC1 fused to mFC, a murine IgG2a fragment) to assess PILRA-ligand interactions.

FIG. 1C: 293T cells were transfected with an empty vector, G78 variant of PILRA (AJ400841), one of two synthetic mutations previously predicted to impair PILRA ligand binding (A72 and A76 variant), a synthetic mutation outside the SA binding domain (G80 variant), and the R78 variant of PILRA (AD-protective). The binding of NPDC1-mFC to different PILRA variant-transfected cells was measured by flow cytometry. The percent of cells expressing PILRA and positive for NPDC1 is indicated in each panel.

FIG. 1D: Schematic diagram depicting the ectopic expression of PILRA ligands (in this case, NPDC1, HSV-1 gB, or PIANP) as membrane-associated proteins in 293T cells, and application of soluble PILRA variants (in the form of the PILRA extracellular domain fused to mFC) to assess PILRA-ligand interactions.

FIG. 1E: 293T cells were transfected with the ligand NPDC1. Binding of different PILRA variants to ligand-transfected cells is shown as percentage of MFI of PILRA-mFC binding considering the binding of the G78 variant of PILRA as 100% for each experiment.

FIG. 1F: 293T cells were transfected with the ligand HSV-1 gB. Binding of different PILRA variants to ligand-transfected cells is shown as percentage of MFI of PILRA-mFC binding considering the binding of the G78 variant of PILRA as 100% for each experiment.

FIG. 1G: 293T cells were transfected with the ligand myc-PIANP. Binding of different PILRA variants to ligand-transfected cells is shown as percentage of MFI of PILRA-mFC binding considering the binding of the G78 variant of PILRA as 100% for each experiment.

FIG. 2A-E show the structural determinants of PILRA in apo- (ligand-free), and ligand-bound conformations, and conformational changes in R78 variant of PILRA that reduce ligand binding.

FIG. 2A: The unliganded crystal structure of R78 PILRA displays an “open” conformation with an unformed SA binding region, where the essential R126 side-chain remains in an extended conformation incompatible with SA-binding. The R78 side chain hydrogen bonds to the Q140 side chain directly, reducing its availability to interact with R126. The R78-Q140 interaction also sterically occludes F76 from moving into a ligand-binding competent position, likely alters the dynamics of the CC′ loop, and therefore serves to help stabilize the “open” or apo-state of PILRA.

FIG. 2B: The apo-crystal structure of the G78 variant of PILRA reveals a similar overall conformation with the apo-R78 structure, but the Q140-R126 interaction network remains pre-formed and the “downward” movement of F76 is not impeded in the absence of the R78 side chain.

FIG. 2C: The structure of the sialylated 0-linked sugar T antigen sTn-bound G78 variant of PILRA reveals the concerted ligand-induced conformational changes across the receptor that lead to simultaneous engagement of the SA-motif by direct coordination to R126 and the critical involvement of F76 in peptide-ligand recognition. Notably, the ligand-bound conformation of R78 is expected to be highly similar, as the arginine side chain of the R78 variant of PILRA would be predicted to point towards solvent and have little direct consequence in the bound conformation. For completeness, aromatic residues including Y33 and W59 at the bottom of the receptor also undergo significant ligand-induced conformational changes upon ligand binding to form a portion of the sugar-binding site.

FIG. 2D: 293T cells were transfected with G78 variant of PILRA, a synthetic mutant predicted to bring conformational changes in PILRA (Q140A, referred to as A140 in the figure), a synthetic mutant predicted non-essential for conformation changes (S141A, referred to as A141 in the figure), and the R78 variant of PILRA. The binding of NPDC1-mFC to different PILRA variant-transfected cells was measured by flow cytometry. The percent of cells expressing PILRA and positive for NPDC1 is indicated in each panel. Statistical analysis is two-tailed unpaired t-test (p values <0.05=*, <0.005=**, <0.0005=***, <0.0001=****) on three independent experiments.

FIG. 2E: PILRA-mFc (G78, R78, or A140 variants) were immobilized on a ProteOn GLC sensor chip. NPDC1.mFc or control mFc diluted in PBST were injected over the immobilized PILRA proteins. NPDC1-mFc bound to the G78 variant of PILRA (AD-risk) to a greater extent as compared to the R78 variant (AD-protective) and A140 (mutation of essential residue for conformational change to form SA binding region).

FIG. 3A-E shows that PILRA R78 reduces the entry of HSV-1 into human monocyte-differentiated macrophages (hMDM). hMDMs derived from five pairs of healthy, PILRA-genotyped human donors and were infected with 0.01, 0.1, 1, and 10 multiplicities of infection (MOI) of HSV-1 virus for 6, 18, and 36 hrs. Statistical analysis is two-tailed paired or unpaired t-test (p values <0.05=*, <0.005=**, <0.0005=***, <0.0001=****) performed on 3-5 genotyped individual donor pairs.

FIG. 3A: Representative images of cells infected with 0.1 MOI for 18 hrs. hMDMs from PILRA R78 donors have less cytopathic effect compared to G78 donors (see arrows).

FIG. 3B: LDH cytotoxicity assay was performed on supernatants harvested from HSV-1-infected hMDMs after 18 hrs. Results are % cytotoxicity—amount of LDH in culture supernatant after infection compared to LDH released from cells completely lysed by lysis buffer, with completely lysed cells (maximum LDH release) considered as 100% for each donor. Each shape represents one donor pair. Homozygous R78 hMDMs have reduced cytotoxicity compared to their homozygous G78 counterparts after HSV-1 infection for 18 hrs.

FIG. 3C: HSV-1 DNA was quantitated on DNA extracted from HSV-1-infected hMDMs after 6 and 18 hrs by qPCR. Results are % HSV-1 DNA normalized to GAPDH considering G78 donor as 100% for each donor pair. Homozygous R78 hMDMs have lower amounts of HSV-1 DNA at 6 hrs for all MOI tested and at 18 hrs for lower MOI (0.01 and 0.1), compared to homozygous G78 hMDMs.

FIG. 3D: Viral titers in the culture supernatant of HSV-1-infected hMDMs were determined by plaque assay on Vero cells. Results are number of plaque forming units (PFU) per ml of supernatant collected from HSV-1-infected hMDMs from three donor pairs (G78, solid lines; R78, dashed lines) after 6, 18 and 36 hrs of infection. Supernatants from homozygous R78 hMDMs contained less PFU for all MOI at 6 hrs and 18 hrs compared to supernatant from homozygous G78 counterparts. By 36 hrs, R78 supernatants contained fewer PFU than G78 supernatants only at lower MOI (0.01 and 0.1).

FIG. 3E: Viral titers in the culture supernatant of HSV-1-infected hMDMs were determined by plaque assay on Vero cells. Results are number of PFU per ml of supernatant collected from HSV-1-infected hMDMs after 18 hrs of infection from five pairs of genotyped donors (data from two individual experiments).

FIG. 4A-C show sequences of PILRA ligands and experiments which revealed C4A, and by inference C4B, as a new PILRA ligand. Statistical analysis is two-tailed unpaired t-test (p values <0.05=*, <0.005=**, <0.0005=***, <0.0001=****) performed on 3-4 independent experiments.

FIG. 4A: Shows a comparison of the peptide sequence around the O-glycosylated Thr (position 0) of known and putative (§) PILRA ligands.

FIG. 4B: 293T cells were transfected with putative ligands of PILRA (SORCS1 extracellular domain (ECD), APLP1 ECD or full length C4A) fused with C-terminal glycoprotein D (gD) tag and GPI anchor, or full length NPDC1 as positive control. 48 hrs post transfection, cells were harvested and incubated with soluble mIgG2a-tagged G78 variant of PILRA for receptor-ligand interactions. Cells were than stained with anti-mIgG2a (FITC). Binding of the G78 variant of PILRA to ligand-transfected cells was analyzed by flow cytometry. Results are fold-increase in binding to each putative ligand compared to vector control for each experiment.

FIG. 4C: 293T cells were transfected with full length C4A fused with C-terminal gD tag and GPI anchor. 48 hrs post transfection, cells were harvested and incubated with soluble mIgG2a-tagged variants of PILRA for receptor-ligand interactions. Cells were then stained with anti-mIgG2a (FITC). Binding of different PILRA variants to C4A-transfected cells was analyzed by flow cytometry. Results are the percentage of MFI of PILRA-mFc binding on ligand-transfected cells considering the G78 variant of PILRA binding as 100% for each experiment.

FIG. 5A-B show ligand binding blocking activity of anti-PILRA antibodies in the PILRA ECD-based competitive ELISA. Serially diluted antibodies were premixed with a fix concentration of the ligand-Fc and added to the ELISA plates with biotinylated PILRA ECD bound to the Neutravidin coated on the plate. Signals from the bound ligand-Fc are shown.

FIG. 5A: Shows the results of blocking mouse CD99 binding to mPILRA.

FIG. 5B: Shows the results of blocking mouse C12orf53 binding to mPILRA.

FIG. 6A-B show ligand binding blocking activity of anti-PILRA antibodies in the 293-PILRA cell-based competitive ELISA. Serially diluted antibodies were premixed with a fix concentration of the ligand-Fc and added to 293-PILRA stable cells. Signals from the bound ligand-Fc are shown.

FIG. 6A: Shows the results of blocking mouse CD99 binding to mPILRA.

FIG. 6B: Shows the results of blocking mouse C12orf53 binding to mPILRA.

FIG. 7A-C show SPR sensorgrams for PILRA binding to immobilized antibodies followed by antibody/ligand binding to the complex.

FIG. 7A: Shows the binding results when antibody 12C6.9 is directly immobilized.

FIG. 7B: Shows the binding results when blocking mAb1 is directly immobilized.

FIG. 7C: Shows the binding results when non-blocking mAb2 is directly immobilized.

FIG. 8A-B show antibody and ligand relationships based on SPR data.

FIG. 8A: Shows a network plot of the antibody/ligand relationship.

FIG. 8B: Shows a heatmap of direct antibody/ligand interactions.

### DETAILED DESCRIPTION

Provided herein are methods of treating a disease associated with myeloid cell dysfunction. In some embodiments, provided herein are methods of treating AD and HSV-1 infection. In particular, provided herein are methods of treating AD and HSV-1 infection by administering an effective amount of an agent to a subject wherein the agent specifically binds to one or more variants of PILRA thereby inhibiting the interaction between PILRA and its ligand. Also provided herein are methods of predicting a response of a subject or selecting a subject with AD and HSV-1 infection for treatment with an agent specifically binding to one or more variants of PILRA thereby inhibiting the interaction between PILRA and its ligand based upon detecting the presence or absence of one or more variants of PILRA. In some embodiments, provided herein are methods of treating AD and HSV-1 infection using agent specifically binding to one or more variants of PILRA thereby inhibiting the interaction between PILRA and its ligand. In particular, provided herein are methods of treating AD and HSV-1 infection using agent specifically binding to one or more variants of PILRA thereby inhibiting the interaction between PILRA and its ligand, wherein the agent is an antibody.

### I. Definitions

All numbering for amino acids of proteins and polypeptides mentioned herein relate to the full-length unprocessed protein, e.g. including the signal peptide, unless stated otherwise.

As used herein, “PILR” refers to paired immunoglobulin-like receptors (PILR) alpha (PILRA) and/or beta (PILRB). They are related type I transmembrane receptors bearing a highly similar extracellular domain (83% identity) but divergent intracellular signaling domains. When only one of the members is being referenced it will be designated as either PILRA or PILRB.

The terms “PILRA”, “Paired Immunoglobulin-like Type 2 Receptor Alpha”, “PILRA polypeptide” and “PILRA protein” as used herein, refers to any native PILRA from any vertebrate source, including mammals such as primates (e.g., humans) and rodents (e.g., mice and rats), unless otherwise indicated. The term encompasses “full-length, unprocessed PILRA” as well as any form of PILRA that results from processing in the cell. The term also encompasses naturally occurring variants of PILRA, e.g., allelic variants or splice variants. In some embodiments, the amino acid sequence of an exemplary human PILRA is SEQ ID NO:01 (G78 variant). In some embodiments, the amino acid sequence of an exemplary human PILRA relates to amino acid residues 20-303 (minus signal peptide) of SEQ ID NO:01. In some embodiments, the amino acid sequence of an exemplary human PILRA is selected from the group consisting of SEQ ID NO:01-SEQ ID NO:03. In some embodiments, the amino acid sequence of an exemplary human PILRA relates to amino acid residues 20-303 (minus signal peptide) of any one of SEQ ID NO:01-SEQ ID NO:03. The G78 variant of PILRA is considered herein the variant that accounts for an increased AD-risk. Therefore, the G78 variant of PILRA is also named herein as G78 variant of PILRA (AD-risk). The R78 variant of PILRA is considered herein the variant that accounts for the observed protection from AD-risk. Therefore, the R78 variant of PILRA is also named herein as R78 variant of PILRA (AD-protective).

The term “variant” or “variants” of a protein shall include all its allelic variants or splice variants. In some embodiments, the term “variant of PILRA” or “variants of PILRA” shall include all variants, e.g. all natural variants. In some embodiments, the term “variant of PILRA” or “variants of PILRA” shall include the G78 variant of PILRA having the sequence of SEQ ID NO:01, herein also referred to as G78 or G78 variant. In some embodiments, the term “variant of PILRA” or “variants of PILRA” shall include the R78 variant of PILRA having the sequence of SEQ ID NO:02, herein also referred to as R78 or R78 variant. In some embodiments, the term “variant of PILRA” or “variants of PILRA” shall include the L279 variant of PILRA having the sequence of SEQ ID NO:03, herein also referred to as L279 or L279 variant.

The term “PILRA variant” or “variant of PILRA” as used herein, refers to any of the PILRA variants having the sequence of SEQ ID NO:01 -SEQ ID NO:03 as described above and to PILRA polypeptides comprising amino acid sequences having one or more amino acid sequence substitutions, deletions (such as internal deletions and/or PILRA polypeptide fragments), and/or insertions (such as internal additions and/or PILRA fusion polypeptides) as compared to the sequence of the G78 variant of PILRA as defined herein. Such amino acid sequence substitutions, deletions, and/or insertions may be naturally occurring (e.g., PILRA allelic variants, PILRA orthologs and PILRA splice variants) or may be artificially constructed. Such PILRA variants having such amino acid sequence substitutions, deletions, and/or insertions may be prepared from the corresponding nucleic acid molecules having a DNA sequence that varies accordingly from the DNA sequence as defined below for the PILRA gene. In some embodiments, the PILRA variants, having such amino acid sequence substitutions, deletions, and/or insertions, have from 1 to 3, or from 1 to 5, or from 1 to 10, or from 1 to 15, or from 1 to 20, or from 1 to 25, or from 1 to 50, or from 1 to 75, or from 1 to 100, or more than 100 amino acid substitutions, deletions, and/or insertions, wherein the substitutions may be conservative, or non-conservative, or any combination thereof. Such variants include, for instance, polypeptides wherein one or more amino acid (naturally occurring amino acid and/or a non-naturally occurring amino acid) residues are inserted, or deleted, at the N- and/or C-terminus of the polypeptide. Ordinarily, such variant will have at least about 80% amino acid sequence identity, or at least about 90% amino acid sequence identity, or at least about 95% or more amino acid sequence identity with the wt polypeptide. Variants also include polypeptide fragments (e.g., subsequences, truncations, etc.), typically biologically active, of the corresponding wt. “PILRA variant” or “variant of PILRA” means a PILRA polypeptide as defined herein having at least about 80% amino acid sequence identity to a G78 variant of PILRA (SEQ ID NO:01). Ordinarily, a PILRA variant will have at least about 80% amino acid sequence identity, or at least about 85% amino acid sequence identity, or at least about 90% amino acid sequence identity, or at least about 95% amino acid sequence identity, or at least about 98% amino acid sequence identity, or at least about 99% amino acid sequence identity with the G78 variant of PILRA. In some embodiments, the PILRA variant comprises the amino acid G at position 78 (SEQ ID NO:01). In some embodiments, the PILRA variant comprises the amino acid R at position 78 (SEQ ID NO:02). In some embodiments, the PILRA variant comprises the amino acid S at position 279 (SEQ ID NO:01). In some embodiments, the PILRA variant comprises the amino acid L at position 279 (SEQ ID NO:03).

In some embodiments, the amino acid sequence of the human PILRA comprises the amino acid G at position 78. In some embodiments, the amino acid sequence of the human PILRA comprises the amino acid R at position 78. In some embodiments, the amino acid sequence of the human PILRA comprises the amino acid S at position 279. In some embodiments, the amino acid sequence of the human PILRA comprises the amino acid L at position 279.

In some embodiments, the nucleic acid sequence of the human PILRA comprises a sequence encoding the amino acid G at position 78. In some embodiments, the nucleic acid sequence of the human PILRA comprises a sequence encoding the amino acid R at position 78. In some embodiments, the nucleic acid sequence of the human PILRA comprises a sequence encoding the amino acid S at position 279. In some embodiments, the nucleic acid sequence of the human PILRA comprises a sequence encoding the amino acid L at position 279.

“Percent (%) amino acid sequence identity” herein is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained as described below by using the sequence comparison computer program ALIGN-2. The ALIGN-2 sequence comparison computer program was authored by Genentech, Inc. has been filed with user documentation in the U.S. Copyright Office, Washington D.C., 20559, where it is registered under U.S. Copyright Registration No. TXU510087, and is publicly available through Genentech, Inc., South San Francisco, Calif The ALIGN-2 program should be compiled for use on a UNIX operating system, e.g., digital UNIX V4.0D. All sequence comparison parameters are set by the ALIGN-2 program and do not vary.

The term “polypeptide” as used herein, refers to any native polypeptide of interest (e.g., PILRA) from any vertebrate source, including mammals such as primates (e.g., humans) and rodents (e.g., mice and rats), unless otherwise indicated. The term encompasses “full-length,” unprocessed polypeptide as well as any form of the polypeptide that results from processing in the cell. The term also encompasses naturally occurring variants of the polypeptide, e.g., splice variants or allelic variants.

The term “APLP1” as used herein refers to the protein having the amino acid sequence of SEQ ID NO:04, which includes a potential signal sequence. Amyloid beta precursor like protein 1 (APLP1) is an alpha 2A adrenergic receptor binding protein that regulates proteolysis of amyloid precursor proteins, negatively regulates endocytosis; map position of corresponding gene correlates with AD. APLP1 is known to be O-glycosylated at Thr-215 (see e.g., Nilsson et al., Nat. Methods 6:809-811 (2009)) within a PILRA interaction motif. In some embodiments, the amino acid sequence of human APLP1 is UNIPROT P51693.

The term “C16orf54” as used herein refers to the protein having the amino acid sequence of SEQ ID NO:05, which includes a potential signal sequence. Chromosome 16 open reading frame 54 (C16orf54) is a single pass, type II transmembrane protein of unknown function, with O-glycosylation at Thr-4 identified by mass spectrometry (see e.g., Halim et al., Mol. Cell. Proteomics 11:1-17 (2012)) within a PILRA interaction motif. In some embodiments, the amino acid sequence of human C16orf54 is UNIPROT Q6UWD8.

The term “C4A” as used herein refers to the protein having the amino acid sequence of SEQ ID NO:06, which includes a potential signal sequence. The term “C4B” as used herein refers to the protein having the amino acid sequence of SEQ ID NO:07, which includes a potential signal sequence. The Complement component 4 (C4) protein is encoded by 2 genes in humans, C4A and C4B. The putative PILRA-binding motif is identical for C4A and C4B. C4 genes are located in the HLA class III region. C4 components are thought to play a role in AD (see e.g., Zorzetto et al., Curr. Alzheimer Res. 14(3):303-308 (2017)). Complement factor 4A (C4A) has important roles in coronary arteriosclerosis (see e.g., Stakhneva et al., Bull. Exp. Biol. Med. 162(3):343-345 (2017), hereditary angioedema (see e.g., Aabom et al., Clin. Biochem. 50(15): 816-821 (2017)), and schizophrenia (see e.g., Sekar et al., Nature 530(7589):177-183 (2016)). O-glycosylation at T1244 of C4A has been identified by mass spectrometry in samples of human cerebrospinal fluid (see e.g., Halim et al., J. Proteome Res. 12:573-584 (2013)) and is located within a PILRA interaction motif. In some embodiments, the amino acid sequence of human C4A is UNIPROT P0C0L4. In some embodiments, the amino acid sequence of human C4B is UNIPROT P0C0L5.

The term “CD99” or “mCD99” or “mouse CD99” or “murine CD99” as used herein refers to the protein having the amino acid sequence of SEQ ID NO:58. CD99 is a single chain glycoprotein that participates in the migration of leukocytes through endothelial junctions by hemophilic interaction (see e.g., Schenkel, et al., Nat. Immunol., 3, 143-150 (2002)). In some embodiments, the amino acid sequence of mCD99 is UNIPROT Q8BIF0. In some embodiments, the amino acid sequence of CD99 is the human sequence as shown in SEQ ID NO:59 and UNIPROT Q8TCZ2.

The term “CLEC4G” as used herein refers to the protein having the amino acid sequence of SEQ ID NO:08, which includes a potential signal sequence. C type lectin superfamily 4 member G (CLEC4G) is a homodimerizing protein that functions as a pathogen associated molecular pattern receptor, may play a role in cell-cell adhesion, antigen processing, and presentation (see, e.g., Liu et al., J. Biol. Chem. 279(18) 18748-58 (2004)). In some embodiments, the amino acid sequence of human CLEC4G is UNIPROT Q6UXB4.

The term “COLEC12” as used herein refers to the protein having the amino acid sequence of SEQ ID NO:09. Collectin subfamily member 12 (COLEC12) is a type II transmembrane glycoprotein that binds bacteria through its lectin domain and may play a role in host defense. As a scavenger receptor, COLEC12 may bind amyloid-β and promote phagocytosis (see e.g., Nakamura et al., J. Neurosci. Res. 84(4):874-890 (2006)), and microglial expression of COLEC12 is induced in mouse neurodegenerative models. Treatment of COLEC12 with sialidase A abolishes its interaction with PILRA (see e.g., Sun et al., J. Biol. Chem. 287(19):15837-15850 (2012)). In some embodiments, the amino acid sequence of human COLEC12 is UNIPROT Q5KU26.

The term “DAG1” as used herein refers to the protein having the amino acid sequence of SEQ ID NO:10, which includes a potential signal sequence. Dystroglycan 1 or dystrophin-associated glycoprotein 1 (DAG1) is an extracellular matrix glycoprotein that acts in muscle contraction, may be involved in synaptic transmission and establishment of cell polarity, aberrant protein expression correlates with muscular dystrophies and several neoplasms. O-glycosylation at T455 of DAG1 has been identified by mass spectrometry (see e.g., Nilsson et al., Glycobiology 20:1160-1169 (2010)) and is located within a PILRA interaction motif. In some embodiments, the amino acid sequence of human DAG1 is UNIPROT Q14118.

The term “EVA1C” as used herein refers to the protein having the amino acid sequence of SEQ ID NO:11, which includes a potential signal sequence. Protein eva-1 homologue C (EVA1C) is a single pass, type I transmembrane protein with an extracellular carbohydrate-binding domain that may have a role in axon guidance during nervous system development (see e.g., James et al., PLoS One 8(9):e74115 (2013)). In some embodiments, the amino acid sequence of human EVA1C is UNIPROT P58658.

The term “FceRII” as used herein refers to the protein having the amino acid sequence of SEQ ID NO:12. Fc fragment of IgE low affinity II receptor (FceRII, or FCER2), acts in thymocyte maturation, histamine secretion, and TNF production, regulates NO production in monocytes, upregulated in hypogammaglobulinaemia, Kawasaki disease, Graves thyrotoxicosis, and chronic uremia. In some embodiments, the amino acid sequence of human FceRII is

The term “HSV-1 gB as used herein refers to the human herpes simplex virus type-1 glycoprotein B, having the amino acid sequence of SEQ ID NO:13. HSV-1 gB is essential for initial attachment of a virus to the host cell surface proteoglycans and is involved in fusion of viral and cellular membranes leading to virus entry into host cell. In some embodiments, the amino acid sequence of HSV-1 gB is UNIPROT P06437.

The term “IL17RA” as used herein refers to the protein having the amino acid sequence of SEQ ID NO:14, which includes a potential signal sequence. Interleukin-17 receptor A (IL17RA) is a single pass, type I transmembrane protein that forms a heterodimeric complex with IL17RC to act as a receptor for homodimeric IL-17A, homodimeric IL-17F, or heterodimeric IL-17A/F cytokines. IL17RA may also complex with IL17RE to form a receptor for homodimeric IL-17C. IL17RA activation leads to expression of inflammatory cytokines and chemokines. In some embodiments, the amino acid sequence of human IL17RA is UNIPROT Q96F46.

The term “LILRB5” as used herein refers to the protein having the amino acid sequence of SEQ ID NO:15, which includes a potential signal sequence. Leukocyte immunoglobulin-like receptor subfamily B member 5 (LILRB5) is a single pass, type I transmembrane protein with four Ig-like C2-type domains in its extracellular portion, which binds to class I MHC proteins (see e.g., Zhang et al., PLoS One 10(6):e0129063 (2015)). The cytoplasmic portion of LILRB5 transduces inhibitory signals through its ITIM domain. Variants and expression levels of LILRB5 are associated with statin intolerance and myalgia, serum levels of creatine kinase and lactate dehydrogenase, and mycobacteria exposure. In some embodiments, the amino acid sequence of human LILRB5 is UNIPROTO 75023.

The term “LRRC15” as used herein refers to the protein having the amino acid sequence of SEQ ID NO:16, which includes a potential signal sequence. Leucine-rich repeat-containing protein 15 (LRRC15) is a single pass, type I transmembrane protein whose expression in astrocytes is induced by treatment with amyloid-β or pro-inflammatory cytokines and whose extracellular portion consists of fifteen leucine-rich repeat domains involved in cell-cell or extracellular matrix interactions (see e.g., Satoh et al., Biochem. Biophys. Res. Commun. 290(2):756-62 (2002)). In some embodiments, the amino acid sequence of human LRRC15 is UNIPROT Q8TF66.

The term “LRRTM4” as used herein refers to the protein having the amino acid sequence of SEQ ID NO:17, which includes a potential signal sequence. Leucine rich repeat transmembrane neuronal 4 may stimulate beta-secretase mediated processing of beta-amyloid-precursor protein, may play a role in brain development and is associated with AD. LRRTM4 contains nine leucine rich repeats. In some embodiments, the amino acid sequence of human LRRTM4 is UNIPROT Q86VH4.

The term “NPDC1” as used herein refers to the protein having the amino acid sequence of SEQ ID NO:18, which includes a potential signal sequence. NPDC1 is specifically expressed in neural cells when they stop to divide and begin to differentiate. It may also regulate transcription, cell proliferation, neuron differentiation, and organ morphogenesis. Its expression is developmentally regulated and persists in the adult; it increases in the embryonic brain, in distinct, defined regions, and is correlated with growth arrest and terminal differentiation. NPDC1 has long hydrophobic stretch of amino acids (residues 13-29), a coiled-coil region (amino acids 93-120), a transmembrane domain (amino acids 191-207), an acidic domain (amino acids 277-307), and MAP-kinases consensus sites (amino acids 234-244) (see, e.g., Evrard and Rouget, J. Neuro. Res. 79:747-755 (2005)). It may be clipped and exist in a soluble form. Treatment of NPDC1 with sialidase A abolishes its interaction with PILRA (see e.g., Sun et al., J. Biol. Chem. 287(19):15837-15850 (2012)). In some embodiments, the amino acid sequence of human NPDC1 is UNIPROT Q9NQX5.

The term “PIANP” as used herein refers to the protein having the amino acid sequence of SEQ ID NO:19, which includes a potential signal sequence. PILRA-associated neural protein (PIANP) is a single pass, type I transmembrane protein with O-linked glycosylation on T140 that mediates its association with PILRA (see e.g., Kogure et al., Biochem. Biophys. Res. Commun. 405:428-33 (2011)). Though typically present in neurons, PIANP expression is also induced in microglia in several neurodegenerative disease models. In some embodiments, the amino acid sequence of human PIANP is UNIPROT Q8IYJ0. PIANP is herein also referred to as C12orf53, or human chromosome 12 open reading frame 53. The term “mPIANP” or “mC12orf53” as used herein refers to the murine orthologue of human PIANP not to open reading frame 53 of mouse chromosome 12. Synonyms used for PIANP are e.g. PANP, LEDA-1 and C530028O21Rik (in mouse).

The term “PRSS55” as used herein refers to the protein having the amino acid sequence of SEQ ID NO:20, which includes a potential signal sequence. Serine protease 55 (PRSS55) is a single pass, type I transmembrane protein with endopeptidase activity in its extracellular domain. In some embodiments, the amino acid sequence of human PRSS55 is UNIPROT Q6UWB4.

Measuring the binding of a ligand (as defined herein above) to PILRA may be performed using (without limitation) such suitable assays as quantitative comparisons comparing kinetic and equilibrium binding constants. The kinetic association rate (kₒₙ) and dissociation rate (k_{off}), and the equilibrium binding constants (K_{d}) may be determined using surface plasmon resonance on a BlAcore™ instrument following the standard procedure in the literature. Binding properties of these interactions may also be assessed by flow cytometry and/or by solid phase binding assay.

An “agent”, a “binding agent”, an “anti-PILRA binding agent”, an “agent specifically binding to PILRA”, or an “agent specifically binding to one or more variants of PILRA is an agent that binds to PILRA in such a way that it interferes with the ligand binding of PILRA, e.g., the agent partially or fully blocks or inhibits the binding of PILRA to its ligands. For example, the agent may refer to any molecule that partially or fully blocks or inhibits the binding of PILRA to its ligands. Examples of such agents include antibodies (e.g., anti-PILRA antibodies), polypeptides (e.g., PILRA binding polypeptides), polynucleotides (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecules (e.g., small molecules binding to PILRA). In some embodiments, the anti-PILRA binding agent is an antibody or small molecule which binds to PILRA.

Anti-PILRA binding agent (e.g., anti-PILRA antibodies) may be experimentally tested and validated using in vivo and in vitro assays. Suitable assays include, but are not limited to, activity assays and binding assays. In some embodiments, assays can be used as known in the art (e.g. see Shiratori et al., J Exp Med, 16, 199(4):525-533 (2004), and Wang et al., Nat Immunol, 14(1):34-40 (2013)).

As used herein, the term “block” or “inhibit” refers to a decrease in one or more given measurable activity by at least 10% relative to a reference and/or control. Where inhibition is desired, such inhibition is preferably at least 20%, 30%, 40%, 50%, 60%, 70%, 80%, 90% or more, up to and including 100%, i.e., complete inhibition or absence of the given activity. As used herein, the term “substantially inhibits/blocks” refers to a decrease in a given measurable activity by at least 50% relative to a reference. For example, “substantially inhibits” refers to a decrease in a given measurable activity of at least 50%, 55%, 60%, 65%, 70%, 75%, 80%, 85%, 90%, 95% and up to and including 100% relative to a reference. As used herein, “blocks/prevents/inhibits/impairs/lowers the interaction”, with reference to the binding of a ligand that binds to a receptor refers to a decrease in binding by at least 10% relative to a reference. An agent may block the binding of a ligand to a receptor-expressing cells. “Inhibits the interaction” and/or “block the binding” preferably refers to a decrease in binding of at least 20%, 30%, 40%, 50%, 60%, 70%, 80%, 90% or more, up to and including 100%. A “receptor” as provided for herein means PILRA. A “ligand” as provided for herein is selected from the group consisting of APLP1, C16orf54, C4A, C4B, CLEC4G, COLEC12, DAG1, EVA1C, FceRII, IL17RA, LILRBS, LRRC15, LRRTM4, NPDC1, PIANP, PRSS55 and HSV-1 gB. A general feature of a ligand is glycan modification, e.g., sialydated glycans.

A “ligand” as provided for herein is selected from the group consisting of APLP1, C16orf54, C4A, C4B, CD99, CLEC4G, COLEC12, DAG1, EVA1C, FceRII, IL17RA, LILRB5, LRRC15, LRRTM4, NPDC1, PIANP, PRSS55 and HSV-1 gB. A general feature of a ligand is glycan modification, e.g., sialydated glycans.

“Affinity” or “Binding Affinity” refers to the strength of the sum total of non-covalent interactions between a single binding site of a molecule (e.g., antibody, polypeptide, polynucleotide, and small molecule) and its binding partner (e.g., an antigen). Unless indicated otherwise, as used herein, “binding affinity” refers to intrinsic binding affinity which reflects a 1:1 interaction between members of a binding pair (e.g., either of antibody, polypeptide, polynucleotide, small molecule and the antigen). The affinity of a molecule X for its partner Y can generally be represented by the dissociation constant (Kd). Affinity can be measured by common methods known in the art, including those described herein (e.g., peptide substrate assay, direct assay or coupled assay).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” or an “antibody that binds to the same binding region” as a reference antibody refers to an antibody that blocks binding of the reference antibody to its binding partner (e.g., an antigen) in a competition assay by 50% or more, and conversely, the reference antibody blocks binding of the antibody to its binding partner in a competition assay by 50% or more.

The terms “anti-PILRA antibody” and “an antibody that binds to PILRA” refer to an antibody that is capable of binding PILRA with sufficient affinity such that the antibody is useful as a diagnostic and/or therapeutic agent in targeting PILRA. In some embodiments, the extent of binding of an anti-PILRA antibody to an unrelated polypeptide (polypeptide other than PILRA) is less than about 10% of the binding of the antibody to PILRA as measured, e.g., by a radioimmunoassay (RIA). In some embodiments, an antibody that binds to PILRA has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In some embodiments, an anti-PILRA antibody binds to a binding region (e.g. an epitope) of PILRA that is conserved among different species of PILR polypeptides.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

A “binding region” is the portion of the binding partner (e.g., an antigen) to which an agent specifically binding to PILRA (e.g. an antibody, polypeptide, polynucleotide, or small molecule) selectively binds. For a polypeptide binding partner, a linear binding region can be a peptide portion of about 4-15 (e.g., 4, 5, 6, 7, 8, 9, 10, 11, 12, 13, 14 or 15) amino acid residues. A non-linear, conformational binding region may comprise residues of a polypeptide sequence brought to close vicinity in the three-dimensional (3D) structure of the polypeptide binding partner. In some embodiments, the binding region is the SA binding region within PILRA.

The terms “full length antibody,” “intact antibody,” and “whole antibody” are used herein interchangeably to refer to an antibody (e.g., an anti-PILRA antibody) having a structure substantially similar to a native antibody structure or having heavy chains that contain an Fc region.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In some embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3). Unless otherwise indicated, HVR residues and other residues in the variable domain (e.g., FR residues) are numbered herein according to Kabat et al., supra.

The term “isolated” as used in reference to antibody, polypeptide, polynucleotide or small molecule is one which has been separated from a component of its natural environment. In some embodiments, an antibody, polypeptide, polynucleotide or small molecule is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC).

The term “monoclonal antibody” as used herein refers to an antibody obtained from a population of substantially homogeneous antibodies, i.e., the individual antibodies comprising the population are identical and/or bind the same binding region (e.g., epitope), except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies described herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies.

The term “variable region” or “variable domain” refers to the domain of an antibody heavy or light chain that is involved in binding the antibody to an antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “small molecule” refers to any molecule with a molecular weight of about 2000 daltons or less, preferably of about 500 daltons or less.

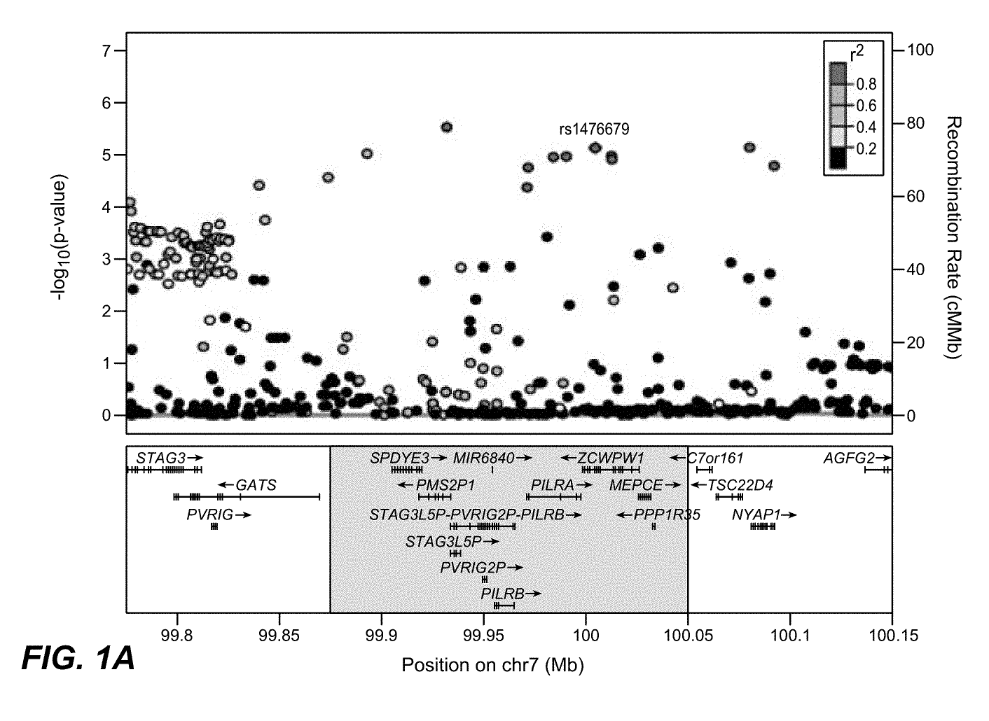

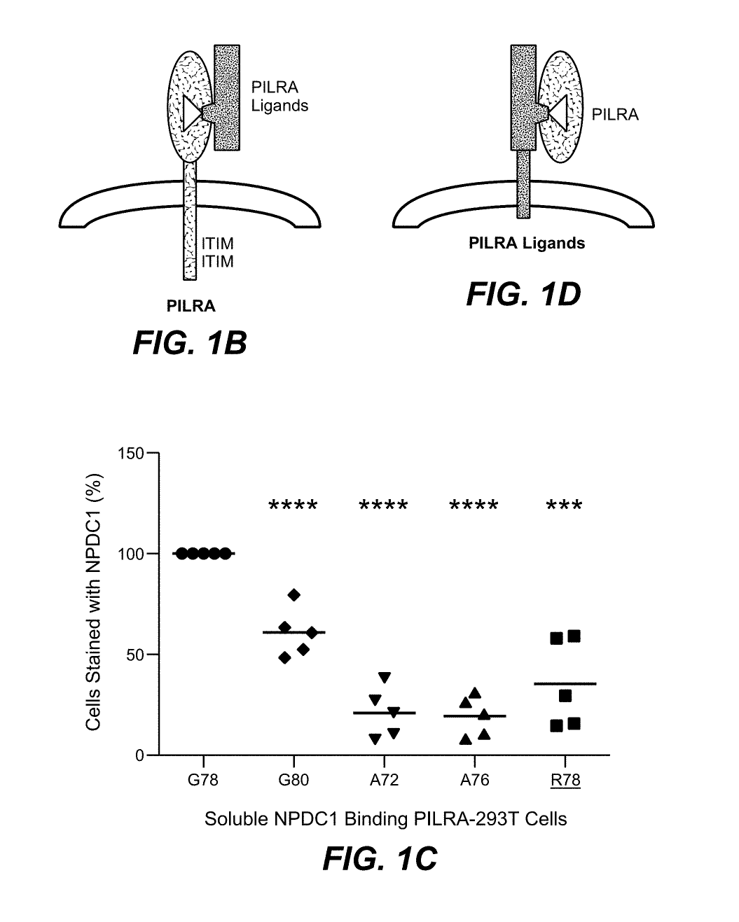

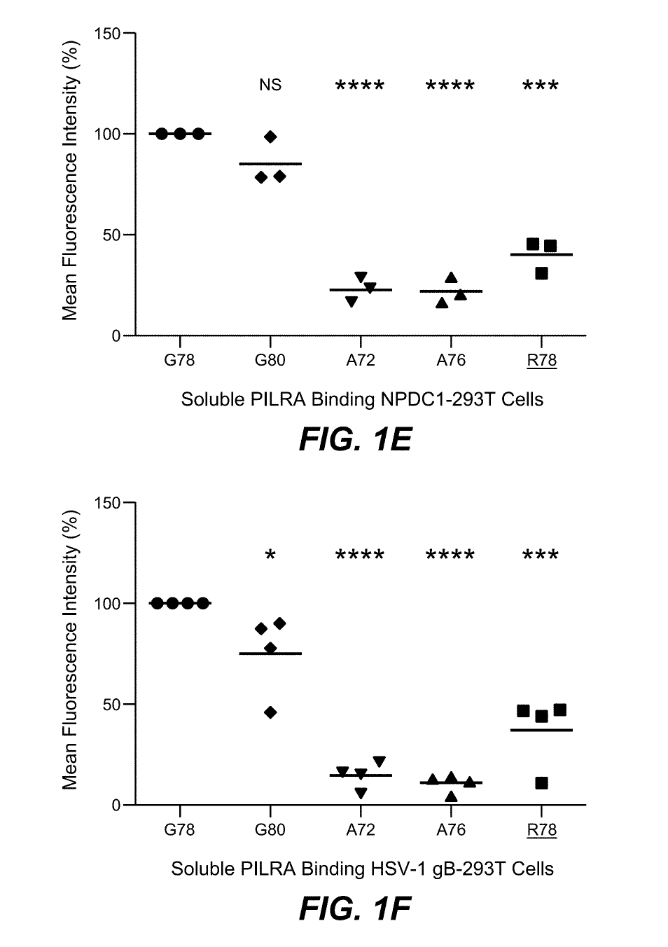

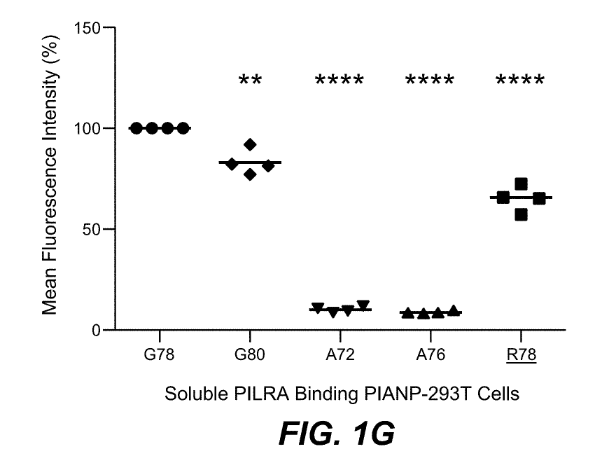

The terms “PILR gene” or “PILR nucleic acid molecule” or “polynucleotide” refers to a nucleic acid molecule comprising or consisting of a nucleotide sequence encoding a specific PILR polypeptide. Exemplary nucleotide sequences are set forth e.g. in FIG. 1A of Fournier et al., J. Immunol. 165:1197-1209 (2000) and NM_013439 for human PILRA; multiple cDNAs have been identified for PILRB (see e.g., Wilson et al., Physiol. Genomics 27:201-18 (2006)) and annotated by NCBI, e.g., NM_178238.1, NM_178238.2, for human PILRB.

The term “PILRA genomic sequence” as used herein, refers to either the cDNA and/or the genomic form of the PILRA gene, which may include introns as well as upstream and downstream regulatory sequences.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polynucleotide analysis or protocol, one may use the results of the polynucleotide expression analysis or protocol to determine whether a specific therapeutic regimen should be performed.

The term “single nucleotide polymorphism” also referred to herein as “SNP” as used herein refers to a single base substitution within a DNA sequence that leads to genetic variability. A nucleotide position in a genome at which more than one sequence is possible in a population is referred to herein as a “polymorphic site” or “polymorphism”. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site that alters a single nucleotide is referred to herein as a SNP. When there are two, three or four alternative nucleotide sequences at a polymorphic site, each nucleotide sequence is referred to as a “polymorphic variant” or “nucleic acid variant”. Each possible variant in the DNA sequence is referred to as an “allele”. Where two polymorphic variants exist, the polymorphic variant represented in a majority of samples from a population is referred to as a “prevalent allele” or “major allele” and the polymorphic variant that is less prevalent in the population is referred to as an “uncommon allele” or “minor allele”. An individual who carries two prevalent alleles or two uncommon alleles is “homozygous” with respect to the polymorphism. An individual who carries one prevalent allele and one uncommon allele is “heterozygous” with respect to the polymorphism. With C/G or A/T SNPs, the alleles are ambiguous and dependent on the strand used to extract the data from the genotyping platform. With these C/G or A/T SNPs, the C or G nucleotide or the A or T nucleotide, respectively, may be the risk allele and is determined by correlation of allele frequencies. The allele that correlates with an increased risk for a disease or is associated with an odds ratio or relative risk of >1 is referred to as the “risk allele” or “effect allele”. The “risk allele” or “effect allele” may be the minor allele or major allele. For example the risk allele is rs1476679 associated with age of onset and the risk allele of rs1476679 is associated with increased neuritic plaque and neurofibrillary tangles.

“Linkage disequilibrium or “LD” when used herein refers to alleles at different loci that are not associated at random, i.e. not associated in proportion to their frequencies. If the alleles are in positive linkage disequilibrium, then the alleles occur together more often than expected assuming statistical independence. Conversely, if the alleles are in negative linkage disequilibrium, then the alleles occur together less often than expected assuming statistical independence.

“Odds ratio” or “OR” when used herein refers to the ratio of the odds of the disease for individuals with the marker (allele or polymorphism) relative to the odds of the disease in individuals without the marker (allele or polymorphism).

“Increased risk” when used herein refers to when the presence in the genome of an individual of a particular base, at a particular location in the genome correlates with an increased probability of that individual developing a disease associated with myeloid cell dysfunction, e.g. AD or HSV-1 infection, vis-à-vis a population not having that base at that location in the genome, that individual is said to be at “increased risk” of developing a disease associated with myeloid cell dysfunction, i.e. to have an increased susceptibility. In the present case, such increased probability exists when the base is present in one or the other or both alleles of the individual. Furthermore, the probability is increased when the base is present in both alleles of the individual rather than one allele of the individual.

“Decreased risk” when used herein refers to when the presence in the genome of an individual of a particular base, at a particular location in the genome correlates with an decreased probability of that individual developing a disease associated with myeloid cell dysfunction, e.g. AD or HSV-1 infection, vis-à-vis a population not having that base at that location in the genome, that individual is said to be at “decreased risk” of developing a disease associated with myeloid cell dysfunction, i.e. to have a decreased susceptibility. Such an allele is sometimes referred to in the art as being “protective”. As with increased risk, it is also possible for a decreased risk to be characterized as dominant or recessive.

An “altered risk” means an increased or a decreased risk.

The term “genotyping” as used herein refers to methods of determining differences in the genetic make-up (“genotype”) of an individual, including but not limited to the detection of the presence of DNA insertions or deletions, polymorphisms (SNPs or otherwise), alleles (including minor or major or risk alleles in the form of SNPs, by examining the individual's DNA sequence using analytical or biological assays (or other methods for analysis of SNPs as described herein)). For instance, the individual's DNA sequence determined by sequencing or other methodologies (for example other methods for analysis of SNPs as described herein), may be compared to another individual's sequence or a reference sequence. Methods of genotyping are generally known in the art (for example other methods for analysis of SNPs as described herein), including but are not limited to restriction fragment length polymorphism identification (RFLP) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads. Similarly these techniques may be applied to analysis of transcripts that encode SNPs or other genetic factors. Samples can be conveniently assayed for a SNP using polymerase chain reaction (PCR) analysis, array hybridization or using DNA SNP chip microarrays, which are commercially available, including DNA microarray snapshots. A microarray can be utilized for determining whether a SNP is present or absent in a nucleic acid sample. A microarray may include oligonucleotides, and methods for making and using oligonucleotide microarrays suitable for diagnostic use are disclosed in U.S. Pat. Nos. 5,492,806; 5,525,464; 5,589, 330; 5,695,940; 5,849,483; 6,018,041; 6,045,996; 6,136,541; 6,152,681; 6,156, 501; 6,197,506; 6,223,127; 6,225,625; 6,229, 911; 6,239,273; WO 00/52625; WO 01/25485; and WO 01/29259.

A “phenotype” is a trait which can be compared between individuals, such as presence or absence of a condition, for example, occurrence of a disease associated with myeloid cell dysfunction, e.g. AD or HSV-1 infection.

The term “reference level”, as used herein refers to a predetermined value. In this context “level” encompasses the absolute amount, the relative amount or concentration as well as any value or parameter which correlates thereto or can be derived therefrom. As the skilled artisan will appreciate the reference level is predetermined and set to meet routine requirements in terms of e.g. specificity and/or sensitivity. These requirements can vary, e.g. from regulatory body to regulatory body. It may for example be that assay sensitivity or specificity, respectively, has to be set to certain limits, e.g. 80%, 90%, 95% or 98%, respectively. These requirements may also be defined in terms of positive or negative predictive values. Nonetheless, based on the teaching given in the present application it will always be possible for a skilled artisan to arrive at the reference level meeting those requirements. In some embodiments, the reference level is determined in reference samples from healthy individuals. In some embodiments, the reference level has been predetermined in reference samples from the disease entity to which the subject belongs. In some embodiments, the reference level can e.g. be set to any percentage between 25% and 75% of the overall distribution of the values in a disease entity investigated. In some embodiments, the reference level can e.g. be set to the median, tertiles or quartiles as determined from the overall distribution of the values in reference samples from a disease entity investigated. In some embodiments, the reference level is set to the median value as determined from the overall distribution of the values in a disease entity investigated. The reference level may vary depending on various physiological parameters such as age, gender or subpopulation. In some embodiment, the reference sample is from essentially the same type of cells, tissue, organ or body fluid source as the sample from the individual or patient subjected to the methods described herein. In some embodiments, the reference level is based on the interaction between the G78 variant of PILRA and any one of its ligands.

The term “sample” or “biological sample” as used herein, refers to a formulation that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, lacrimal secretion, semen, sweat, tumor lysates, and tissue culture medium, tissue biopsy, tissue extracts such as homogenized tissue, tumor tissue, cellular extracts, and combinations thereof.

By “tissue sample” or “cell sample” is meant a collection of similar cells obtained from a tissue of a subject. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some embodiments, the subject is a human.

The term “patient” as used herein, refers to an animal, such as a mammal. In some embodiments, patient refers to a human.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is non-toxic to a subject. A pharmaceutically acceptable carrier includes, but is not limited to, a buffer, excipient, stabilizer, or preservative.

“Treatment” (and variations such as “treat” or “treating”) refers to clinical intervention in an attempt to alter the natural course of the subject or cell being treated. Desirable effects of treatment include one or more of preventing occurrence or recurrence of disease, alleviation of symptoms, diminishment of any direct or indirect pathological consequences of the disease, stabilized (i.e., not worsening) state of disease, decreasing the rate of disease progression, amelioration or palliation of the disease state, prolonging survival as compared to expected survival if not receiving treatment and improved prognosis.

The term “administering” as used herein is used in the broadest sense and inter alia encompasses enteral, topical administration and “parenteral administration”. “Parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal injection, infusion, ocular, intraocular, intravitreal, juxtascleral, subtenon and superchoroidal. “IVT or ITV” when used herein refers to intravitreal.

The term “effective amount” is intended to mean an amount of an agent sufficient to substantially block the interaction between a ligand (as defined herein) and PILRA. An effective amount may also encompass either “therapeutically effective amount” and/or “prophylactically effective amount”. A “therapeutically effective amount” refers to an amount effective, at dosages and for periods of time necessary, to achieve the desired therapeutic result, such as a reduction in disease progression and/or alleviation of the symptoms associated with a disease. A therapeutically effective amount of anti-PILRA binding agents may vary according to factors such as the disease state, age, sex, and weight of the individual, and the ability of the agents to elicit a desired response in the individual. Dosage regimens may be adjusted to provide the optimum therapeutic response. A therapeutically effective amount is also one in which any toxic or detrimental effects of the agents are outweighed by the therapeutically beneficial effects. A “prophylactically effective amount” refers to an amount effective, at dosages and for periods of time necessary, to achieve the desired prophylactic result, such as preventing and/or inhibiting (reducing) the rate of disease onset or progression. A prophylactically effective amount may be determined as described above for the therapeutically effective amount. For any particular subject, specific dosage regimens may be adjusted over time according to the individual need and the professional judgment of the person administering of the compositions.

The phrase “selecting a patient”, “identifying a patient”, “selecting a subject”, or “identifying a subject” as used herein refers to using the information or data generated relating to the presence of a risk allele in a sample of a patient to identify or select the patient as more likely to benefit to benefit from a treatment comprising the agent, e.g. an anti-PILRA antibody. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes an indication that a risk allele is present or absent in the sample. In some embodiments, the information or data includes an indication that the patient is more likely to respond to a therapy comprising the agent, e.g. an anti-PILRA antibody.

The use of the terms “a” and “an” and “the” and similar terms in the context of describing embodiments herein are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. It is understood that aspects and embodiments provided herein include “consisting” and/or “consisting essentially of” aspects and embodiments.

As is understood by one skilled in the art, reference to “about” a value or parameter herein includes (and describes) embodiments that are directed to that value or parameter per se. For example, description referring to “about X” includes description of “X”.

The phrase “substantially different,” refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values may be, for example, greater than about 10%, greater than about 20%, greater than about 30%, greater than about 40%, and/or greater than about 50% as a function of the value for the reference/comparator molecule.

The phrase “substantially similar,” as used herein, refers to a sufficiently high degree of similarity between two numeric values (generally one associated with a molecule and the other associated with a reference/comparator molecule) such that one of skill in the art would consider the difference between the two values to not be of statistical significance within the context of the biological characteristic measured by said values (e.g., Kd values). The difference between said two values may be, for example, less than about 20%, less than about 10%, and/or less than about 5% as a function of the reference/comparator value. The phrase “substantially normal” refers to substantially similar to a reference (e.g., normal reference).

### II. Methods of Using Anti-PILRA Binding Agents

Provided herein are methods of using an agent (e.g. an anti-PILRA antibody) for inhibiting the interaction between one or more variants of PILRA and any one of its ligands. For example, provided herein are methods for treating a disease associated with myeloid cell dysfunction in a subject comprising administering an effective amount of an agent to the subject. In some embodiments, the agent inhibits the interaction between one or more variants of PILRA and any one of its ligands. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody (e.g., a monoclonal antibody).

Further, provided herein are methods of selecting a subject having a disease associated with myeloid cell dysfunction for a treatment with an agent inhibiting the interaction between one or more variants of PILRA and any one of its ligands, comprising determining the presence or absence of the one or more variants of PILRA in a biological sample from the subject, wherein the presence of the one or more variants of PILRA indicates that the subject is suitable for treatment with the agent. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody (e.g., a monoclonal antibody).

Further provided herein are methods of predicting the response of a subject having a disease associated with myeloid cell dysfunction to a treatment with an agent specifically binding to one or more variants of PILRA, the method comprising (a) obtaining a biological sample from the subject, (b) optionally identifying the one or more variants of PILRA in the biological sample, (c) measuring whether the agent specifically binding to the one or more variants of PILRA inhibits the interaction between PILRA and any one of its ligands as compared to a reference level, and (d) predicting that the subject will respond to the treatment when the interaction between PILRA and any one of its ligands is inhibited as compared to the reference level and predicting that the subject will not respond to the treatment when the interaction between PILRA and any one of its ligands is not inhibited as compared to the reference level. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody (e.g., a monoclonal antibody).

Further provided herein are methods of predicting the response of a subject having a disease associated with myeloid cell dysfunction to a treatment with an agent specifically binding to one or more variants of PILRA, the method comprising (a) measuring whether the agent specifically binding to the one or more variants of PILRA inhibits the interaction between PILRA and any one of its ligands as compared to a reference level, and (b) predicting that the subject will respond to the treatment when the interaction between PILRA and any one of its ligands is inhibited as compared to the reference level and predicting that the subject will not respond to the treatment when the interaction between PILRA and any one of its ligands is not inhibited as compared to the reference level. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody (e.g., a monoclonal antibody).

Further provided herein are methods for detecting the presence or absence of one or more variants of PILRA indicating that a subject having a disease associated with myeloid cell dysfunction is suitable for treatment with an agent inhibiting the interaction between PILRA and any one of its ligands, comprising (a) contacting a sample from the subject with a reagent capable of detecting the presence or absence of the one more variants of PILRA; and (b) determining the presence or absence of the one or more variants of PILRA, wherein the presence of the one or more variants of PILRA indicates that the subject is suitable for treatment with an agent inhibiting the interaction between PILRA and any one of its ligands. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the anti-PILRA binding agent is an antibody (e.g., a monoclonal antibody). In some embodiments, the reagent is selected from an oligonucleotide, a DNA probe, an RNA probe, and a ribozyme. In some embodiments, the reagent is labeled. In some embodiments, the reagent is a TaqMan Probe.

Further provided herein are methods for selecting an agent for treating a disease associated with myeloid cell dysfunction, comprising determining whether the agent inhibits the interaction between PILRA and any one of its ligands, wherein the agent that inhibits the interaction between PILRA and any one of its ligands is suitable for treating the disease associated with myeloid cell dysfunction. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody (e.g., a monoclonal antibody).

In some embodiments of any of the methods, the disease associated with myeloid cell dysfunction is selected from the group consisting of AD and HSV-1 infection. In some embodiments, the myeloid cell dysfunction is associated with a decreased myeloid cell activity.

In some embodiments of any of the methods, the one or more variants of PILRA are encoded by a polynucleotide sequence comprising one or more SNPs. In some embodiments, the one or more SNPs result in one or a combination of the following amino acids at the given positions i) the amino acid glycine (G78) or arginine (R78) at position 78; ii) the amino acid serine (S279) or leucine (L279) at position 279; of the full-length unprocessed PILRA. In some embodiments, the SNP results in the amino acid arginine at position 78 of the full-length unprocessed PILRA. In some embodiments, the SNP is rs1859788.

In some embodiments of any of the methods, the one or more variants of PILRA comprise one or a combination of the following amino acids at the given positions i) the amino acid glycine (G78) or arginine (R78) at position 78; ii) the amino acid serine (S279) or leucine (L279) at position 279; of the full-length unprocessed PILRA. In some embodiments, the one or more variants of PILRA comprise the amino acid arginine (R78) at position 78 of the full-length unprocessed PILRA. In some embodiments, the SNP results in the amino acid arginine (R78) at position 78 of the full-length unprocessed PILRA. In some embodiments, the SNP is rs1859788.

In some embodiments of any of the methods, the agent stabilizes the non-ligand bound form of the PILRA receptor. In some embodiments, the agent reduces the inhibitory signaling in myeloid cells. In some embodiments, the agent inhibits the interaction between PILRA and any one of its ligands by binding to one or more amino acids on PILRA. In some embodiments, the one or more amino acids are located within the SA binding region of PILRA. In some embodiments, the one or more amino acids are selected from the group consisting of Y33, R126, T131, R132, Q138, W139 and Q140 of the full-length unprocessed PILRA. In some embodiments, the one or more amino acids are R126 and/or Q140 of the full-length unprocessed PILRA. In some embodiments, the agent inhibits the interaction between PILRA and any one of its ligands by at least 50% as compared to a reference level. In some embodiments, the reference level is based on the interaction between the G78 variant of PILRA and any one of its ligands. In some embodiments, the agent decreases infection of a myeloid cell during HSV-1 recurrence.

In some embodiments of any of the methods, the myeloid cell is selected from the group consisting of a blood derived myeloid cell and a CNS resident myeloid cell. In some embodiments, the blood derived myeloid cell is selected from the group consisting of monocytes, macrophages, neutrophils, basophils, eosinophils, erythrocytes, dendritic cells, megakaryocytes, platelets, and mast cells. In some embodiments, the CNS resident myeloid cell is selected from the group consisting of microglia, perivascular macrophages, meningeal macrophages, and choroid plexus macrophages. In some embodiments, the CNS resident myeloid cell is a microglia.

In some embodiments of any of the methods, the sample is selected from the group consisting of cerebrospinal fluid, blood, serum, sputum, saliva, mucosal scraping, tissue biopsy, lacrimal secretion, semen, and sweat.

In some embodiments of any of the methods, the agent inhibiting the interaction between one or more variants of PILRA and any one of its ligands is administered to a subject in combination with an additional therapeutic agent. In some embodiments, the additional therapeutic agent may exert its biological effect by the same or a similar mechanism as the agent or by an unrelated mechanism of action or by a multiplicity of related and/or unrelated mechanisms of action.

In some embodiments, the additional therapeutic agent is a biologically active substance or compound such as, for example, a known compound used in the medication of AD. Generally, the additional therapeutic agent may include neutron-transmission enhancers, psychotherapeutic drugs, acetylcholine esterase inhibitors, calcium-channel blockers, biogenic amines, benzodiazepine tranquilizers, acetylcholine synthesis, storage or release enhancers, acetylcholine postsynaptic receptor agonists, monoamine oxidase-A or -B inhibitors, N-methyl-D-aspartate glutamate receptor antagonists, non-steroidal anti-inflammatory drugs, antioxidants, and serotonergic receptor antagonists. In some embodiments, the additional therapeutic agent may comprise at least one compound selected from the group consisting of compounds against oxidative stress, anti-apoptotic compounds, metal chelators, inhibitors of DNA repair such as pirenzepin and metabolites, 3-amino-1-propanesulfonic acid (3APS), 1,3-propanedisulfonate (1,3PDS), secretase activators, [beta]- and 7-secretase inhibitors, tau proteins, neurotransmitter, /3-sheet breakers, anti-inflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine and other drugs and nutritive supplements such as, for example, vitamin B 12, cysteine, a precursor of acetylcholine, lecithin, choline, Ginkgo biloba, acyetyl-L-carnitine, idebenone, propentofylline, or a xanthine derivative. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody (e.g., a monoclonal antibody).

In some embodiments, the additional therapeutic agent is a biologically active substance or compound such as, for example, a known compound used in the medication of HSV-1. Generally, the additional therapeutic agent may include an antiviral compound. In some embodiments, the antiviral compound is selected from the group consisting of acyclovir, vidarabine, azidothymidine, ganciclovir, famciclovir, penciclovir, brivudine, cidofovir, trifluridine, and foscarnet.

In some embodiments of any of the methods, the agent is for administration subcutaneously, intravenously, intramuscularly, topically, orally, transdermally, intraperitoneally, intraorbitally, by implantation, by inhalation, intrathecally, intraventricularly, or intranasally. In some embodiments, the anti-PILRA binding agent is for administration subcutaneously. In some embodiments, the anti-PILRA binding agent is for use in a human subject.

### III. Anti-PILRA Binding Agents

Provided herein are anti-PILRA binding agents for use in any of the methods described herein, e.g., methods of treating a disease associated with myeloid cell dysfunction. In some embodiments, the agent is selected from the group consisting of an antibody (e.g. anti-PILRA antibody), a polypeptide (e.g., PILRA binding polypeptide such as fusion polypeptide), polynucleotide (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecule (e.g., small molecule binding to PILRA). In some embodiments, the agent is an antibody. In some embodiments, the antibody is a monoclonal antibody. In some embodiments, the antibody is a human, humanized, or chimeric antibody. In some embodiments, the antibody is a full length IgG1 antibody. A detailed description of anti-PILRA binding agents can be found in sections A.-E. herein below.

In some embodiments, the agent stabilizes the non-ligand bound form of the PILRA receptor. In some embodiments, the agent reduces the inhibitory signaling in myeloid cells. In some embodiments, the agent inhibits the interaction between PILRA and any one of its ligands by binding to one or more amino acids on PILRA. In some embodiments, the one or more amino acids are selected from the group consisting of G78, R78, 5279 and L279 of the full-length unprocessed PILRA. In some embodiments, the one or more amino acids are located within the SA binding region of PILRA. In some embodiments, the one or more amino acids are selected from the group consisting of Y33, R126, T131, R132, Q138, W139 and Q140 of the full-length unprocessed PILRA. In some embodiments, the one or more amino acids are R126 and/or Q140 of the full-length unprocessed PILRA.

In some embodiments, the ligand is an endogenous ligand. In some embodiments, the endogenous ligand is selected from the group consisting of APLP1, C16orf54, C4A, C4B, CLEC4G, COLEC12, DAG1, EVA1C, FceRII, IL17RA, LILRB5, LRRC15, LRRTM4, NPDC1, PIANP, and PRSS55.

In some embodiments, the ligand is an endogenous ligand. In some embodiments, the endogenous ligand is selected from the group consisting of APLP1, C16orf54, C4A, C4B, CD99, CLEC4G, COLEC12, DAG1, EVA1C, FceRII, IL17RA, LILRB5, LRRC15, LRRTM4, NPDC1, PIANP, and PRSS55.

In some embodiments, the agent decreases infection of myeloid cells during HSV-1 recurrence. In some embodiments, the ligand is an exogenous ligand. In some embodiments, the exogenous ligand is HSV-1 gB.

For example, the agent according to any of the above embodiments binds to one or more residues of one or more variants of PILRA. In some embodiments, the agent binds to one or more residues of any of the amino acid sequences selected from the group consisting of SEQ ID NO:01, SEQ ID NO:02, and SEQ ID NO:03. In some embodiments, the agent binds to one or more residues of the amino acid sequence of the G78 variant of PILRA (SEQ ID NO:01). In some embodiments, the agent binds to one or more residues of the amino acid sequence of the R78 variant of PILRA (SEQ ID NO:02, UNIPROT Q9UKJ1). In some embodiments, the binding region is located within the active site of PILRA. In some embodiments, agent binds to a specific binding region on PILRA. In some embodiments, the specific binding region on PILRA is the SA binding region of PILRA. In some embodiments, the SA binding region comprises about any of 1, 2, 3, 4, 5, 6, 7, 8, 9, and/or 10 amino acid residues of PILRA. In some embodiments, the SA binding region comprises one or more of the amino acid residues of PILRA selected from the group consisting of Y33, R126, T131, R132, Q138, W139 and Q140 of full-length unprocessed PILRA. In some embodiments, the SA binding region comprises one or more of the amino acid residues R126 and/or Q140 of the full-length unprocessed PILRA.

In some embodiments, the SA binding region comprises amino acid residues that are within about any of 10, 9, 8, 7, 6, 5, 4, 3, 2, and/or 1 angstroms (Å) of any atom of an anti-PILRA binding agent. In some embodiments, the SA binding region comprises amino acid residues that are within less than any of 10, 9, 8, 7, 6, 5, 4, 3, 2, and/or 1 Å of any atom of the agent. In some embodiments, the SA binding region comprises amino acid residues that are within between any of 10-9, 9-8, 8-7, 7-6, 6-5, 5-4, 4-3, 3-2, and/or 2-1 Å of any atom of the agent. In some embodiments, the SA binding region comprises amino acid residues that are within about any of 9.5 Å, 9 Å, 8.5 Å, 8 Å, 7.5 Å, 7 Å, 6.5 Å, 6Å, 5.5 Å, 5 Å, 4.5 Å, 4Å, 3.5 Å, 3 Å, 2.5 Å, 2 Å, 1.5 Å, and/or 1 Å of any atom of the agent. The amino acid residues of the agent that contact the SA binding region (i.e., paratope) can be determined, for example, by determining the crystal structure of the agent in complex with the SA binding region of PILRA or by performing hydrogen/deuterium exchange.

Further, the anti-PILRA binding agent according to any of the above embodiments substantially or completely inhibits the interaction between PILRA and any one of its ligands. In some embodiments, the interaction between PILRA and any one of its ligands is inhibited by at least about any of 20%, 30%, 40%, 50%, 60%, 70%, 80%, 90% and/or more as compared to a reference level. In some embodiments, the interaction between PILRA and any one of its ligands is inhibited by about any of 20%, 30%, 40%, 50%, 60%, 70%, 80%, 90% and/or more as compared to a reference level. In some embodiments, the interaction between PILRA and any one of its ligands is inhibited by between any of 20-30%, 30-40%, 40-50%, 50-60%, 60-70%, 70-80%, 80-90%, and/or 90-100% as compared to a reference level. In some embodiments, the agent inhibits the interaction between PILRA and any one of its ligands by at least 50% as compared to a reference level. In some embodiments, the reference level is based on the interaction between the G78 variant of PILRA and any one of its ligands.

The inhibition of the interaction between PILRA and any one of its ligands as compared to a reference level can be analyzed by a number of methodologies, many of which are known in the art and understood by the skilled artisan, including, but not limited to, radioactive ligand binding assays such as saturation binding, non-radioactive ligand binding assays such as surface plasmon resonance, liquid phase ligand binding assays such as immunoprecipitation, or solid phase ligand binding assays.

In some embodiments of any of the anti-PILRA binding agents, the agent (e.g. anti-PILRA antibody) has a binding affinity (dissociation constant) to PILRA of less than about any of 10⁻μM, 10⁻⁸ nM, 10⁻⁹ nM, 10⁻¹⁰ nM, 10⁻¹¹ nM, 10⁻¹² nM, and/or 10⁻¹³ nM. In some embodiments, the agent has a binding affinity to PILRA of less than any of 10⁻⁷ nM, 10⁻⁸ nM, 10⁻⁹ nM, 10⁻⁴⁰ nM, 10⁻¹¹ nM, 10⁻⁴² nM, and/or 10¹³ nM.

In some embodiments of any of the anti-PILRA binding agents, the agent (e.g. anti-PILRA antibody) has an IC₅₀ of less than about any of 1000 nM, 500 nM, 100 nM, 50 nM, 10 nM, 5 nM, 1 nM, 500 pM, 100 pM, 50 pM, 10 pM, 5 pM, and/or 1 pM. In some embodiments, the agent has an IC₅₀ of less than any of 1000 nM, 500 nM, 100 nM, 50 nM, 10 nM, 5nM, 1 nM, 500 pM, 100 pM, 50 pM, 10 pM, 5 pM, and/or 1 pM. In some embodiments, the agent has an IC₅₀ of between about any of 50 μM-1μM, 1μM-500 nM, 500 nM-100 nM, 100 nM-10 nM, 10 nM-1 nM, 1000 pM-500 pM, 500 pM-200 pM, 200 pM-150 pM, 150 pM-100 pM, 100 pM-10 pM, and/or 10 pM-1 pM.

A. Antibodies

Provided herein are isolated anti-PILRA antibodies for use in the methods described herein. In any of the above embodiments, the anti-PILRA antibody is humanized. Further, the anti-PILRA antibody according to any of the above embodiments is a monoclonal antibody, including a chimeric, humanized or human antibody. In some embodiments, the anti-PILRA antibody is an antibody fragment, e.g., a Fv, Fab, Fab′, scFv, diabody, or F(ab′)₂ fragment. In some embodiments, the anti-PILRA antibody is a full length IgG1 antibody.

### Antibody 12C6.9 and Other Embodiments

In one embodiment, an anti-PILRA antibody is provided comprising at least one, two, three, four, five, or six HVRs selected from (a) HVR-H1 comprising the amino acid sequence of SEQ ID NO:31; (b) HVR-H2 comprising the amino acid sequence of SEQ ID NO:32; (c) HVR-H3 comprising the amino acid sequence of SEQ ID NO:33; (d) HVR-L1 comprising the amino acid sequence of SEQ ID NO:28; (e) HVR-L2 comprising the amino acid sequence of SEQ ID NO:29; and (f) HVR-L3 comprising the amino acid sequence of SEQ ID NO:30.

In one embodiment, an anti-PILRA antibody is provided comprising at least one, at least two, or all three VH HVR sequences selected from (a) HVR-H1 comprising the amino acid sequence of SEQ ID NO:31; (b) HVR-H2 comprising the amino acid sequence of SEQ ID NO:32; and (c) HVR-H3 comprising the amino acid sequence of SEQ ID NO:33. In one embodiment, the antibody comprises HVR-H3 comprising the amino acid sequence of SEQ ID NO:33. In another embodiment, the antibody comprises HVR-H3 comprising the amino acid sequence of SEQ ID NO:33 and HVR-L3 comprising the amino acid sequence of SEQ ID NO:30. In a further embodiment, the antibody comprises HVR-H3 comprising the amino acid sequence of SEQ ID NO:33, HVR-L3 comprising the amino acid sequence of SEQ ID NO:30, and HVR-H2 comprising the amino acid sequence of SEQ ID NO:32. In a further embodiment, the antibody comprises (a) HVR-H1 comprising the amino acid sequence of SEQ ID NO:31; (b) HVR-H2 comprising the amino acid sequence of SEQ ID NO:32; and (c) HVR-H3 comprising the amino acid sequence of SEQ ID NO:33.

In one embodiment, an anti-PILRA antibody is provided comprising at least one, at least two, or all three VL HVR sequences selected from (a) HVR-L1 comprising the amino acid sequence of SEQ ID NO:28; (b) HVR-L2 comprising the amino acid sequence of SEQ ID NO:29; and (c) HVR-L3 comprising the amino acid sequence of SEQ ID NO:30. In one embodiment, the antibody comprises (a) HVR-L1 comprising the amino acid sequence of SEQ ID NO:28; (b) HVR-L2 comprising the amino acid sequence of SEQ ID NO:29; and (c) HVR-L3 comprising the amino acid sequence of SEQ ID NO:30.

In one embodiment, an anti-PILRA antibody is provided comprising (a) a VH domain comprising at least one, at least two, or all three VH HVR sequences selected from (i) HVR-H1 comprising the amino acid sequence of SEQ ID NO:31, (ii) HVR-H2 comprising the amino acid sequence of SEQ ID NO:32, and (iii) HVR-H3 comprising the amino acid sequence of SEQ ID NO:33; and (b) a VL domain comprising at least one, at least two, or all three VL HVR sequences selected from (i) HVR-L1 comprising the amino acid sequence of SEQ ID NO:28, (ii) HVR-L2 comprising the amino acid sequence of SEQ ID NO:29, and (c) HVR-L3 comprising the amino acid sequence of SEQ ID NO:30.

In one embodiment, an anti-PILRA antibody is provided comprising (a) HVR-H1 comprising the amino acid sequence of SEQ ID NO:31; (b) HVR-H2 comprising the amino acid sequence of SEQ ID NO:32; (c) HVR-H3 comprising the amino acid sequence of SEQ ID NO:33; (d) HVR-L1 comprising the amino acid sequence of SEQ ID NO:28; (e) HVR-L2 comprising the amino acid sequence of SEQ ID NO:29; and (f) HVR-L3 comprising the amino acid sequence of SEQ ID NO:30.

In one embodiment, an anti-PILRA antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:47. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-PILRA antibody comprising that sequence retains the ability to bind to PILRA. In certain embodiments, a total of 1 to 10 amino acids have been substituted, inserted and/or deleted in SEQ ID NO:47. In certain embodiments, substitutions, insertions, or deletions occur in regions outside the HVRs (i.e., in the FRs). Optionally, the anti-PILRA antibody comprises the VH sequence in SEQ ID NO:47, including post-translational modifications of that sequence. In a particular embodiment, the VH comprises one, two or three HVRs selected from: (a) HVR-H1 comprising the amino acid sequence of SEQ ID NO:31, (b) HVR-H2 comprising the amino acid sequence of SEQ ID NO:32, and (c) HVR-H3 comprising the amino acid sequence of SEQ ID NO:33.

In one embodiment, an anti-PILRA antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:46. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-PILRA antibody comprising that sequence retains the ability to bind to PILRA. In certain embodiments, a total of 1 to 10 amino acids have been substituted, inserted and/or deleted in SEQ ID NO:46. In certain embodiments, the substitutions, insertions, or deletions occur in regions outside the HVRs (i.e., in the FRs). Optionally, the anti-PILRA antibody comprises the VL sequence in SEQ ID NO:46, including post-translational modifications of that sequence. In a particular embodiment, the VL comprises one, two or three HVRs selected from (a) HVR-L1 comprising the amino acid sequence of SEQ ID NO:28; (b) HVR-L2 comprising the amino acid sequence of SEQ ID NO:29; and (c) HVR-L3 comprising the amino acid sequence of SEQ ID NO:30.

In one embodiment, an anti-PILRA antibody is provided, wherein the antibody comprises a VH as in any of the embodiments provided above, and a VL as in any of the embodiments provided above. In one embodiment, the antibody comprises the VH and VL sequences in SEQ ID NO:47 and SEQ ID NO:46, respectively, including post-translational modifications of those sequences.

### Antibody 12111.8 and Other Embodiments

In one embodiment, an anti-PILRA antibody is provided comprising at least one, two, three, four, five, or six HVRs selected from (a) HVR-H1 comprising the amino acid sequence of SEQ ID NO:37; (b) HVR-H2 comprising the amino acid sequence of SEQ ID NO:38; (c) HVR-H3 comprising the amino acid sequence of SEQ ID NO:39; (d) HVR-L1 comprising the amino acid sequence of SEQ ID NO:34; (e) HVR-L2 comprising the amino acid sequence of SEQ ID NO:35; and (f) HVR-L3 comprising the amino acid sequence of SEQ ID NO:36.

In one embodiment, an anti-PILRA antibody is provided comprising at least one, at least two, or all three VH HVR sequences selected from (a) HVR-H1 comprising the amino acid sequence of SEQ ID NO:37; (b) HVR-H2 comprising the amino acid sequence of SEQ ID NO:38; and (c) HVR-H3 comprising the amino acid sequence of SEQ ID NO:39. In one embodiment, the antibody comprises HVR-H3 comprising the amino acid sequence of SEQ ID NO:39. In another embodiment, the antibody comprises HVR-H3 comprising the amino acid sequence of SEQ ID NO:39 and HVR-L3 comprising the amino acid sequence of SEQ ID NO:36. In a further embodiment, the antibody comprises HVR-H3 comprising the amino acid sequence of SEQ ID NO:39, HVR-L3 comprising the amino acid sequence of SEQ ID NO:36, and HVR-H2 comprising the amino acid sequence of SEQ ID NO:38. In a further embodiment, the antibody comprises (a) HVR-H1 comprising the amino acid sequence of SEQ ID NO:37; (b) HVR-H2 comprising the amino acid sequence of SEQ ID NO:38; and (c) HVR-H3 comprising the amino acid sequence of SEQ ID NO:39.

In one embodiment, an anti-PILRA antibody is provided comprising at least one, at least two, or all three VL HVR sequences selected from (a) HVR-L1 comprising the amino acid sequence of SEQ ID NO:34; (b) HVR-L2 comprising the amino acid sequence of SEQ ID NO:35; and (c) HVR-L3 comprising the amino acid sequence of SEQ ID NO:36. In one embodiment, the antibody comprises (a) HVR-L1 comprising the amino acid sequence of SEQ ID NO:34; (b) HVR-L2 comprising the amino acid sequence of SEQ ID NO:35; and (c) HVR-L3 comprising the amino acid sequence of SEQ ID NO:36.

In one embodiment, an anti-PILRA antibody is provided comprising (a) a VH domain comprising at least one, at least two, or all three VH HVR sequences selected from (i) HVR-H1 comprising the amino acid sequence of SEQ ID NO:37, (ii) HVR-H2 comprising the amino acid sequence of SEQ ID NO:38, and (iii) HVR-H3 comprising the amino acid sequence of SEQ ID NO:39; and (b) a VL domain comprising at least one, at least two, or all three VL HVR sequences selected from (i) HVR-L1 comprising the amino acid sequence of SEQ ID NO:34, (ii) HVR-L2 comprising the amino acid sequence of SEQ ID NO:35, and (c) HVR-L3 comprising the amino acid sequence of SEQ ID NO:36.

In one embodiment, an anti-PILRA antibody is provided comprising (a) HVR-H1 comprising the amino acid sequence of SEQ ID NO:37; (b) HVR-H2 comprising the amino acid sequence of SEQ ID NO:38; (c) HVR-H3 comprising the amino acid sequence of SEQ ID NO:39; (d) HVR-L1 comprising the amino acid sequence of SEQ ID NO:34; (e) HVR-L2 comprising the amino acid sequence of SEQ ID NO:35; and (f) HVR-L3 comprising the amino acid sequence of SEQ ID NO:36.

In one embodiment, an anti-PILRA antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:49. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-PILRA antibody comprising that sequence retains the ability to bind to PILRA. In certain embodiments, a total of 1 to 10 amino acids have been substituted, inserted and/or deleted in SEQ ID NO:49. In certain embodiments, substitutions, insertions, or deletions occur in regions outside the HVRs (i.e., in the FRs). Optionally, the anti-PILRA antibody comprises the VH sequence in SEQ ID NO:49, including post-translational modifications of that sequence. In a particular embodiment, the VH comprises one, two or three HVRs selected from: (a) HVR-H1 comprising the amino acid sequence of SEQ ID NO:37, (b) HVR-H2 comprising the amino acid sequence of SEQ ID NO:38, and (c) HVR-H3 comprising the amino acid sequence of SEQ ID NO:39.

In one embodiment, an anti-PILRA antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:48. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-PILRA antibody comprising that sequence retains the ability to bind to PILRA. In certain embodiments, a total of 1 to 10 amino acids have been substituted, inserted and/or deleted in SEQ ID NO:48. In certain embodiments, the substitutions, insertions, or deletions occur in regions outside the HVRs (i.e., in the FRs). Optionally, the anti-PILRA antibody comprises the VL sequence in SEQ ID NO:48, including post-translational modifications of that sequence. In a particular embodiment, the VL comprises one, two or three HVRs selected from (a) HVR-L1 comprising the amino acid sequence of SEQ ID NO:34; (b) HVR-L2 comprising the amino acid sequence of SEQ ID NO:35; and (c) HVR-L3 comprising the amino acid sequence of SEQ ID NO:36.

In one embodiment, an anti-PILRA antibody is provided, wherein the antibody comprises a VH as in any of the embodiments provided above, and a VL as in any of the embodiments provided above. In one embodiment, the antibody comprises the VH and VL sequences in SEQ ID NO:49 and SEQ ID NO:48, respectively, including post-translational modifications of those sequences.

### Antibody 12D4 and Other Embodiments

In one embodiment, an anti-PILRA antibody is provided comprising at least one, two, three, four, five, or six HVRs selected from (a) HVR-H1 comprising the amino acid sequence of SEQ ID NO:43; (b) HVR-H2 comprising the amino acid sequence of SEQ ID NO:44; (c) HVR-H3 comprising the amino acid sequence of SEQ ID NO:45; (d) HVR-L1 comprising the amino acid sequence of SEQ ID NO:40; (e) HVR-L2 comprising the amino acid sequence of SEQ ID NO:41; and (f) HVR-L3 comprising the amino acid sequence of SEQ ID NO:42.

In one embodiment, an anti-PILRA antibody is provided comprising at least one, at least two, or all three VH HVR sequences selected from (a) HVR-H1 comprising the amino acid sequence of SEQ ID NO:43; (b) HVR-H2 comprising the amino acid sequence of SEQ ID NO:44; and (c) HVR-H3 comprising the amino acid sequence of SEQ ID NO:45. In one embodiment, the antibody comprises HVR-H3 comprising the amino acid sequence of SEQ ID NO:45. In another embodiment, the antibody comprises HVR-H3 comprising the amino acid sequence of SEQ ID NO:45 and HVR-L3 comprising the amino acid sequence of SEQ ID NO:42. In a further embodiment, the antibody comprises HVR-H3 comprising the amino acid sequence of SEQ ID NO:45, HVR-L3 comprising the amino acid sequence of SEQ ID NO:42, and HVR-H2 comprising the amino acid sequence of SEQ ID NO:44. In a further embodiment, the antibody comprises (a) HVR-H1 comprising the amino acid sequence of SEQ ID NO:43; (b) HVR-H2 comprising the amino acid sequence of SEQ ID NO:44; and (c) HVR-H3 comprising the amino acid sequence of SEQ ID NO:45.

In one embodiment, an anti-PILRA antibody is provided comprising at least one, at least two, or all three VL HVR sequences selected from (a) HVR-L1 comprising the amino acid sequence of SEQ ID NO:40; (b) HVR-L2 comprising the amino acid sequence of SEQ ID NO:41; and (c) HVR-L3 comprising the amino acid sequence of SEQ ID NO:42. In one embodiment, the antibody comprises (a) HVR-L1 comprising the amino acid sequence of SEQ ID NO:40; (b) HVR-L2 comprising the amino acid sequence of SEQ ID NO:41; and (c) HVR-L3 comprising the amino acid sequence of SEQ ID NO:42.

In one embodiment, an anti-PILRA antibody is provided comprising (a) a VH domain comprising at least one, at least two, or all three VH HVR sequences selected from (i) HVR-H1 comprising the amino acid sequence of SEQ ID NO:43, (ii) HVR-H2 comprising the amino acid sequence of SEQ ID NO:44, and (iii) HVR-H3 comprising the amino acid sequence of SEQ ID NO:45; and (b) a VL domain comprising at least one, at least two, or all three VL HVR sequences selected from (i) HVR-L1 comprising the amino acid sequence of SEQ ID NO:40, (ii) HVR-L2 comprising the amino acid sequence of SEQ ID NO:41, and (c) HVR-L3 comprising the amino acid sequence of SEQ ID NO:43.

In one embodiment, an anti-PILRA antibody is provided comprising (a) HVR-H1 comprising the amino acid sequence of SEQ ID NO:43; (b) HVR-H2 comprising the amino acid sequence of SEQ ID NO:44; (c) HVR-H3 comprising the amino acid sequence of SEQ ID NO:45; (d) HVR-L1 comprising the amino acid sequence of SEQ ID NO:40; (e) HVR-L2 comprising the amino acid sequence of SEQ ID NO:41; and (f) HVR-L3 comprising the amino acid sequence of SEQ ID NO:42.

In one embodiment, an anti-PILRA antibody is provided comprising a heavy chain variable domain (VH) sequence having at least any of 90%, 91%, 92%, 93%, 94%, 95%, 96%, 97%, 98%, 99%, or 100% sequence identity to the amino acid sequence of SEQ ID NO:51. In certain embodiments, a VH sequence having at least any of 90%, 91%, 92%, 93%, 94%, 95%, 96%, 97%, 98%, or 99% identity contains substitutions (e.g., conservative substitutions), insertions, or deletions relative to the reference sequence, but an anti-PILRA antibody comprising that sequence retains the ability to bind to PILRA. In certain embodiments, a total of 1 to 10 amino acids have been substituted, inserted and/or deleted in SEQ ID NO:51. In certain embodiments, substitutions, insertions, or deletions occur in regions outside the HVRs (i.e., in the FRs). Optionally, the anti-PILRA antibody comprises the VH sequence in SEQ ID NO:51, including post-translational modifications of that sequence. In a particular embodiment, the VH comprises one, two or three HVRs selected from: (a) HVR-H1 comprising the amino acid sequence of SEQ ID NO:43, (b) HVR-H2 comprising the amino acid sequence of SEQ ID NO:44, and (c) HVR-H3 comprising the amino acid sequence of SEQ ID NO:45.

In one embodiment, an anti-PILRA antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:50. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-PILRA antibody comprising that sequence retains the ability to bind to PILRA. In certain embodiments, a total of 1 to 10 amino acids have been substituted, inserted and/or deleted in SEQ ID NO:50. In certain embodiments, the substitutions, insertions, or deletions occur in regions outside the HVRs (i.e., in the FRs). Optionally, the anti-PILRA antibody comprises the VL sequence in SEQ ID NO:50, including post-translational modifications of that sequence. In a particular embodiment, the VL comprises one, two or three HVRs selected from (a) HVR-L1 comprising the amino acid sequence of SEQ ID NO:40; (b) HVR-L2 comprising the amino acid sequence of SEQ ID NO:41; and (c) HVR-L3 comprising the amino acid sequence of SEQ ID NO:42.

In one embodiment, an anti-PILRA antibody is provided, wherein the antibody comprises a VH as in any of the embodiments provided above, and a VL as in any of the embodiments provided above. In one embodiment, the antibody comprises the VH and VL sequences in SEQ ID NO:51 and SEQ ID NO:50, respectively, including post-translational modifications of those sequences.

In a further aspect, the anti-PILRA antibody according to any of the above embodiments may incorporate any of the features, singly or in combination, as described in Sections below:

1. Affinity

In some embodiments, the anti-PILRA antibody provided herein has a dissociation constant (Kd) of ≤1 μM, ≤100 nM, ≤10 nM, ≤1 nM, ≤0.1 nM, ≤0.01 nM, and/or ≤0.001 nM (e.g., 10⁻⁸ M or less, e.g., from 10⁻⁸ M to 10⁻¹³ M, e.g., from 10⁻⁹ M to 10⁻¹³ M). In some embodiments, Kd is measured by a radiolabeled antigen binding assay (RIA). In some embodiments, the RIA is performed with the Fab version of an anti-PILRA antibody and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for 2-5 hrs at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hrs) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for 10 min. Concentrations of each Fab that give less than or equal to 20% of maximal binding are chosen for use in competitive binding assays.

In some embodiments, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In some embodiments, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled polypeptide. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{d}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrometer (Aviv Instruments) or a 8000-series SLM-AMINCO™ spectrophotometer (ThermoSpectronic) with a stirred cuvette.

2. Antibody Fragments

In some embodiments, the anti-PILRA antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (Domantis, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

3. Chimeric and Humanized Antibodies

In some embodiments, the anti-PILRA antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat Nos. 5, 821,337, 7,527,791, 6,982,321, and 7,087,409; Kashmiri et al., Methods 36:25-34 (2005) (describing specificity-determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al.,Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In some embodiments, the anti-PILRA antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMab® technology; U.S. Pat. No. 7,041,870 describing K-M MOUSE® technology, and U.S. Patent Application Publication No. US 2007/0061900, describing VelociMouse® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Hist. & Histopath., 20(3):927-937 (2005) and Vollmers and Brandlein, Methods Find Exp. Clin. Pharmacol., 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

In some embodiments, the anti-PILRA antibody may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods Mol. Biol. 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, Methods Mol. Biol. 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In some embodiments, the anti-PILRA antibody provided herein is a multispecific antibody, e.g., a bispecific antibody. Multispecific antibodies are monoclonal antibodies that have binding specificities for at least two different sites. In some embodiments, one of the binding specificities is PILRA and the other is for any other antigen. In some embodiments, bispecific antibodies may bind to two different epitopes of PILRA. Bispecific antibodies may also be used to localize cytotoxic agents to cells which express PILRA. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to a polypeptide of interest, such as PILRA as well as another, different antigen (see, US 2008/0069820, for example).

B. PILRA Binding Polypeptides

In some embodiments, PILRA binding polypeptides are also provided for use in the methods described herein. In some embodiments, the PILRA binding polypeptide inhibits the interaction between PILRA and any one of its ligands. In some embodiments, the PILRA binding polypeptide is a fusion polypeptide.

PILRA binding polypeptides may be chemically synthesized using known polypeptide synthesis methodology or may be prepared and purified using recombinant technology. PILRA binding polypeptides are usually at least about 5 amino acids in length, alternatively at least about 10, 15, 20, 25, 30, 35, 40, 45, 50, 55, 60, 65, 70, 75, 80, 85, 90, 95, and/or 100 amino acids in length and/or more, wherein such PILRA binding polypeptides that are capable of binding, preferably specifically, to PILRA.

PILRA binding polypeptides may be identified without undue experimentation using well known techniques. In this regard, it is noted that techniques for screening polypeptide libraries for polypeptides that are capable of specifically binding to PILRA are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

C. Small Molecules Binding to PILRA

Provided herein are small molecules for use as a PILRA binding agent for use in the methods described above. In some embodiments, the small molecule binding to PILRA substantially or completely inhibits the interaction between PILRA and any one of its ligands.

Small molecules are preferably organic molecules other than polypeptides or antibodies as defined herein that bind, preferably specifically, to PILRA as described herein. Binding organic small molecules may be identified and chemically synthesized using known methodology (see, e.g., PCT Publication Nos. WO00/00823 and WO00/39585). Binding organic small molecules are usually less than about 2000 daltons in size, alternatively less than about 1500, 750, 500, 250 or 200 daltons in size, wherein such organic small molecules that are capable of binding, preferably specifically, to a polypeptide as described herein may be identified without undue experimentation using well known techniques. In this regard, it is noted that techniques for screening organic small molecule libraries for molecules that are capable of binding to a polypeptide of interest are well known in the art (see, e.g., PCT Publication Nos. WO00/00823 and WO00/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. PILRA Polynucleotide Antagonists

Provided herein are also PILRA polynucleotide antagonists for use in the methods described herein. The PILRA polynucleotide antagonist may be an antisense nucleic acid and/or a ribozyme. The antisense nucleic acids comprise a sequence complementary to at least a portion of an RNA transcript of PILRA. However, absolute complementarity, although preferred, is not required.

The PILRA polynucleotide antagonist may be a nucleic acid that hybridizes under stringent conditions to PILRA nucleic acid sequences (e.g., siRNA and CRISPR-RNA, including sgRNAs having a CRISPR-RNA and tracrRNA sequence). See Mali et al., Science. 339: 823-26, (2013).

A sequence “complementary to at least a portion of an RNA,” referred to herein, means a sequence having sufficient complementarity to be able to hybridize with the RNA, forming a stable duplex; in the case of double stranded antisense nucleic acids, a single strand of the duplex DNA may thus be tested, or triplex formation may be assayed. The ability to hybridize will depend on both the degree of complementarity and the length of the antisense nucleic acid. Generally, the larger the hybridizing nucleic acid, the more base mismatches with a RNA it may contain and still form a stable duplex (or triplex as the case may be). One skilled in the art can ascertain a tolerable degree of mismatch by use of standard procedures to determine the melting point of the hybridized complex.

Polynucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the gene, could be used in an antisense approach to inhibit translation of endogenous mRNA. Polynucleotides complementary to the 5′ untranslated region of the mRNA should include the complement of the AUG start codon. Antisense polynucleotides complementary to mRNA coding regions are less efficient inhibitors of translation. Whether designed to hybridize to the 5′-, 3′- or coding region of an mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

E. Variants of Antibodies and Binding Polypeptides Described Herein

1. Glycosylation variants

In any of the above embodiments, the antibody (e.g., anti-PILRA antibody) or the polypeptide (e.g., PILRA binding polypeptide) provided herein is altered to increase or decrease the extent to which the antibody or the polypeptide is glycosylated. Addition or deletion of glycosylation sites a polypeptide may be conveniently accomplished by altering the amino acid sequence such that one or more glycosylation sites is created or removed.

Where the antibody or polypeptide comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and SA, as well as a fucose attached to a GlcNAc in the “stem” of the biantennary oligosaccharide structure. In some embodiments, modifications of the oligosaccharide in the antibody or polypeptide as described herein may be made in order to create variants with certain improved properties.

In some embodiments, antibody or polypeptide variants are provided having a carbohydrate structure that lacks fucose attached (directly or indirectly) to an Fc region. For example, the amount of fucose in such antibody or Fc fusion polypeptide may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn 297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies or polypeptides. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al., Biotech. Bioeng. 87: 614 (2004). Examples of cell lines capable of producing defucosylated antibodies include Lec13 CHO cells deficient in polypeptide fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

2. Fc Region Variants

In some embodiments, one or more amino acid modifications may be introduced into the Fc region of the antibody (e.g., anti-PILRA antibody) or the polypeptide (e.g., PILRA binding polypeptide). The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, provided is an antibody variant or polypeptide variant that possesses some but not all effector functions, which make it a desirable candidate for applications in which the half-life of the antibody or polypeptide in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody or polypeptide lacks FcγR binding (hence likely lacking ADCC activity), but retains FcRn binding ability. The primary cells for mediating ADCC, NK cells, express Fc(RIII) only, whereas monocytes express Fc(RI), Fc(RII) and Fc(RIII). FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody or polypeptide variants with improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, an antibody variant or polypeptide variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. No. 5,648,260; U.S. Pat. No. 5,624,821; and WO 94/29351 concerning other examples of Fc region variants.

3. Cysteine Engineered Variants

In some embodiments, it may be desirable to create cysteine engineered antibody (e.g., anti-PILRA antibody) or the polypeptide (e.g., PILRA binding polypeptide), in which one or more residues are substituted with cysteine residues. In particular embodiments, the substituted residues occur at accessible sites of the antibody or the polypeptide. By substituting those residues with cysteine, reactive thiol groups are thereby positioned at accessible sites of the antibody and may be used to conjugate the antibody or the polypeptide to other moieties, such as drug moieties or linker-drug moieties, to create an immunoconjugate, as described further herein. In some embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies or Fc fusion polypeptides may be generated as described, e.g., in U.S. Pat. No. 7,521,541.

4. Amino Acid Variants Antibody Variants

In some embodiments, amino acid sequence variants of the antibody (e.g., anti-PILRA antibody) or the polypeptide (e.g., PILRA binding polypeptide) provided herein are contemplated. For example, it may be desirable to improve the binding affinity and/or other biological properties of antibody or the polypeptide. Amino acid sequence variants of the antibody or the polypeptide may be prepared by introducing appropriate modifications into the nucleotide sequence encoding the antibody or the polypeptide, or by peptide synthesis. Such modifications include, for example, deletions from, and/or insertions into and/or substitutions of residues within the amino acid sequences of the antibody or the polypeptide. Any combination of deletion, insertion, and substitution can be made to arrive at the final construct, provided that the final construct possesses the desired characteristics, e.g., antigen-binding.

In some embodiments, the antibody variants or the polypeptide variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into the antibody or the polypeptide and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

5. Derivatives

In some embodiments, the antibody (e.g., anti-PILRA antibody) or the polypeptide (e.g., PILRA binding polypeptide) provided herein can be further modified to contain additional non-proteinaceous moieties that are known in the art and readily available. The moieties suitable for derivatization of the antibody or the polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody and/or polypeptide may vary, and if more than one polymer is attached, they can be the same or different molecules. In general, the number and/or type of polymers used for derivatization can be determined based on considerations including, but not limited to, the particular properties or functions of the antibody and/or polypeptide to be improved, whether the antibody derivative and/or polypeptide derivative will be used in a therapy under defined conditions, etc.

In another embodiment, conjugates of an antibody and/or polypeptide to non-proteinaceous moiety that may be selectively heated by exposure to radiation are provided. In some embodiments, the non-proteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the non-proteinaceous moiety to a temperature at which cells proximal to the non-proteinaceous moiety are killed.

IV Pharmaceutical Formulations and Methods of Administration

Pharmaceutical formulations of the anti-PILRA binding agent as described herein are prepared by mixing such agents having the desired degree of purity with one or more optional pharmaceutically acceptable carriers in the form of lyophilized formulations or aqueous solutions. See Remington's Pharmaceutical Sciences 16th edition, Osol, A. Ed. (1980). In some embodiments, the anti-PILRA binding agents provided herein are antibodies (e.g., anti-PILRA antibodies), polypeptides (e.g., PILRA binding polypeptide), polynucleotides (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecules (e.g., small molecule binding to PILRA).

Pharmaceutically acceptable carriers are generally non-toxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. See Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the anti-PILRA binding agent which matrices are in the form of shaped articles, e.g., films, or microcapsules.

The formulations to be used for in vivo administration are generally sterile. Sterility may be readily accomplished, e.g., by filtration through sterile filtration membranes.

Further provided herein are pharmaceutical formulations comprising an anti-PILRA binding agent for use in the methods described herein. In some embodiments, the formulation comprises a pharmaceutically acceptable carrier, adjuvant, or vehicle. In some embodiments, the formulation comprises an amount of the agent effective to measurably inhibit the interaction between PILRA and any one of its ligands. In some embodiments, the formulation is formulated for administration to a subject in need thereof.

Formulations comprising an anti-PILRA binding agent may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Specific dosage and treatment regimen for any particular subject will depend upon a variety of factors, including age, body weight, general health, sex, diet, time of administration, rate of excretion, drug combination, the judgment of the treating physician, and the severity of the particular disease being treated. The amount of a provided anti-PILRA binding agent in the formulation will also depend upon the particular compound in the formulation.

In some embodiments, the effective amount of the anti-PILRA binding agent administered per dose will be in the range of about 0.01-100 mg/kg, alternatively about 0.1 to 20 mg/kg of subject body weight per day, with the typical initial range of compound used being 0.3 to 15 mg/kg/day.

The anti-PILRA binding agent may be employed alone or in combination with other agents for treatment as described above. For example, the second agent of the pharmaceutical combination formulation or dosing regimen may have complementary activities to the anti-PILRA binding agent such that they do not adversely affect each other. The compounds may be administered together in a unitary pharmaceutical formulation or separately.

The term “co-administering” refers to either simultaneous administration, or any manner of separate sequential administration, of an anti-PILRA binding agent, and a further active pharmaceutical ingredient or ingredients. If the administration is not simultaneous, the compounds are administered in a close time proximity to each other. Furthermore, it does not matter if the compounds are administered in the same dosage form, e.g., one compound may be administered topically and another compound may be administered orally. Typically, any agent that has activity against a disease or condition being treated may be co-administered. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the disease involved.

V. Methods of Screening and/or Identifying Anti-PILRA Binding Agents With Desired Function

Additional anti-PILRA binding agents for use in the methods described herein, including antibodies (e.g., anti-PILRA antibodies), polypeptides (e.g., PILRA binding polypeptides), polynucleotides (e.g., PILRA polynucleotide antagonists such as short interfering RNAs (siRNA) or clustered regularly interspaced short palindromic repeat RNAs (CRISPR-RNA or crRNA, including single guide RNAs (sgRNAs) having a crRNA and tracrRNA sequence), and small molecules (e.g., small molecule binding to PILRA) may be identified, screened for, or characterized for their physical/chemical properties and/or biological activities by various assays known in the art.

A candidate anti-PILRA binding agent may be computationally evaluated and designed by means of a series of steps in which chemical entities or fragments are screened and selected for their ability to associate with individual binding target sites on PILRA, e.g. the SA binding region. One skilled in the art may use one of several methods to screen chemical entities or fragments for their ability to associate with PILRA, and more particularly with target sites on PILRA. The process may begin by visual inspection of, for example a target site on a computer screen, based on the PILRA coordinates, or a subset of those coordinates known in the art.

In some embodiments of any of the methods of screening and/or identifying, the candidate anti-PILRA binding agent is an antibody, polypeptide, polynucleotide or small molecule binding to PILRA. In some embodiments, the agent substantially or completely inhibits the interaction between PILRA and any one of its ligands. In some embodiments, the agent binds to a specific binding region on PILRA. In some embodiments, the agent binds to the SA binding region of PILRA. In some embodiments, the SA binding region comprises one or more of the amino acid residues of PILRA selected from the group consisting of Y33, R126, T131, R132, Q138, W139 and Q140 of the full-length unprocessed PILRA. In some embodiments, the SA binding region comprises one or more of the amino acid residues of PILRA, wherein the one or more amino acids are R126 and/or Q140 of full-length unprocessed PILRA.

The antibodies, polypeptides, polynucleotides, and/or small molecules binding to PILRA provided herein may be identified, screened for, or characterized for their physical/chemical properties and/or biological activities by various assays known in the art.

In one aspect, the antibodies, polypeptides, polynucleotides and/small molecules binding to PILRA provided herein are tested for their PILRA binding activity, e.g., by known methods such as ELISA, western blotting analysis, cell surface binding by Scatchard or surface plasmon resonance. In another aspect, competition assays may be used to identify an antibody that competes with the anti-PILRA antibody or PILRA polypeptide provided herein for binding to PILRA. In a further aspect, the anti-PILRA antibody or PILRA polypeptide provided herein can be used for detecting the presence or amount of PILRA present in a biological sample. In some embodiments, the biological sample is first blocked with a non-specific isotype control antibody to saturate any Fc receptors in the sample.

In one aspect, assays are provided for identifying the biological activity of the anti-PILRA antibody or PILRA polypeptide provided herein. In some embodiments, such assays for identifying the biological activity are e.g., peptide substrate assays or coupled assays. Biological activity of the anti-PILRA antibody or PILRA polypeptide may include, e.g., binding to PILRA, and thereby inhibiting the interaction between PILRA and any one of its ligands.

### Articles of Manufacture

In another aspect, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a formulation which is by itself or combined with another formulation effective for treating, preventing and/or diagnosing the condition and may have a sterile access port (for example the container may be an intravenous solution bag or a vial having a stopper pierceable by a hypodermic injection needle). At least one active agent in the formulation is an anti-PILRA binding agent as described herein. The label or package insert indicates that the formulation is used for treating the condition of choice. Moreover, the article of manufacture may comprise (a) a first container with a formulation contained therein, wherein the formulation comprises an anti-PILRA binding agent and (b) a second container with a formulation contained therein, wherein the formulation comprises a therapy agent for treatment of AD or HSV-1 infection.

In some embodiments, the article of manufacture comprises a container, a label on said container, and a formulation contained within said container; wherein the formulation includes one or more reagents (e.g., primary antibodies, probes and/or primers), the label on the container, and instructions for using the reagents. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label.

In some embodiments of any of the article of manufacture, the anti-PILRA binding agent is an antibody, polypeptide, polynucleotide and/or a small molecule binding to PILRA as provided herein.

The article of manufacture in this embodiment may further comprise a package insert indicating that the formulations can be used to treat a particular condition. In some embodiments, the package insert comprises instructions for administering the anti-PILRA binding agent as therapy agent for treating AD or HSV-1 infection. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc.

### VI. Specific Embodiments of the Invention

The following items further provide specific aspects of the disclosure, and specific embodiments to practice the teachings provided herein.
1. A method for treating a disease associated with myeloid cell dysfunction in a subject comprising administering an effective amount of an agent to the subject, wherein the agent specifically binds to one or more variants of Paired Immunoglobulin-like Type 2 Receptor Alpha (PILRA) thereby inhibiting the interaction between PILRA and any one of its ligands.
2. A method of selecting a subject having a disease associated with myeloid cell dysfunction for a treatment with an agent inhibiting the interaction between one or more variants of PILRA and any one of its ligands, comprising determining the presence or absence of the one or more variants of PILRA in a biological sample from the subject, wherein the presence of the one or more variants of PILRA indicates that the subject is suitable for treatment with the agent.
3. A method of predicting the response of a subject having a disease associated with myeloid cell dysfunction to a treatment with an agent specifically binding to one or more variants of PILRA, the method comprising:

(a) measuring whether the agent specifically binding to the one or more variants of PILRA inhibits the interaction between PILRA and any one of its ligands as compared to a reference level, and

(b) predicting that the subject will respond to the treatment when the interaction between PILRA and any one of its ligands is inhibited as compared to the reference level and predicting that the subject will not respond to the treatment when the interaction between PILRA and any one of its ligands is not inhibited as compared to the reference level.
4. A method for detecting the presence or absence of one or more variants of PILRA indicating that a subject having a disease associated with myeloid cell dysfunction is suitable for treatment with an agent inhibiting the interaction between PILRA and any one of its ligands, comprising:

(a) contacting a sample from the subject with a reagent capable of detecting the presence or absence of the one more variants of PILRA; and

(b) determining the presence or absence of the one or more variants of PILRA, wherein the presence of the one or more variants of PILRA indicates that the subject is suitable for treatment with an agent inhibiting the interaction between PILRA and any one of its ligands.
5. A method for selecting an agent for treating a disease associated with myeloid cell dysfunction, comprising determining whether the agent inhibits the interaction between PILRA and any one of its ligands, wherein the agent that inhibits the interaction between PILRA and any one of its ligands is suitable for treating the disease associated with myeloid cell dysfunction.
6. The method of any one of embodiments 1-5, wherein the disease associated with myeloid cell dysfunction is selected from the group consisting of Alzheimer's Disease (AD) and Herpes Simplex Virus-1 (HSV-1) infection.
7. The method of any one of embodiments 1-6, wherein the myeloid cell dysfunction is associated with decreased myeloid cell activity.
8. The method of any one of embodiments 1-7, wherein the one or more variants of PILRA are encoded by a polynucleotide sequence comprising one or more SNPs.
9. The method of embodiment 8, wherein the one or more SNPs result in one or a combination of the following amino acids at the given positions:

i) the amino acid glycine or arginine at position 78;

ii) the amino acid serine or leucine at position 279; of the full-length unprocessed PILRA.
10. The method of embodiment 9, wherein the SNP results in the amino acid arginine at position 78 of the full-length unprocessed PILRA.
11. The method of embodiment 10, wherein the SNP is rs1859788.
12. The method of any one of embodiments 1-11, wherein the agent stabilizes the non-ligand bound form of the PILRA receptor.
13. The method of any one of embodiments 1-12, wherein the agent reduces the inhibitory signaling in myeloid cells.
14. The method of any one of embodiments 1-13, wherein the agent inhibits the interaction between PILRA and any one of its ligands by binding to one or more amino acids on PILRA.
15. The method of embodiment 14, wherein the one or more amino acids are located within the sialic acid (SA) binding region of PILRA.
16. The method of embodiment 15, wherein the one or more amino acids are selected from the group consisting of Y33, R126, T131, R132, Q138, W139 and Q140 of the full-length unprocessed PILRA.
17. The method of embodiment 16, wherein the one or more amino acids are R126 and/or Q140 of the full-length unprocessed PILRA.
18. The method of any one of embodiments 1-17, wherein the agent inhibits the interaction between PILRA and any one of its ligands by at least 50% as compared to a reference level.
19. The method of any one of embodiments 1-18, wherein the reference level is based on the interaction between the G78 variant of PILRA and any one of its ligands.
20. The method of any one of embodiments 1-19, wherein the agent decreases infection of a myeloid cell during HSV-1 recurrence.
21. The method of any one of embodiments 1-20, wherein the myeloid cell is a CNS resident myeloid cell.
22. The method of embodiment 21, wherein the CNS resident myeloid cell is selected from the group consisting of microglia, perivascular macrophages, meningeal macrophages, and choroid plexus macrophages.
23. The method of embodiment 22, wherein the CNS resident myeloid cell is a microglia.
24. The method of any one of embodiment 1-23, wherein the agent is selected from the group consisting of an antibody, a polypeptide, a polynucleotide, and a small molecule.
25. The method of any one of embodiments 1-24, wherein the agent is an antibody.
26. The method of embodiment 25, wherein the antibody is a monoclonal antibody.
27. The method of embodiment 26, wherein the monoclonal antibody is a human, humanized, or chimeric antibody.
28. The method of any one of embodiment 24-27, wherein the antibody is a full length IgG1 antibody.
29. The method of any one of embodiments 1-28, wherein the ligand is an endogenous ligand.
30. The method of embodiment 29, wherein the endogenous ligand is selected from the group consisting of APLP1, C16orf54, C4A, C4B, CLEC4G, COLEC12, DAG1, EVA1C, FceRII, IL17RA, LILRB5, LRRC15, LRRTM4, NPDC1, PIANP, and PRSS55.
31. The method of any one of embodiments 1-28, wherein the ligand is an exogenous ligand.
32. The method of embodiment 31, wherein the exogenous ligand is HSV-1 glycoprotein B.
33. The method of any one of embodiments 1-32, wherein the sample is selected from the group consisting of cerebrospinal fluid, blood, serum, sputum, saliva, mucosal scraping, tissue biopsy, lacrimal secretion, semen, and sweat.
34. The method of any one of embodiments 1-33, wherein the subject is a human.
35. An agent specifically binding to one or more variants of PILRA for use in medical treatment or diagnosis including therapy and/or treating of a disease associated with myeloid cell dysfunction.
36. The agent of embodiment 35, wherein the agent stabilizes the non-ligand bound form of the PILRA receptor.
37. The agent of embodiment 35 or 36, wherein the agent reduces the inhibitory signaling in myeloid cells.
38. The agent of any one of embodiments 35-37, wherein the agent inhibits the interaction between the one or more variants of PILRA and any one of its ligands by binding to one or more amino acids on PILRA.
39. The agent of embodiment 38, wherein the one or more amino acids are located within the SA binding region of PILRA.
40. The agent of embodiment 39, wherein the one or more amino acids are selected from the group consisting of Y33, R126, T131, R132, Q138, W139 and Q140 of the full-length unprocessed PILRA.
41. The agent of embodiment 40, wherein the one or more amino acids are R126 and/or Q140 of the full-length unprocessed PILRA.
42. The agent of any one of embodiments 35-41, wherein the agent inhibits the interaction between the one or more variants of PILRA and any one of its ligands by at least 50% as compared to a reference level.
43. The agent of embodiment 42, wherein the reference level is based on the interaction between the G78 variant of PILRA and any one of its ligands.
44. The agent of any one of embodiments 35-43, wherein the agent decreases infection of a myeloid cell during HSV-1 recurrence.
45. The agent of any one of embodiments 35-44, wherein the myeloid cell is a CNS resident myeloid cell.
46. The agent of embodiment 45, wherein the CNS resident myeloid cell is selected from the group consisting of microglia, perivascular macrophages, meningeal macrophages, and choroid plexus macrophages.
47. The agent of embodiment 46, wherein the CNS resident myeloid cell is a microglia.
48. The agent of any one of embodiments 35-47, wherein the agent is selected from the group consisting of an antibody, a polypeptide, polynucleotide, and a small molecule.
49. The agent of any one of embodiments 35-48, wherein the agent is an antibody.
50. The agent of embodiment 49, wherein the antibody is a monoclonal antibody.
51. The agent of embodiment 50, wherein the monoclonal antibody is a human, humanized, or chimeric antibody.
52. The agent of any one of embodiments 48-51, wherein the antibody is a full length IgG1 antibody.
53. The agent of any one of embodiments 35-52, wherein the disease associated with myeloid cell dysfunction is selected from the group consisting of Alzheimer's Disease (AD) and Herpes Simplex Virus-1 (HSV-1) infection.
54. A pharmaceutical formulation comprising a pharmaceutically active amount of an agent specifically binding to one or more variants of PILRA according to any one of embodiments 35-53 and a pharmaceutically acceptable carrier.

### EXAMPLES

The following are examples of methods. It is understood that various other embodiments may be practiced, given the general description provided above. The use of any and all examples, or exemplary language (e.g., “such as”) provided herein, is intended merely to better illuminate embodiments and does not necessarily impose any limitations unless otherwise specifically recited in the claims. All documents cited herein are incorporated by reference in their entirety.

### Example 1

### Materials and Methods

PILRA variants and PILRA ligand expression and purification were performed as follows. The coding sequences (CDS) of full-length PILRA (AJ400841), human herpesvirus 1 strain KOSc glycoprotein B (HSV-1 gB) (EF157316), and neural proliferation, differentiation and control 1 (NPDC1) (NM_015392.3) were cloned in the pRK neo expression vector. Several PILRA point mutations were generated, including A72, A76, R78, G80, A140 and A141. The PILRA variants were incorporated into a full-length G78 variant of PILRA construct by site-directed mutagenesis as per the manufacturer's recommendation (Agilent Cat. No. 200523) and sequences were verified. A full length myc-DDK tagged PIANP construct was purchased from Origene (Cat. No. RC207868). Full length complement component 4A (Rodgers blood group) C4A (NM_007293.2), extra cellular domain (ECD) of amyloid beta precursor like protein 1 (APLP1) (NM_005166) (1-580 aa) and ECD of sortilin-related VPS10 domain-containing receptor 1 (SORCS1) (NM_052918) (1-1102 aa) were fused with C-terminal gD tag (US6/gD, partial Human alphaherpesvirus 1) (AAP32019.1) and GPI anchor in pRK vector. The ECD of all PILRA variants (1-196 aa) and NPDC1 (1-190 aa) were PCR amplified and cloned with C-terminal murine IgG2a Fc tag in a pRK expression vector.

ECDs of PILRA variants (G78, A72, A76, R78, G80, A140 and A141) and NPDC1 fused to the Fc region of murine IgG2a were expressed in a CHO cell expression system, supernatants collected, protein A/G affinity-purified and verified by SDS-PAGE and mass spectroscopy.

Relative PILRA-ligand binding to PILRA variant transfected cells was performed as follows. 293T cells were transfected with lipofectamine LTX reagent (ThermoFisher) with various full-length constructs of PILRA variants (G78, A72, A76, R78, G80, A140 and A141). After 48 hrs, the transfected cells were harvested and incubated with soluble mIgG2a-tagged ligand, NPDC1-mFc at 50 μg/ml (as described above) for 30 min on ice. Cells were then washed and stained with 1 μg/ml chimeric anti-PILRA antibody (mouse Fc region is substituted to human IgG1 backbone on anti-PILRA antibodies) on ice for 30 min followed by APC-conjugated mouse anti-human IgG (BD Pharmingen Cat. No. 550931) and FITC anti-mouse IgG2a (BD Pharmingen Cat. No. 553390) secondary antibodies according to manufacturer's instruction. PILRA-transfected 293T cells were examined by flow cytometry for binding of NPDC1 by measuring the frequency of APC and FITC double-positive cells. Double positive cells were gated on the G78 sample and then the gates were overlaid on subsequent samples to maintain the same cell population throughout the experiment. For each PILRA variant, the mean percentage of the number of cells binding to NPDC1-mFC relative to the G78 variant was calculated.

In the inverse experiment, 293T cells were transfected with lipofectamine LTX reagent (ThermoFisher) with known full-length PILRA ligand (NPDC1, HSV-1gB and PIANP) and predicted ligand constructs (SORCS1, APLP1 and C4A) (described above). After 48 hrs, the transfected cells were harvested and incubated with soluble mIgG2a-tagged variants of PILRA (G78, A72, A76, R78, G80) (described above) 50 μg/ml for 30 min on ice. Cells were then washed and stained with FITC anti-mouse IgG2a (BD Pharmingen Cat. No. 553390) secondary antibody according to manufacturer's instruction. PILRA ligand-transfected 293T cells were examined by flow cytometry for binding to PILRA variants by measuring the frequency of FITC-positive cells. The percentage of mean fluorescence intensity (MFI) of PILRA-mFC binding on ligand-transfected cells relative to the G78 variant of PILRA binding for each experiment was calculated.

PILRA variant ligand binding Surface Plasmon Resonance (SPR) was performed as follows. Binding of human NPDC1.Fc to PILRA-Fc variants was measured by SPR using a ProteOn XPR36 (Bio-Rad). PILRA-Fc G78 and variants (R78 and A140) were immobilized on a ProteOn GLC sensor chip (Bio-Rad) by EDC/NHS amine coupling (2000-2400 RU's) and the chip surface was deactivated by ethanolamine after immobilization. NPDC1-Fc diluted in PBST or a control Fc-tagged protein was injected at a concentration of 100 nM over the immobilized PILRA proteins at room temperature.

Isolation and differentiation of monocytes was performed as follows. Healthy human volunteers were genotyped for rs1859788 (R78 variant of PILRA) using custom design ABI SNP genotyping assay with the following primers; Forward primer seq: 5′-GCGGCCTTGTGCTGTAGAA-3′ (SEQ ID NO:21), Reverse primer seq: 5′-GCTCCCGACGTGAGAATATCC-3′ (SEQ ID NO:22), Reporter 1 sequence: VIC-ACTTCCACGGGCAGTC-NFQ (SEQ ID NO:23), Reporter 2 sequence: FAM-ACTTCCACAGGCAGTC-NFQ (SEQ ID NO:24). To control for a possible effect of the eQTL for PILRB, all volunteers selected were homozygous AA (lower PILRB expression) for rs6955367 (http://biorxiv.org/content/early/2016/09/09/074450). Genotype for rs6955367 was determined using an InfiniumOmni2.5Exome-8v1-2_A.bpm. Peripheral Blood Mononuclear Cells (PBMC's) were obtained by Ficol gradient from five pairs of homozygous donors for rs1859788 (one with each genotype AA/GG). The pairs of samples were matched for age [±5 years], gender and self-reported ethnicity. Monocytes were purified from PBMC's by negative selection using the EasySep™ Human Monocyte Enrichment Kit without CD16 Depletion (19058), as recommended by the manufacturer. Isolated monocytes were differentiated into macrophages in DMEM+10% FBS+1×glutaMax and 100 ng/ml MCSF media for 7-10 days.

HSV-1 Infection of Macrophages was performed as follows. Macrophages differentiated from healthy human monocytes were incubated with 10, 1, 0.1 and 0.01 multiplicity of infection (MOI) of HSV-1 virus at 37° C. for 1 hour with gentle swirling to allow virus adsorption. Cells were washed after 1 hr of adsorption and infection was continued for 6, 18 and 36 hrs. Supernatant was harvested at 6, 18 and 36 hrs of infection and cell debris were removed by centrifugation at 3000 rpm for 5 min at 4° C. DNA was isolated from infected cells using the QIAamp DNA mini-kit (Qiagen Cat. No. 51304). Additional cells were fixed with 4% paraformaldehyde after infection and stained with DAPI for microscopy.

The Lactate Dehyrogenase (LDH) Cytotoxicity Assay was performed as follows. The CytoTox 96® Non-Radioactive Cytotoxicity Assay (Promega Cat. No. E1780) was performed on supernatant harvested from HSV-1-infected human macrophages as per manufacturer's recommendations to measure cell toxicity after HSV-1 infection. For each sample, the percent cytotoxicity was calculated as the ratio of LDH released in culture supernatant after infection to completely lysed cells (maximum LDH release).

Quantitative Polymerase Chain Reaction was performed as follows. HSV-1 DNA was quantitated using a custom design ABI TaqMan gene expression assay, with the following primers: Forward primer seq: 5′-GGCCTGGCTATCCGGAGA-3′ (SEQ ID NO:25), Reverse primer seq: 5′-GCGCAGAGACATCGCGA-3′ (SEQ ID NO:26), HSV-1 probe: 5′-FAM-CAGCACACGACTTGGCGTTCTGTGT-MGB-3′ (SEQ ID NO:27). GAPDH DNA was quantitated using ABI endogenous control (Applied Biosystem Cat. No. 4352934E). Amplification reactions were carried out with 5 μL of extracted DNA from infected cells in a final volume of 25 μl with TaqMan Universal PCR Master Mix (Applied Biosystems Cat. No. 4304437) as per manufacturer's recommendations. HSV-1 DNA (Ct values) was normalized to cell GAPDH (Ct values) to account for cell number.

The HSV-1 Plaque Assay was performed as follows. Virus titers from HSV-1-infected cells were determined following a standard plaque assay protocol. In brief, the plaque assay was performed using Vero cells (African Green Monkey Cells) seeded at 1×10⁵ cells per well in 48-well plates. After overnight incubation at 37° C., the monolayer was ˜90-100% confluent. Supernatants harvested from HSV-1-infected human macrophages were clarified from cells and debris by centrifugation at 3000 rpm for 5 min at 4° C. Virus-containing supernatants were then diluted from 10⁻¹ to 10⁻⁸ in DMEM (1 ml total volume). Growth media was removed from Vero cells and 250 μl of supernatant dilution was transferred onto the cells, followed by incubation at 37° C. for 2 hrs with gentle swirling every 30 min to allow virus adsorption, after which the virus-containing media was aspirated. The cells were then overlaid with 2% methylcellulose containing 2× DMEM and 5% FBS and incubated at 37° C. 48 hrs post-infection, plaques were enumerated from each dilution. Virus titers were calculated in pfu/ml.

293-PILRA stable cells were generated by transfecting 293 cells with a plasmid to express mouse PILRA extracellular domain (ECD), human CD3 zeta chain transmembrane and intracellular domains. The plasmid encoded a neomycin resistance gene which confers resistance to G418. Cells stably expressing mouse PILRA extracellular domain (ECD), human CD3 zeta chain transmembrane and intracellular domains were selected using G418. Anti-mouse PILRA antibodies in mouse IgG2a format, mouse PILRA ECD, mouse PILRA ligand CD99 fused to human IgG1 Fc (CD99-Fc) and mouse PILRA ligand C12orfC53 fused to human IgG1 Fc (C12orf53-Fc) were prepared at Genentech, Inc.

The PILRA ECD-based competitive ELISA was performed as follows. MaxiSorp 384-well microwell plates (Thermo Scientific Nunc, catalog number 464718) were coated overnight at 4° C. with 2 μg/ml Neutravidin (Thermo Scientific Nunc, catalog number 31000) in 50 mM carbonate buffer, pH 9.6 at 25 μl/well, and washed with 0.05% polysorbate 20 in PBS (pH 7.4). Plates were blocked with 0.5% bovine serum albumin, 15 ppm Proclin 300 (Supelco, Bellefonte, Pa.) in PBS (80 μl/well) at room temperature for 1 h and washed. Biotinylated mouse PILRA ECD at 0.25 μg/ml (Genentech, Inc.) in 0.5% bovine serum albumin, 0.05% polysorbate 20, 15 ppm Proclin 300 in PBS, pH 7.4 (assay buffer) was added to the plates. After 1.5 hrs incubation, plates were washed. Anti-mouse PILRA antibodies were serially diluted in assay buffer and mixed with equal volume of mouse PILRA ligand mouse CD99-Fc (Genentech, Inc.) at 600 ng/ml or mouse C12orf53-Fc (Genentech, Inc.) at 300 ng/ml in assay buffer. The mixture of the serially diluted antibody and ligand-Fc was added to the plates at 25 μl/well. After a 2 hrs incubation, plates were washed. The ligand-Fc bound to the plates was detected by adding horseradish peroxidase conjugated goat anti-human IgG-Fc (Southern Bio, catalog number 2014-05). After a 1 hr incubation, plates were washed and the substrate 3,3′,5,5′-tetramethyl benzidine (Moss Inc., TMBE-1000) was added. The reaction was stopped by adding 1 M phosphoric acid. The absorbance was read at 450 nm using a microplate reader (Multiskan Ascent, Thermo Scientific, Waltham, Mass.). The titration curves were plotted using KaleidaGraph (Synerg Software, Reading, Pa.).

The 293-PILRA cell based competitive ELISA was performed as follows. 293-PILRA cells were trypsinized and seeded into U-bottom 96-well plates (Greiner Bio-one, catalog number 650185) at 0.4×10⁵ cells/well. Anti-mouse PILRA antibodies were serially diluted in 1% bovine serum albumin in PBS (sample buffer) and mixed with equal volume of mouse CD99-Fc (Genentech, Inc.) at 600 ng/ml or mouse C12orf53-Fc (Genentech, Inc.) at 300 ng/ml in sample buffer. After plates were centrifuged at 1200 rpm for 5 min at 4° C., the supernatant was decanted and the mixture of the serially diluted antibody and the ligand-Fc was added. Plates were incubated at 4° C. for 1 h with gentle shaking. Cells were washed with ice-cold 0.1% BSA in PBS, pH 7.4 (wash buffer) by centrifuging the plate at 1200 rpm for 5 min at 4° C. and the cells were resuspended in 200 μl wash buffer. After 3 times wash, cells were resuspended in 100 μl of horseradish peroxidase conjugated goat anti-human IgG-Fc (Southern Bio, catalog number 2014-05) in sample buffer. Plates were incubated at 4° C. for 1 hr with gentle shaking. Cells were washed with ice-cold wash buffer 4 times. Cells were resuspended in 100 μl of the substrate 3,3′,5,5′-tetramethyl benzidine (Moss Inc., TMBE-1000) and plates were incubated at room temperature for approximately 20 min for color development. The reaction was stopped by adding 1 M phosphoric acid. The absorbance was read at 450 nm using a microplate reader (Multiskan Ascent, Thermo Scientific, Waltham, Mass.). The titration curves were plotted using KaleidaGraph (Synerg Software, Reading, Pa.).

Anti-murine PILRA antibodies were developed using standard hybridoma development methods. Knockout mice were immunized with murine PILRA protein (Genentech, Inc.) via footpad injections every 3-4 days. Post immunization series lymph nodes and spleen were harvested and fused with SP20 cells to generate hybridomas. IgG positive/antigen positive colonies were picked using ClonepixFL methods (Molecular Devices) and cultured for 7 days in 96-well plates. Supernatants from hybridomas were screened by ELISA for binding to murine PILRA. Antigen positive hybridomas were scaled up, the supernatant was purified using Mab Select SuRe (GE Healthcare) and IgGs were further characterized. Sequences were obtained via standard molecular cloning methods and clones were recombinantly expressed using CHO cells.

Murine PILRA ligand blocking using SPRA was performed as follows. 96×96 array-based SPR imaging system (Carterra USA) was used to epitope bin a panel of monoclonal antibodies. Purified antibodies were diluted at 10 μg/ml in 10 mM sodium acetate buffer pH 4.5. Using amine coupling, antibodies were directly immobilized onto a SPR sensorprism CMD 200M chip (XanTec Bioanalytics, Germany) using a Continuous Flow Microspotter (Carterra, USA) to create an array of 96 antibodies. For binning analysis, the IBIS MX96 SPRi (Carterra USA) was used to evaluate binding to the immobilized antibodies. Murine PILRA-His (Genentech Inc.) was first injected for 4 min at 100 nM and was followed by a second 4 min injection of purified antibody or ligand at 10 μg/ml. The surface was regenerated between cycles with 10 mM Glycine pH 1.7. The experiment was performed at 25° C. in a running buffer of 10 mM HEPES, pH 7.4, 150 mM NaCl, 3 mM EDTA, and 0.005% Tween 20. The binding data was processed using Epitope Binning software tool (Carterra, Inc).

### Example 2

### R78 Variant of PILRA is Associated With Protection From AD

In previous studies, it was shown that the single variant with the lowest P value in a region discovered by GWAS is rarely the causal variant, but rather identifies a group of variants in strong linkage disequilibrium that may contain the risk-modifying variant(s). The risk-modifying haplotype at the 7q21 locus was sought to be defined, where the index variant is rs1476679 (meta P value=5.6×10⁻¹⁰, odds ratio=0.91) and a definitive causal risk variant has not been identified so far (see e.g., Lambert et al., Nat. Genet. 45, 1452-8 (2013)). In addition to disease risk, rs1476679 has been previously associated with age of onset and the risk allele of rs1476679 was associated with increased neuritic plaque and neurofibrillary tangles (see e.g., Desikan et al., PLoS Med. 14, e1002258 (2017)). Furthermore, rs1859788 is known to encode the missense allele (R78) in PILRA. In the present application, a cohort of 1,357 samples of European ancestry was used for whole genome sequencing to 30× average read-depth. These data confirmed the strong linkage between rs1476679 (intron of ZCWPW1) and rs1859788 (R78, PILRA) (see Table 2).

TABLE 2

| | Chr: position | | r² to rs1476679 | | |
|---|---|---|---|---|---|
| Variant | (HG19) | Annotation | CEU | GBR | GNE |
| rs34919929 | 7: 100,012,334 | Intronic | 1 | 1 | 1 |
| | | (ZCWPW1) | | | |
| rs60738304 | 7: 100,012,579 | Intronic | 1 | 1 | 0.97 |
| | | (ZCWPW1) | | | |
| rs34995835 | 7: 99,990,364 | Intronic | 0.98 | 1 | 0.97 |
| | | (PILRA) | | | |
| rs1859788 | 7: 99,971,834 | R78 (PILRA) | 0.93 | 0.93 | 0.89 |
| rs2405442 | 7: 99,971,313 | Synonymous | 0.93 | 0.95 | 0.88 |
| | | (PILRA) | | | |
| rs2906657 | 7: 99,984,089 | Intronic | 0.91 | 0.87 | NA |
| | | (PILRA) | | | |

Variants in Linkage Disequilibrium (r² > 0.90) with rs1476679 in European ancestry samples. Data from CEU, GBR populations are derived from 1000 Genomes project Phase 3 data. GNE = 30X whole genome sequenced samples of European ancestry from Genentech clinical trials.

In the present application, it was hypothesized that R78 variant of PILRA was the functional variant that accounts for the observed protection from AD-risk. As expected from the strong linkage disequilibrium between the R78 variant of PILRA and rs1476679 (FIG. 1A), conditional analysis demonstrated that the 2 variants were indistinguishable for AD-risk. It is known that the frequency of the R78 variant of PILRA varies considerably in world populations. The R78 variant ranges from ˜10% in African populations and 38% in European populations to 65% in East Asian populations (see e.g., Auton et al., Nature. 526, 68-74 (2015)).

Paired activating/inhibitory receptors are common in the immune system, with the activating receptor typically having weaker affinity than the inhibitory receptor toward the ligands. PILRA and PILRB are type I transmembrane proteins with highly similar extracellular domains that bind certain O-glycosylated proteins, but they differ in their intracellular signaling domains. PILRA contains an immunoreceptor tyrosine-based inhibitory motif (ITIM) (FIG. 1B), while PILRB signals through interaction with DAP12, which contains an immunoreceptor tyrosine-based activation motif (ITAM). Analysis of PILRA knockout mice suggested that PILRA plays a negative regulatory role in the inflammatory process in myeloid cells, likely by complex mechanisms under different inflammatory cues.

### Example 3

### R78 Variant of PILRA Reduces Ligand Binding

It is known that PILRA binds multiple endogenous (including COLEC12, NPDC1, CLEC4G, and PIANP) and exogenous ligands (HSV-1 gB, Streptococcus aureus derived proteins) and that optimal interaction with PILRA requires ligands to have both an O-glycosylated threonine and a specific protein motif (see e.g., Sun et al., J. Biol. Chem. 287, 15837-50 (2012)). Because the R78 variant of PILRA resides close to the SA binding region of PILRA, the present application hypothesizes that the glycine (uncharged, short amino acid) to arginine (basic, long side chain amino acid) substitution at position 78 might interfere with PILRA ligand-binding activity. To address this question, various amino acid point variants were generated around the SA binding region of PILRA. A residue conserved among PILR proteins and related SIGLEC receptors, R126 in PILRA was previously shown to be essential for SA interaction. Based on their location in the crystal structure, evolutionary conservation, and reduced binding to HSV-1 gB, amino acids R72 and F76 were predicted previously to be critical for ligand binding and were substituted to alanine as positive controls for loss-of-function. In addition, a residue (S80) outside of the SA binding region and expected to have little effect on ligand binding was substituted to glycine. The A72, A76, or G80 variants have not been detected in human populations (dbSNP v147).

To study the receptor-ligand binding, 293T cells were transfected with various PILRA variants and incubated them with purified NPDC1-mIgG2a protein (FIG. 1B). Cells were washed and NPDC1 and PILRA cell surface staining was assessed by flow cytometry using anti-mIgG2a-FITC and anti-PILRA antibodies, respectively. Expression of all the PILRA variants was confirmed and expression level was comparable to or greater than of the G78 variant of PILRA. Binding of the G78 variant of PILRA to NPDC1 was considered 100%. Both A72 and A76 mutations severely impaired NPDC1 binding (˜20% of G78 variant, p-value <0.0001). The R78 variant also showed impaired ligand binding, though to a lesser degree (˜35% of G78 variant, p<0.0001), while the G80 mutant was the least affected (˜60% of G78 variant, p<0.0001) (FIG. 1C). To further test the hypothesis that R78 variant of PILRA impacts functional ligand binding, NPDC1 or alternative PILRA ligands HSV-1 gB and PIANP were expressed on the cell surface of 293T cells, and the binding of purified PILRA protein variants was measured by flow cytometry. NPDC1, HSV-1 gB and PIANP expression was confirmed, with increased binding of the G78 variant of PILRA to cells expressing these ligands, and a consistent reduction in binding was again observed for the R78 variant of PILRA (FIG. 1D-G). These data suggested that the R78 variant impairs the functional ligand-binding activity of PILRA.

### Example 4

### R78 Variant of PILRA Stabilizes the Closed (Ligand Unbound) Form

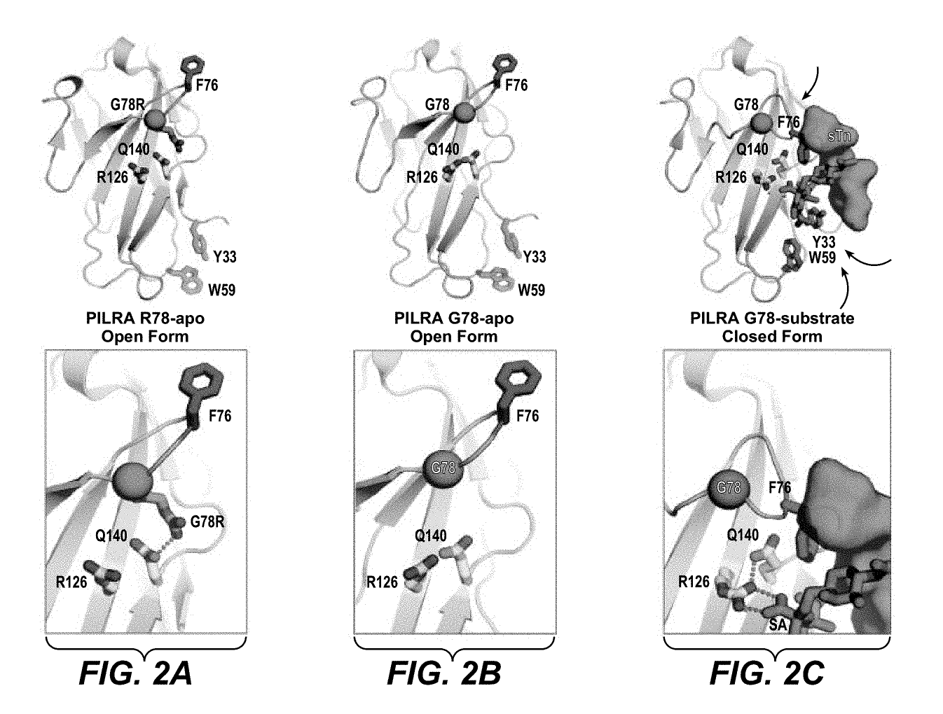

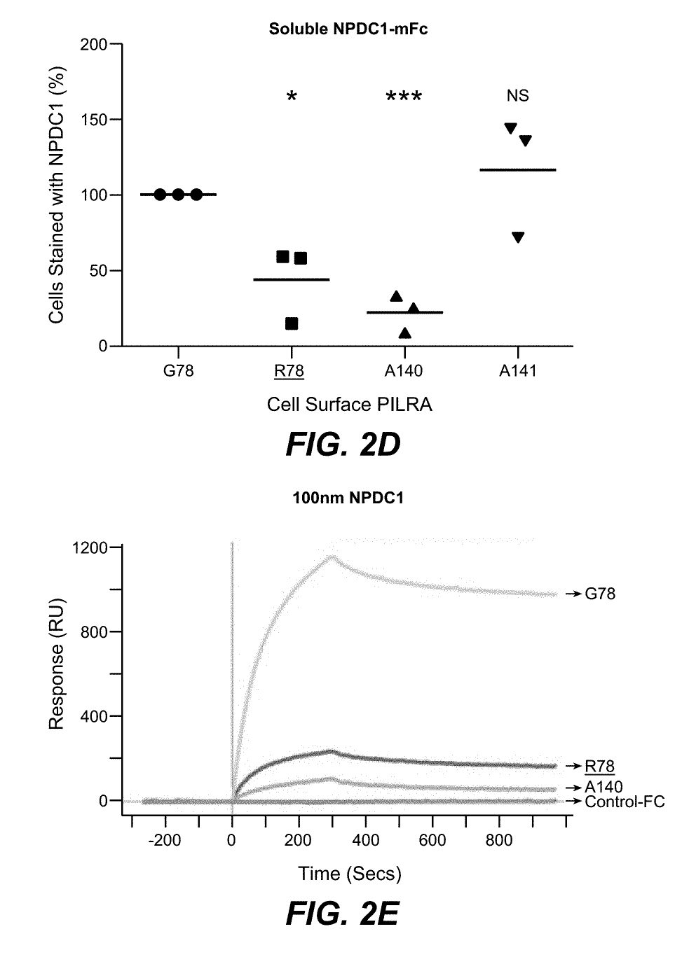

To understand the conformational changes that might occur in the PILRA SA binding region during receptor-ligand interactions in the presence of the G78 variant (AD-risk) or R78 (AD-protective) variant, publicly available experimental crystal structures were evaluated (FIG. 2A-C, see e.g. Kuroki et al., Proc. Natl. Acad. Sci. U.S.A. 111, 8877-8882 (2014), and Lu et al., Proc. Natl. Acad. Sci. U.S.A. 111, 8221-8226 (2014)). Structures of the G78 variant of PILRA reveal a monomeric extracellular domain with a single V-set Ig-like β-sandwich fold that binds O-ligands. Reminiscent of a molecular clamp, PILRA undergoes a large structural rearrangement from an “open” to a “closed” form to bind peptide and sugar moieties simultaneously. The essential R126 residue engages the carboxyl group of SA directly in a strong salt bridge (FIG. 2C). The CC′ loop contains F76 and G78 and undergoes a large conformational change where F76 translates ˜15 Å to participate in a key interaction with the peptide of the ligand and abut the Q140 side-chain of PILRA. In this ligand-bound “closed” conformation, Q140 helps to position R126 precisely for its interaction with SA. Notably, in the structure of the R78 variant of PILRA crystallized in the absence of any ligand, the long side-chain of R78 is observed to sneak down from the CC′ loop to hydrogen bond with Q140 directly (FIG. 2A). This R78 interaction has three important consequences: 1) it may alter CC′ loop dynamics, 2) it sterically hinders F76 from obtaining a ligand-bound “closed” conformation, and 3) it affects the ability of R126 to interact with the carboxyl group of SA by altering the R126-Q140 interactions typically observed in the G78 variant of PILRA. Overall, the structure of the R78 variant of PILRA implies that this single side-chain alteration may stabilize the “open” or apo form of PILRA and/or alter the conformational sampling of the molecular clamp to obtain its “closed” form and engage its ligands.

It is therefore proposed that in the G78 variant of PILRA (AD-risk), the engagement of SA by R126 and peptide by F76 is unaffected by the G78 variant (FIG. 2C). However, in the AD-protective PILRA variant R78, the R78 side-chain competes with the central R126-Q140 interaction and alters the positioning of F76 (FIG. 2A), which leads to an overall decrease in PILRA ligand binding. This structure-based hypothesis is consistent with the reduced functional cellular binding observed for the R78 variant of PILRA (FIG. 1).

To further test this model, two more alanine mutants of PILRA were generated at amino acids predicted to be essential (Q140) or non-essential (S141) for conformational changes associated with ligand interaction. 293T cells were transfected with G78, R78, A140 and A141 variants of PILRA, and receptor-ligand interaction was measured after incubating cells with soluble NPDC1-mIgG2a. PILRA expression was comparable among variants, matching or exceeding G78 expression. R78 (44% of G78, p=0.02) and A140 (22% of G78, p=0.0004) showed significantly decreased binding to NPDC1, while S141A (117%, p=0.5) had no significant effect (FIG. 2D). These data are consistent with the experimental structural models that show the interaction of Q140 with R126 is important for productive SA binding (FIG. 2A-C). Consistently, the A140 mutation has a strong effect because the Q140-R126 interaction network is completely abolished. By contrast, the AD-protective R78 variant has an intermediate effect since it only modulates the Q140 interaction with R126, which is expected to only alter the frequency or strength of relevant PILRA-ligand interactions.

### Example 5

### R78 Variant of PILRA Reduces the On-Rate of Ligand Binding

Next, the interaction of PILRA variants and ligands in vitro using surface plasmon resonance (SPR) was investigated. Human PILRA-Fc variants (G78, R78 and A140) were immobilized on a ProteOn GLC sensor chip and NPDC1-mFc or a control mFc-tagged protein were injected over immobilized PILRA protein. Qualitatively, NPDC1-Fc bound to the variants R78 (AD-protective) and A140 (essential for R126 conformation) to a much lesser extent than the G78 variant of PILRA, while control Fc-tagged protein showed no binding (FIG. 2E). To further probe the mechanistic basis of R78 function and phenotype, a more complete SPR characterization of NPDC1-HIS binding to G78 (16.8 nM) and R78 (76.5 nM) PILRA was performed. In addition to a ˜4.5-fold lower affinity, we note that the on-rate constant kₒₙ for NPDC1-His binding to R78 (6.8×10⁺³ M⁻¹s⁻¹) is ˜3-fold lower than when binding to the G78 variant of PILRA (2.2×10⁺⁴ M⁻¹s⁻¹), while the k_{off} rate constants are comparable (Table 3). These results are consistent with the idea that, once engaged, the affinity and disassociation rate of R78-ligand complexes are similar to G78 variant of PILRA, but the frequency with which PILRA can productively engage with ligands is reduced in the R78 variant by R78 side chain interactions favoring the apo-state. Taken together, our functional cell surface binding and SPR experiments support a structural model in which R78 impairs PILRA-ligand interactions by altering the accessibility of a productive SA binding conformation in PILRA.

TABLE 3

| PILRA | Affinity (nM) | On-rate (1/Ms) | Off-rate (1/Ms) |
|---|---|---|---|
| PILRA-G78 | 16.8 | 2.20E+04 | 3.70E−04 |
| PILRA-G80 | 30.0 | 1.70E+04 | 5.10E−04 |
| PILRA-R78 | 76.5 | 6.80E+03 | 5.20E−04 |

### Example 6

R78 variant of PILRA reduces entry of HSV-1 into hMDMs

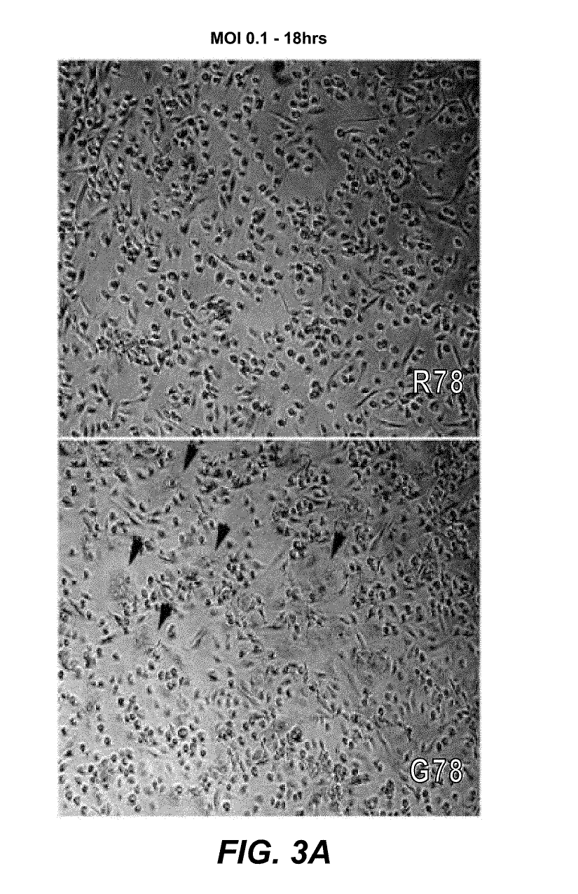

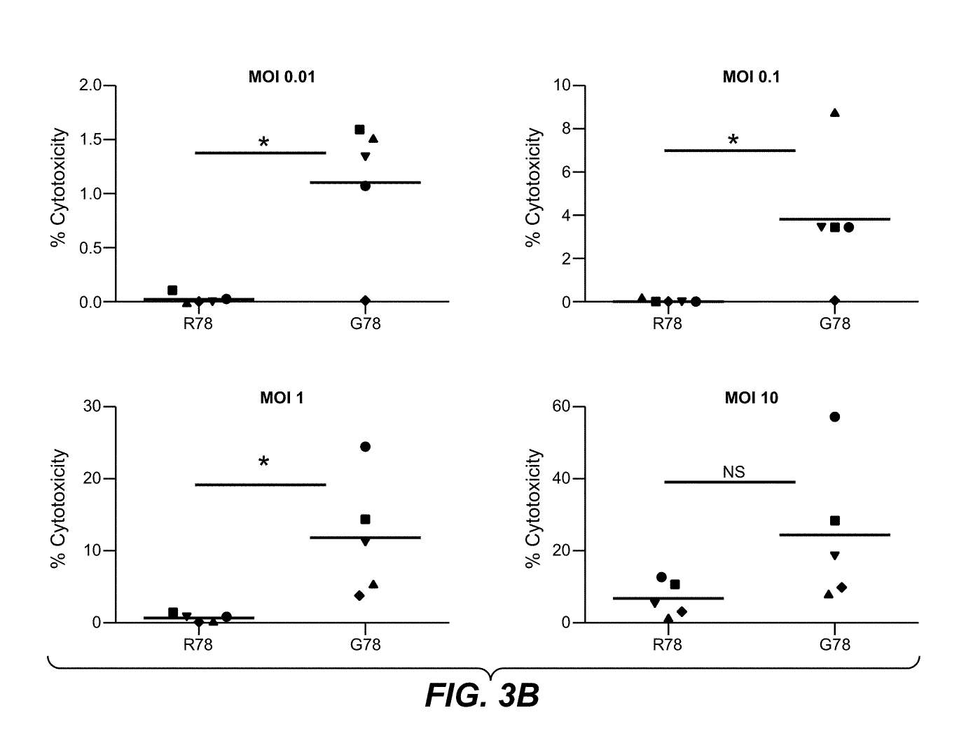

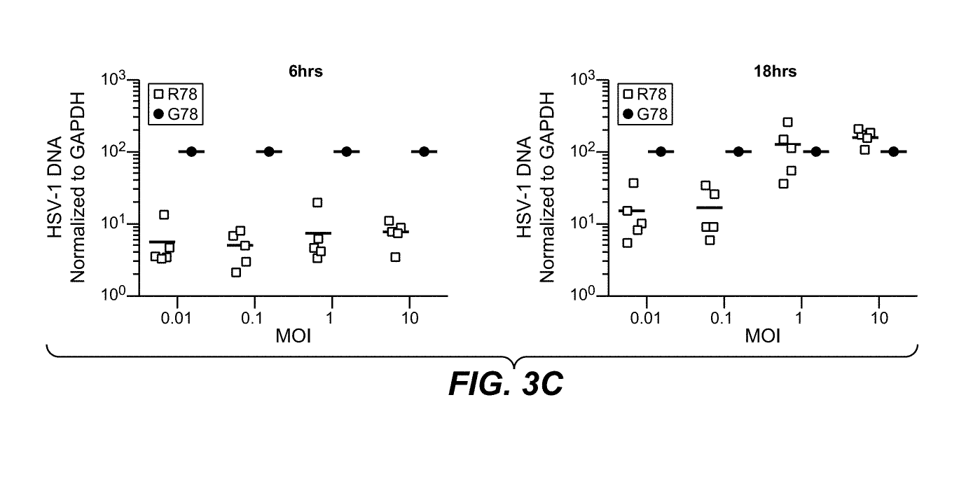

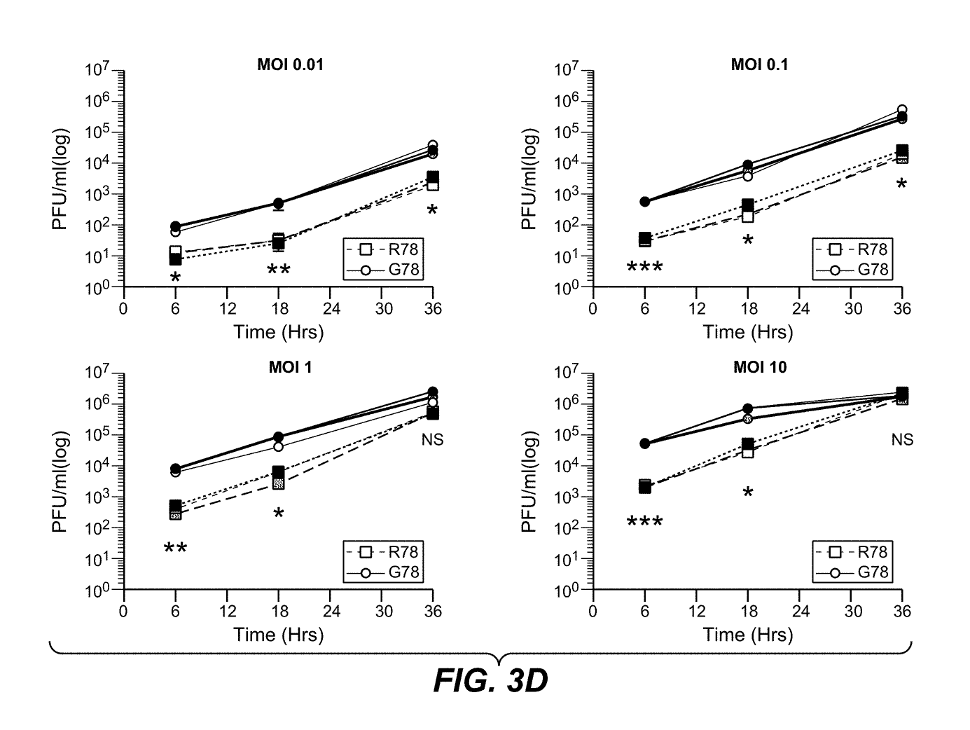

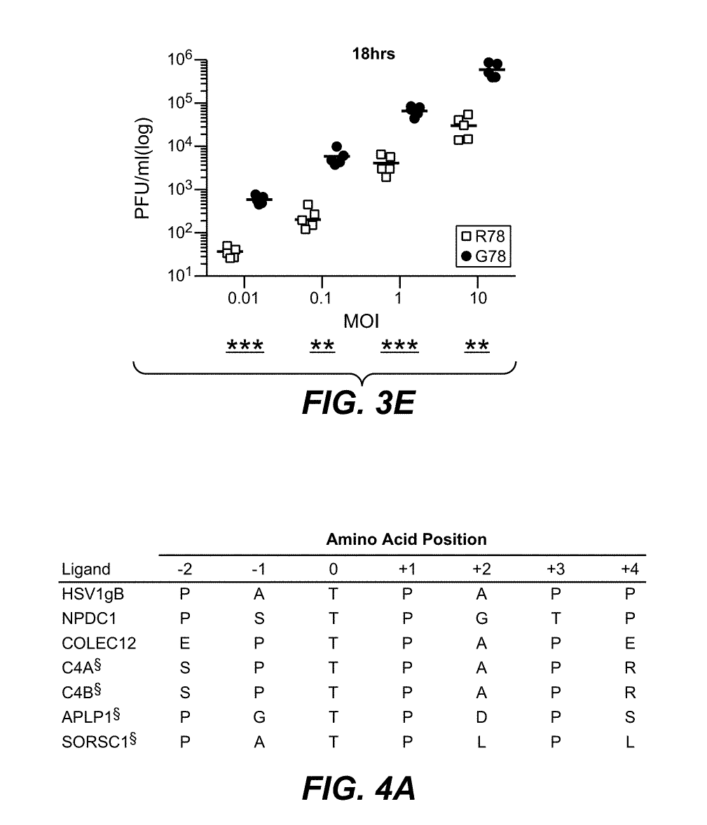

Since PILRA is an entry receptor for HSV-1 virus and reduced interaction between the R78 variant of PILRA and HSV-1 gB was demonstrated herein, the biological impact of the R78 variant on HSV-1 infection was to be assessed. Human monocyte-derived macrophages (hMDMs) were isolated and differentiated from five pairs of healthy volunteers homozygous for G78 or R78 variant PILRA (matched for age, gender and ethnicity). The hMDMs were infected with HSV-1 at different multiplicities of infection (MOI) (0.01, 0.1, 1 and 10). Infectivity at various times post infection was observed by virus-induced cytopathic effect (CPE) and measured using the LDH cytotoxicity assay. Extensive CPE was detected in G78 variant of PILRA expressing hMDMs at 18 hrs post infection, including loss of cell shape, cell rounding, increased volume, birefringence, formation of lumps, and multinucleated giant cells (syncytia) (FIG. 3A). hMDMs from R78 PILRA donors also showed significantly less HSV-1-induced cytotoxicity at 18 hrs post infection in the LDH assay at 0.01, 0.1, or 1 MOI (FIG. 3B). In comparison, CPE was noticeably less pronounced in hMDMs expressing the R78 (AD-protective) PILRA variant, even at 1 MOI=1. hMDMs from R78 PILRA donors also showed significantly less HSV-1-induced cytotoxicity at 18 hrs post infection in the LDH assay at 0.01, 0.1, or 1 MOI (FIG. 3B). The difference was no longer significant at 10 MOI or if the infection was allowed to proceed for 36 hrs, except at the lowest MOI of 0.01. These data suggested that hMDMs from R78 PILRA donors exhibit lower rates of HSV-1 infection, but this reduced susceptibility could be overcome by increased magnitude or duration of HSV-1 exposure—consistent with reduced, but not eliminated, association between HSV-1 gB and the R78 variant of PILRA.

To determine if the HSV-1-induced cytopathic effect correlated with viral replication, DNA from HSV-1-infected hMDMs was extracted and quantitated HSV-1 DNA by qPCR, compared to human GAPDH to normalize for cell numbers. hMDMs from R78 donors showed 5-10 fold decreased amount of HSV-1 DNA at 6 hrs with all doses (0.01, 0.1, 1 and 10 MOI) and at 18 hrs with lower doses (0.01 and 0.1 MOI) of virus, compared to their G78 counterparts (FIG. 3C). We also measured the amount of infectious HSV-1 production by harvesting supernatants from HSV-1-infected hMDMs and measuring viral titer by plaque assay on Vero cells. Viral PFU correlated very well with the levels of viral replication measured by qPCR (FIG. 3D, E). Taken together, these data suggest that macrophages expressing the R78 variant of PILRA (AD-protective) are less susceptible to HSV-1 infection than those expressing the G78 variant of PILRA (AD-risk), which could be explained by decreased viral entry due to reduced affinity of the R78 variant of PILRA toward HSV-1 gB.

### Example 7

### PILRA Ligands

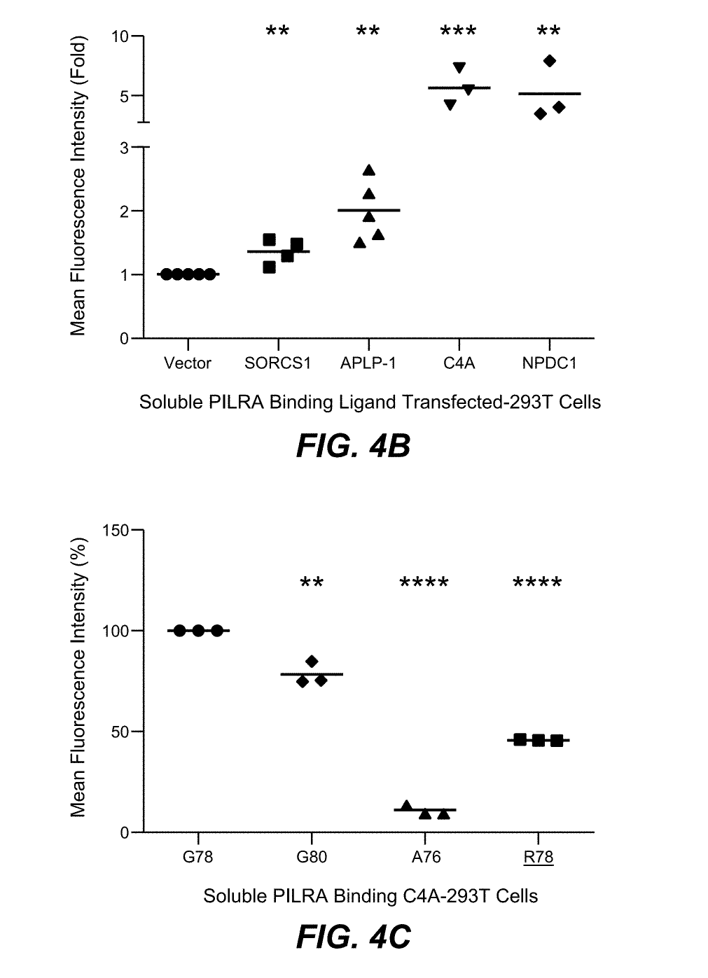

The function of PILRA and PILRB in the immune system is not understood in detail; however, PILRA has been reported to dampen the innate immune response by negatively regulating NK cell activation and neutrophil and monocyte infiltration. PILRA deficiency can lead to dysregulation of inflammatory processes in affected tissues, resulting in increased inflammatory cytokine production and severity of mouse arthritis. While the relevant ligand(s) for PILRA activation in these settings is unclear, a peptide motif for PILRA interaction has been established previously (FIG. 4A) that includes an O-glycosylated threonine, an invariant proline at the +1 position, and additional prolines at the −1 or −2 and +3 or +4 positions (see e.g. Sun et al., J. Biol. Chem. 287, 15837-50 (2012), and Kuroki et al., Proc. Natl. Acad. Sci. U.S.A. 111, 8877-82 (2014)). Of note, PILRA is capable of binding murine CD99 and human NPCD1 (both contain the consensus motif), but not human CD99 or murine NPCD1 (both lack the consensus motif), suggesting divergence between human and mouse in the range of endogenous ligands bound by PILRA. Unknown endogenous PILRA ligands were sought to be identified by searching for human proteins containing the PTPXP, PTPXXP, PXTPXP or PXTPXXP motif. Proteins with the motif that have previously been shown to be O-glycosylated in human cerebral spinal fluid were considered (see e.g., Halim et al., J. Proteome Res. 12, 573-84 (2013)), and the binding of these proteins to PILRA variants was measured. Using FACS assay, complement component 4A (C4A) was found to bind to the G78 variant of PILRA in a manner comparable to NPDC1, while APLP1 and SORCS1 showed less PILRA interaction (FIG. 4B). Furthermore, it was demonstrated that the R78 variant of PILRA has reduced binding for C4A (FIG. 4C).

### Example 8

Anti-mPILRA Antibody Can Block Mouse CD99 and C12orf53 Binding to mPILRA in PILRA ECD-Based Competitive ELISA and 293-PILRA Cell Based Competitive ELISA

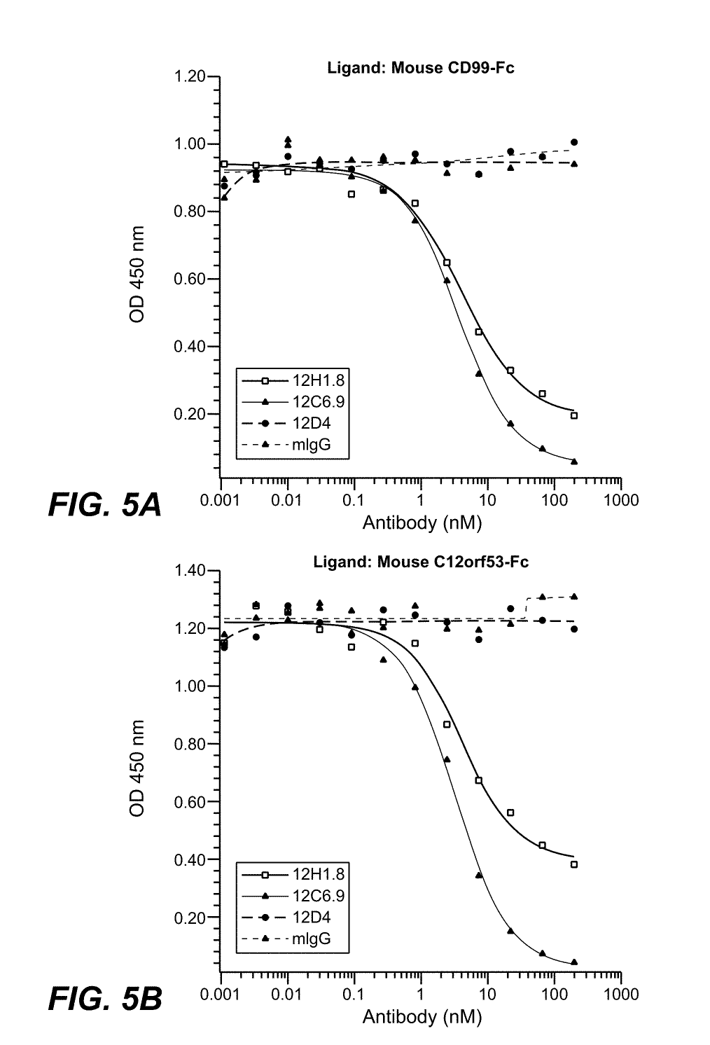

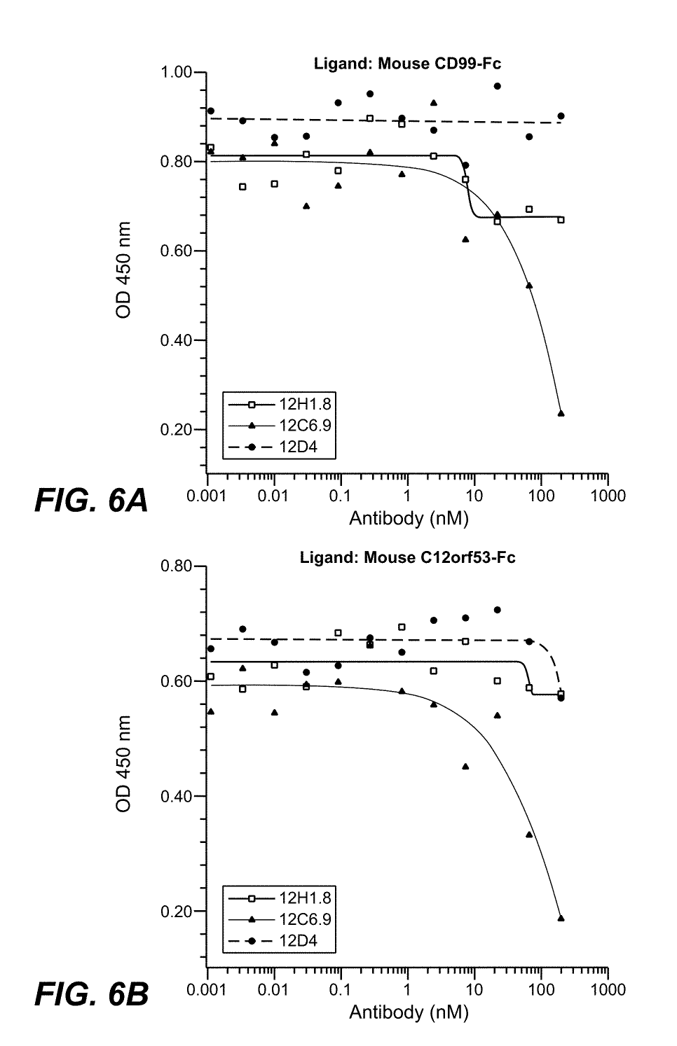

The anti-mouse PILRA 12H1.8 and 12C6.9 antibodies were evaluated for their activities in blocking binding of PILRA ligands. In the PILRA ECD-based competitive ELISA, antibody 12H1.8 partially blocked binding of mouse CD99-Fc (FIG. 5A) and C12orf53-Fc (FIG. 5B) to mouse PILRA ECD. Antibody 12C6.9 blocked binding of both ligands to mouse PILRA ECD. Mouse IgG (mIgG) and isotype control antibody 12D4 that did not bind to PILRA did not show any blocking activity as expected. In the 293-PILRA cell-based competitive ELISA, antibody 12H1.8 showed partial blocking activity for mouse CD99-Fc binding (FIG. 6A) and no obvious blocking activity for mouse C12orf53-Fc binding (FIG. 6B) to cell surface PILRA. 12C6.9 blocked binding of both ligands to cell surface PILRA. From the dose dependent curves, 12C6.9 blocked binding of both ligands to recombinant PILRA ECD more efficiently than to cell surface PILRA.

### Example 9

### Murine PILRA Ligand Blocking Using SPR Results

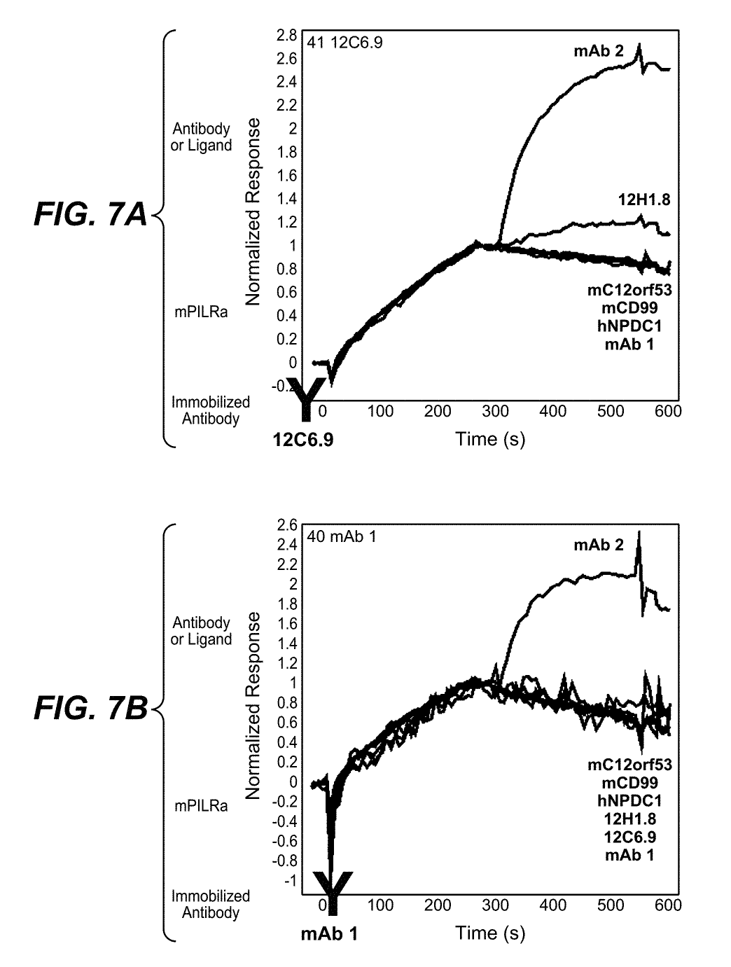

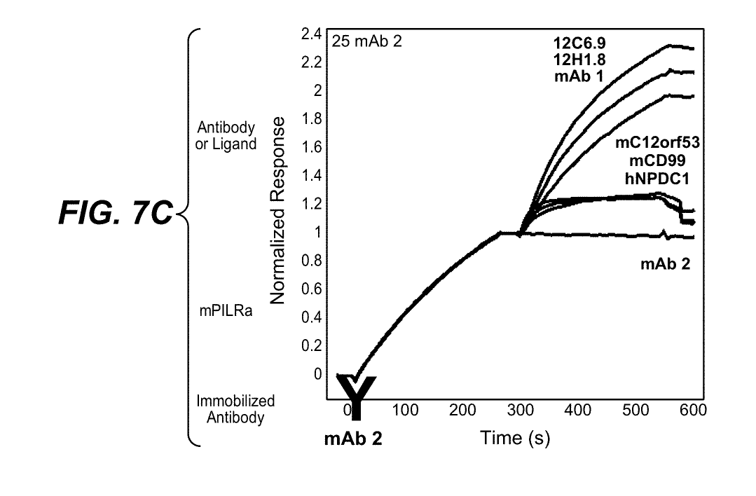

Anti-murine PILRA antibodies directly immobilized via amine coupling were binned against each other and murine PILRA ligands to identify ligand blocking clones of interest. In-house Fc tagged ligands, murine CD99, murine C12orf53 (mPIANP) and human NPDC1 were used in the experiment. When 12C6.9 is immobilized on the chip (surface) and allowed to bind PILRA, all three ligands are unable to bind and only the non-blocking positive control mAb 2 is able to bind well (FIG. 7A). Additionally, when 12C6.9 is in solution it is able to bind PILRA in a similar manner as blocking clone positive control mAb 1. When the blocking antibody mAb 1 is immobilized, all three ligands as well as 12C6.9, 12H1.8 and mAb 1 are unable to bind and only the non-blocking positive control mAb 2 is able to bind well (FIG. 7B). When the non-blocking positive control mAb 2 is immobilized, all three ligands as well as 12C6.9, 12H1.8 and mAb 1 bind (FIG. 7C). Immobilization of 12H1.8 disrupted its binding to PILRA, but solution data demonstrates it behaves similarly to mAb 1 and 12C6.9 in solution, binding in the presence of non-blocking clone mAb 2 and not binding in the presence of blocking clone mAb 1. This data demonstrates that by SPR and using recombinant proteins clones 12C6.9 and 12H1.8 bind to murine PILRA in a manner that competes with ligand binding.

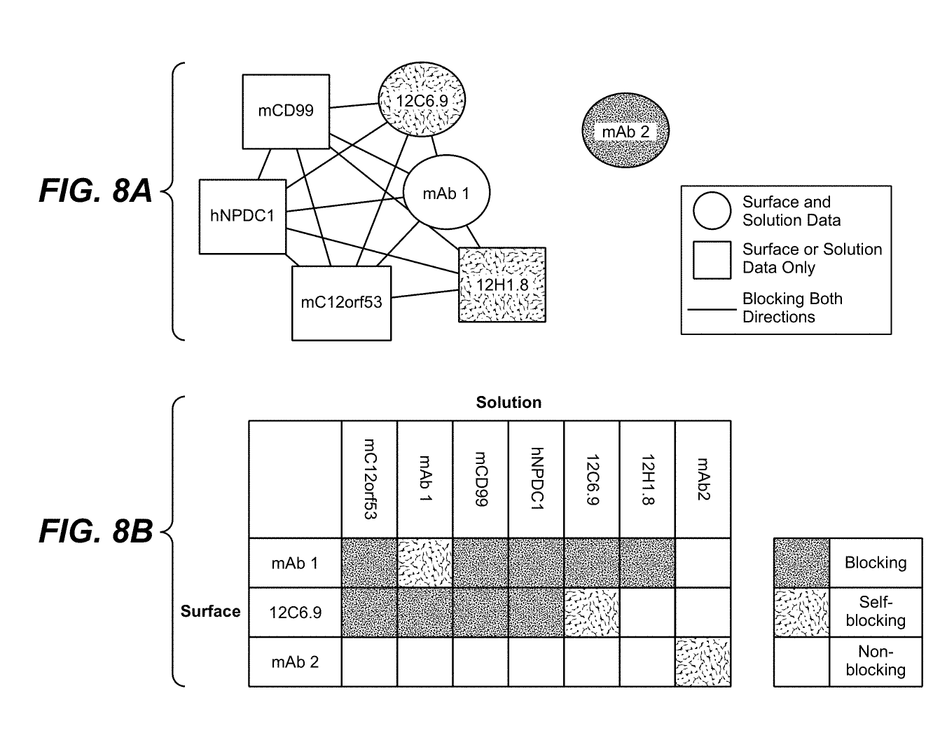

Antibody and ligand bins based on SPR binning data are shown in a network plot by chords connecting antibodies and ligands with overlapping epitopes (FIG. 8A). Antibodies 12C6.9 and 12H1.8 demonstrate similar binding to murine PILRA as the ligands tested, mCD99, mC12orf53, and hNPDC1 and blocking clone mAb 1. Direct blocking and non-blocking interactions are shown in the form of a heatmap (FIG. 8B).

Table of Sequences

| | | SEQ |
|---|---|---|
| NAME | SEQUENCE | ID NO |
| Human PILRA | MGRPLLLPLLPLLLPPAFLQPSGSTGSGPSYLYGVTQPKHLSASMGGSVEIPFSEY | 1 |
| G78 variant | 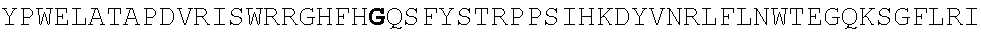 | |
| (incl. signal | SNLQKQDQSVYFCRVELDTRSSGRQQWQSIEGTKLSITQAVTTTTQRPSSMTTTWR | |
| peptide | LSSTTTTTGLRVTQGKRRSDSWHISLETAVGVAVAVTVLGIMILGLICLLRWRRRK | |
| (underlined)) | GQQRTKATTPAREPFQNTEEPYENIRNEGQNTDPKLNPKDDGIVYASLALSSSTSP | |
| | RAPPSHRPLKSPQNETLYSVLKA | |
| Human PILRA, | MGRPLLLPLLPLLLPPAFLQPSGSTGSGPSYLYGVTQPKHLSASMGGSVEIPFSFY | 2 |
| R78 variant | 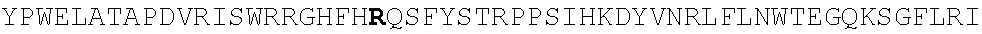 | |
| Q9UKJ1 | SNLQKQDQSVYFCRVELDTRSSGRQQWQSIEGTKLSITQAVTTTTQRPSSMTTTWR | |
| (incl. signal | LSSTTTTTGLRVTQGKRRSDSWHISLETAVGVAVAVTVLGIMILGLICLLRWRRRK | |
| peptide | GQQRTKATTPAREPFQNTEEPYENIRNEGQNTDPKLNPKDDGIVYASLALSSSTSP | |
| (underlined)) | RAPPSHRPLKSPQNETLYSVLKA | |
| Human PILRA | MGRPLLLPLLPLLLPPAFLQPSGSTGSGPSYLYGVTQPKHLSASMGGSVEIPFSFY | 3 |
| L279 variant | YPWELATAPDVRISWRRGHFHGQSFYSTRPPSIHKDYVNLRLFLNWTEGQKSGFLRI | |
| (incl. signal | SNLQKQDQSVYFCRVELDTRSSGRQQWQSIEGTKLSITQAVTTTTQRPSSMTTTWR | |
| peptide | LSSTTTTTGLRVTQGKRRSDSWHISLETAVGVAVAVTVLGIMILGLICLLRWRRRK | |
| (underlined)) | 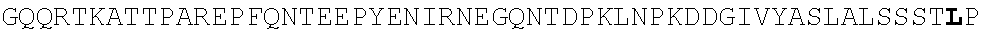 | |
| | RAPPSHRPLKSPQNETLYSVLKA | |
| Human | MGPASPAARGLSRRPGQPPLPLLLPLLLLLLRAQPAIGSLAGGSPGAAEAPGSAQV | 4 |
| Amyloid-like | AGLCGRLTLHRDLRTGRWEPDPQRSRRCLRDPQRVLEYCRQMYPELQIARVEQATQ | |
| protein 1 | AIPMERWCGGSRSGSCAHPHHQVVPFRCLPGEFVSEALLVPEGCRFLHQERMDQCE | |
| (APLP1) | SSTRRHQEAQEACSSQGLILHGLGMLLPCGSDRFRGVEYVCCPPPGTPDPSGTAVG | |
| P51693 | DPSTRSWPPGSRVEGAEDEEEEESFPQPVDDYFVEPPQAEEEEETVPPPSSHTLAV | |
| (incl. signal | VGKVTPTPRPTDGVDIYFGMPGEISEHEGFLRAKMDLEERRMRQINEVMREWAMAD | |
| peptide | NQSKNLPKADRQALNEHFQSILQTLEEQVSGERQRLVETHATRVIALINDQRRAAL | |
| (underlined)) | EGFLAALQADPPQAERVLLALRRYLRAEQKEQRHTLRHYQHVAAVDPEKAQQMRFQ | |
| | VHTHLQVIEERVNQSLGLLDQNPHLAQELRPQIQELLHSEHLGPSELEAPAPGGSS | |
| | EDKGGLQPPDSKDDTPMTLPKGSTEQDAASPEKEKMNPLEQYERKVNASVPRGFPFP | |
| | HSSEIQRDELAPAGTGVSREAVSGLLIMGAGGGSLIVLSMLLLRRKKPYGAISHGV | |
| | VEVDPMLTLEEQQLRELQRHGYENPTYRFLEERP | |
| Human | MPLTPEPPSGRVEGPPAWEAAPWPSLPCGPCIPIMLVLATLAALFILTTAVLAERL | 5 |
| Transmembrane | FRRALRPDPSHRAPTLVWRPGGELWIEPMGTARERSEDWYGSAVPLLTDRAPEPPT | |
| Protein | QVGTLEARATAPPAPSAPNSAPSNLGPQTVLEVPARSTFWGPQPWEGRPPATGLVS | |
| C16orf54 | WAEPEQRPEASVQFGSPQARRQRPGSPDPEWGLQPRVTLEQISAFWKREGRTSVGF | |
| Q6UWD8 | | |
| Human | MRLLWGLIWASSFFTLSLQKPRLLLFSPSVVHLGVPLSVGVQLQDVPRGQVVKGSV | 6 |
| Complement | FLRNPSRNNVPCSPKVDFTLSSERDFALLSLQVPLKDAKSCGLHQLLRGPEVQLVA | |
| C4A | HSPWLKDSLSRTTNIQGINLLFSSRRGHLFLQTDQPIYNPGQRVRYRVFALDQKMR | |
| P0C0L4 | PSTDTITVMVENSHGLRVRKKEVYMPSSIFQDDFVIPDISEPGTWKISARFSDGLE | |
| (incl. signal | SNSSTQFEVKKYVLPNFEVKITPGKPYILTVPGHLDEMQLDIQARYIYGKPVQGVA | |
| peptide | YVRFGLLDEDGKKTFFRGLESQTKLVNGQSHISLSKAEFQDALEKLNMGITDLQGL | |
| (underlined)) | RLYVAAAIIESPGGEMEEAELTSWYFVSSPFSLDLSKTKRHLVPGAPFFLLQALVRE | |
| | MSGSPASGIPVKVSATVSSPGSVPEVQDIQQNTDGSGQVSIPIIIPQTISELQLSV | |
| | SAGSPHPAIARLTVAAPPSGGPGFSLSIERPDSRPPRVGDTLNLNLRAVGSGATFSH | |
| | YYYMILSRGQIVFMNREPKRTLTSVSVFVDHHLAPSFYFVAFYYHGDHPVANSLRV | |
| | DVQAGACEGKLELSVDGAKQYRNGESVKLHLETDSLALVALGALDTALYAAGSKSH | |
| | KPLNMGKVFEAMNSYDLGCGPGGGDSALQVFQAAGLAFSDGDQWTLSRKRLSCPKE | |
| | KTTRKKRNVNFQKAINEKLGQYASPTAKRCCQDGVTRLPMMRSCEQRAARVQQPDC | |
| | REPFLSCCQFAESLRKKSRDKGQAGLQRALEILQEEDLIDEDDIPVRSFFPENWLW | |
| | RVETVDRFQILTLWLPDSLTTWEIHGLSLSKTKGLCVATPVQLRVFREFHLHLRLP | |
| | MSVRRFEQLELRPVLYNYLDKNLTVSVHVSPVEGLCLAGGGGLAQQVLVPAGSARP | |
| | VAFSVVPTAAAAVSLKVVARGSFEFPVGDAVSKVLQIEKEGAIHREELVYELNPLD | |
| | HRGRTLEIPGNSDPNMIPDGDFNSYVRVTASDPLDTLGSEGALSPGGVASLLRLPR | |
| | GCGEQTMIYLAPTLAASRYLDKTEQWSTLPPETKDHAVDLIQKGYMRIQQFRKADG | |
| | SYAAWLSRDSSTWLTAFVLKVLSLAQEQVGGSPEKLQETSNWLLSQQQADGSFQDP | |
| | CPVLDRSMQGGLVGNDETVALTAFVTIALHHGLAVFQDEGAEPLKQRVEASISKAN | |
| | SFLGEKASAGLLGAHAAAITAYALTLTKAPVDLLGVAHNNLMAMAQETGDNLYWGS | |
| | VTGSQSNAVSPTPAPRNPSDPMPQAPALWIETTAYALLHLLLHEGKAEMADQASAW | |
| | LTRQGSFQGGFRSTQDTVIALDALSAYWIASHTTEERGLNVTLSSTGRNRFKSHAL | |
| | QLNNRQIRGLEELQFSLGSKINVKVGGNSKGTLKVLRTYNVLDMKNTTCQDLQIE | |
| | VTVKGHVEYTMEANEDYEDYEYDELPAKDDPDAPLQPVTPLQLFEGRRNRRRREAP | |
| | KVVEEQESRVHYTVCIWRNGKVGLSGMAIADVTLLSGFHALRADLEKLTSLSDRYV | |
| | SHFETEGPHVLLYFDSVPTSRECVGFEAVQEVPVGLVQPASATLYDYYNPERRCSV | |
| | FYGAPSKSRLLATLCSAEVCQCAEGKCPRQRRALERGLQDEDGYRMKFACYYPRVE | |
| | YGFQVKLREDSRAAFRLFETKITQVLHFTKDVKAAANQMRNFLVRASCRLRLEPG | |
| | KEYLIMGLDGATYDLEGHPQYLLDSNSWIEEMPSERLCRSTRQRAACAQLNDFLQE | |
| | YGTQGCQV | |
| Huamn | MRLLWGLIWASSFFTLSLQKPRLLLFSPSVVHLGVPLSVGVQLQDVPRGQVVKGSV | 7 |
| Complement | FLRNPSRNNVPCSPKVDFTLSSERDFALLSLQVPLKDAKSCGLHQLLRGPEVQLVA | |
| C4B | HSPWLKDSLSRTTNIQGINLLFSSRRGHLFLQTDQPIYNPGQRVRYRVFALDQKMR | |
| P0C0L5 | PSTDTITVMVENSHGLRVRKKEVYMPSSIFQDDFVIPDISEPGTWKISARFSDGLE | |
| (incl. signal | SNSSTQFEVKKYVLPNFEVKITPGKPYILTVPGHLDEMQLDIQARYTIYGKPVQGVA | |
| peptide | YVRFGLLDEDGKKTFFRGLESQTKLVNGQSHISLSKAEFQDALEKLNMGITDLQGL | |
| (underlined)) | RLYVAAAAIIESPGGEMEEAELTSWYFVSSPFSLDLSKTKRHLVPGAPFLLQALVRE | |
| | MSGSPASGIPVKVSATVSSPGSVPEVQDIQQNTDGSGQVSIPIIIPQTISELQLSV | |
| | SAGSPHPAIARLTVAAPPSGGPGFLSIERPDSRPPRVGDTLNLNLRAVGSGATFSH | |
| | YYYMILSRGQIVFMNREPKRTLTSVSVFVDHHLAPSFYFVAFYYHGDHPVANSLRV | |
| | DVQAGACEGKLELSVDGAKQYRNGESVKLHLETDSLALVALGALDTALYAAGSKSH | |
| | KPLNMGKVFEAMNSYDLGCGPGGGDSALQVFQAAGLAFSDGDQWTLSRKRLSCPKE | |
| | KTTRKKRNVNFQKAINEKLGQYASPTAKRCCQDVTRLPMMRSCEQRAARVQQPDC | |
| | REPFLSCCQFAESLRKKSRDKGQAGLQRALEILQEEDLIDEDDIFVRSFPFPENWLW | |
| | RVETVDRFQILTLWLPDSLTTWEIHGLSLSKTKGLCVATPVQLRVFREFHLHLRLP | |
| | MSVRRFEQLELRPVLYNYLDKNLTVSVHVSPVEGLGLAGGGGLAQQVLVPAGSARP | |
| | VAFSVVPTAATAVSLKVVARGSFEFPVGDAVSKVLQIEKEGAIHREELVYELNPLD | |
| | HRGRTLEIPGNSDPNMIPDGDFNSYVRVTASDPLDTLGSEGALSPGGVASLLRLPR | |
| | GCGEQTMIYLAPTLAASRYLDKTEQWSTLPPETKDHAVDLIQKGYMRIQQFRKADG | |
| | SYAAWLSRGSSTWLTAFVLKVLSLAQEQVGGSPEKLQETSNWLLSQQQADGSFQDL | |
| | SPVIHRSMQGGLVGNDETVALTAFVTIALHHGLAVFQDEGAEPLKQRVEASISKAS | |
| | SFLGEKASAGLLGAHAAAITAYALTLTKAPADLRGVAHNNLMAMAQETGDNLYWGS | |
| | VTGSQSNAVSPTPAPRNPSDPMPQAPALWIETTAYALLHLLLHEGKAEMADQAAAW | |
| | LTRQGSFQGGFRSTQDTVIALDALSAYWIASHTTEERGLNVTLSSTGRNGFKSHAL | |
| | QLNNRQIRGLEEELQFSLGSKINVKVGGNSKGTLKVLRTYNVLDMKNTTCQDLQIE | |
| | VTVKGHVEYTMEANEDYEDYEYDELPAKDDPDAPLQPVTPLQLFEGRRNRRRREAP | |
| | KVVEEQESRVHYTVCIWRNGKVGLSGMAIADVTLLSGFHALRADLEKLTSLSDRYV | |
| | SHFETEGPHVLLYFDSVPTSRECVGFEAVQEVPVGLVQPASATLYDYYNPERRCSV | |
| | FYGAPSKSRLLATLCSAEVCQCAEGKCPRQRRALERGLQDEDGYRMKFACYYPRVE | |
| | YGFQVKVLREDSRAAFRLFETKITQVLHFTKDVKAAANQMRNFLVRASCRLRLEPG | |
| | KEYLIMGLDGATYDLEGHPQYLLDSNSWIEEMPSERLCRSTRQRAACAQLNDFLQE | |
| | YGTQGCQV | |
| Human C-type | MDTTRYSKWGGSSEEVPGGWGRWVHWSRRPLFLALAVLVTTVLWAVILSILLSKA | 8 |
| lectin domain | STERAALLDGHDLLRTNASKQTAALGALKEEVGDCHSCCSGTQAQLQTTRAELGEA | |
| family 4 | QAKLMEQESALRELRERVTQGLAEAGRGREDVRTELFRALEAVRLQNNSCEPCPTS | |
| member G | WLSFEGSCYFFSVPKTTWAAAQDHCACASAHLVIVGGLDEQGFLTRNTRGRGYWLG | |
| (CLEC4G) | LRAVRHLGKVQGYQWVDGVSLSFSHWNQGEPNDAWGRENCVMMLHTGLWNDAPCDS | |
| Q6UXB4 | EKDGWICEKRHNC | |
| Human | MKDDFAEEEEVQSFGYKRFGIQEGTQCTKCKNNWALKFSIILLYILCALLTITVAI | 9 |
| Collectin-12 | LGYKVVEKMDNVTGGMETSRQTYDDKLTAVESDLKKLGDQTGKKAISTNSELSTFR | |
| (COLEC12) | SDILDLRQQLREITEKTSKNKDTLEKLQASGDALVDRQSQLKETLENNSFLITTVN | |
| Q5KU26 | KTLQAYNGYVTNLQQDTSVLQGNLQNQMYSHNVVIMLNNLNLTQVQQRNLITNLQ | |
| | RSVDDTSQAIQRIKNDFQNLQQVFLQAKKDTDWLKEKVQSLSTLAANNSALAKANN | |
| | DTLEDMNSQLNSFTGQMENITTISQANEQNLKDLQDLHKDAENRTAIKFNQLEERF | |
| | QLFETDIVNIISNISYTAHHLRTLTSNLNEVRTTCTDTLTKHTDDLTSLNNTLANI | |
| | RLDSVSLRMQQDLMRSRLDTEVANLSVIMEEMKLVDSKHGQLIKNFTILQGPPGPR | |
| | GRPGDRGSQGPPGPTGNKGQKGEKGEPGPPGPAGERGPIGPAGPPGERGGKGSKGS | |
| | QGPKGSRGSPGKPGPQGSSGDPGPPGPPGKEGLPGPQGPPGFQGLQGTVGEPGVPG | |
| | PRGLPGLPGVPGMPGPKGPPGPPGPSGAVVPLALQNEPTPAPEDNGCPPHWKNFTD | |
| | KCYYFSVEKEIFEDAKLFCEDKSSHLVFINTREEQQWIKKQMVGRESHWIGLTDSE | |
| | RENEWKWLDGTSPDYKNWKAGQPDNWGHGHGPGEDCAGLIYAGQWNDFQCEDVNNF | |
| | ICEKDRETVLSSAL | |
| Human | MRMSVGLSLLLPLSGRTFLLLLSVVMAQSHWPSEPSEAVRDWENQLEASMHSVLSD | 10 |
| Dystroglycan | LHEAVPTVVGIPDGTAVVGRSFRVTIPTDLIASSGDIIKVSAAGKEALPSWLHWDS | |
| (DAG1) | QSHTLEGLPLDTDKGVHYISVSATRLGANGSHIPQTSSVFSIEVYPEDHSELQSVR | |
| Q14118 | TASPDPGEVVSSACAADEPVTVLTVILDADLTKMTPKQRIDLLHRMRSFSEVELHN | |
| (incl. signal | MKLVPVVNNRLFDMSAFMAGPGNAKKVVENGALLSWKLGCSLNQNSVPDIHGVEAP | |
| peptide | AREGAMSAQLGYPVVGWHIANKKPPLPKRVRRQIHATPTPVTAIGPPTTAIQEPPS | |
| (underlined)) | RIVPTPTSPAIAPPTETMAPPVRDPVPGKPTVTIRTRGAIIQTPTLGPIQPTRVSE | |
| | AGTTVPGQIRPTMTIPGYVEPTAVATPPTTTTKKPRVSTPKPATPSTDSTTTTTRR | |
| | PTKKRRTPRPVPRVTTKVSITRLETASPPTKRIRTTSGVPRGGEPNQRPELKNHID | |
| | RVDAWVGTYFEVKIPSDTFYDHEDTTTDKLKLTLKLREQQLVGEKSWVQFNSNSQL | |
| | MYGLPDSSHVGKHEYFMHATDKGGLSAVDAFEIHVHRRPQGDRAPARFKAKFVGDP | |
| | ALVILNDIHKKIALVKKLAFAFGDRNCSTITLQNITRGSIVVEWTNNTLPLEPCIPKE | |
| | QIAGLSRRIAEDDGKPRPAFSNALEPDFKATSITVTGSGSCRHLQFIPVVPPRRVP | |
| | SEAPPTEVPDRDPEKSSEDDVYLHTVIPAVVVAAILLIAGIIAMICYRKKRKGKLT | |
| | LEDQATFIKKGVPIIFADELDDSKPPPSSSMPLILQEEKAPLPPPEYPNQSVPETT | |
| | PLNQDTMGEYTPLRDEDPNAPPYQPPPPFTAPMEGKGSRPKNMTPYRSPPPYVPP | |
| Human Protein | MLLPGRARQPPTPQPVQHPGLRRQVEPPGQLLRLFYCTVLVCSKEISALTDFSGYL | 11 |
| eba-1 homolog | TKLLQNHTTYACDGDYLNLQCPRHSTISVQSAFYGQDYQMCSSQKPASQREDSLTC | |
| C | VAATTFQKVLDECQNQRACHLLVNSRVFGPDLCPGSSKYLLVSFKCQPNEILKNKTV | |
| (EVA1C) | CEDQELKLHCHESKFLNIYSATYGRRTQERDICSSKAERLPPFDCLSYSALQVLSR | |
| P58658 | RCYGKQRCKIIVNNHHHFGSPCLPGVKKYLTVTYACVPKNILTAIDPAIANLKPSLK | |
| (incl. signal | QKDGEYGINFDPSGSKVLRKDGILVSNSLAAFAYIRAHPERAALLFVSSVCIGLAL | |
| peptide | TLCALVIRESCAKDFRDLQLGREQLVPGSDKVEEDSEDEEEEEDPSESDFPGELSG | |
| (underline)) | FCRTSYPIYSSIEAAELAERIERREQIIQEIWMNSGLDTSLPRNMGQFY | |
| Human Low | MEEGQYSEIEELPRRRCCRRGTQIVLLGLVTAALWAGLLTLLLLWHWDTTQSLKQL | 12 |
| affinity | EERAARNVSQVSKNLESHHGDQMAQKSQSTQISQELEELRAEQQRLKSQDLELSWN | |
| immunoglobulin | LANGLQADLSSFKSQELNERNEASDLLERLREEVTKLRMELQVSSGFVCNTCPEKWY | |
| epsilon Fc | NFQRKCYYFGKGTKQWVHARYACDDMEGQLVSIHSPEEQDFLTKHASHTGSWIGLR | |
| receptor | NLDLKGEFIWVDGSHVDYSNWAPGEPTSRSGEDCVMMRGSGRWNDAFCDRKLGAW | |
| (FceRII) | VCDRLATCTPPASEGSAESMGPDSRPDPDGRLPTPSAPLHS | |
| P06734 | | |
| HSV-1 | MHQGAPSWGRRWFVVWALLGLTLGVLVASAAPTSPGTPGVAAATQAANGGPATPAP | 13 |
| glycoprotein | PPLGAAPTGDPKPKKNKKPKNPTPPRPAGDNATVAAGHATLREHLRDIKAENTDAN | |
| B | FYVCPPPTGATVVQFEQPRRCPTRPEGQNYTEGIAVVFKENIAPYKFKATMYYKDV | |
| (gB) | TVSQVWFGHRYSQFMGIFEDRAPVPFEEVIDKINAKGVCRSTAKYVRNNLETTAFH | |
| P06437 | RDDHETDMELKPANAATRTSRGWHTTDLKYNPSRVEAFHRYGTTVNCIVEEVDARS | |
| (incl. signal | VYPYDEFVLATGDFVYMSPFYGYREGSHTEHTTYAADRFKQVDGFYARDLTTKARA | |
| peptide | TAPTTRNLLTTPKFTVAWDWVPKRPSVCTMTKWQEVDEMLRSEYGGSFRFSSDAIS | |
| (underlined)) | TTFTTNLTEYPLSRVDLGDCIGKDARDAMDRIFARRYNATHIKVGQPQYYQANGGF | |
| | LIAYQPLLSNTLAELYVREHLREQSRKPPNPTPPPPGASANASVERIKTTSSIEFA | |
| | RLQFTYNHIQRHVNDMLGRVAIAWCELQNHELTLWNEARKLNPNAIASVTVGRRVS | |
| | ARMLGDVMAVSTCVPVAADNVIVQNSMRISSRPGACYSRPLVSFRYEDQGPLVEGQ | |
| | LGENNELRLTRDAIEPCTVGHRRYFTFGGGYVYFEEYAYSHQLSRADITTVSTFID | |
| | LNITMLEDHEFVPLEVYTRHEIKDSGLLDYTEVQRRNQLHDLRFADIDTVIHADAN | |
| | AAMFAGLGAFFEGMGDLGRAVGKVVMGIVGGVVSAVSGVSSFMSNPFGALAVGLLV | |
| | LAGLAAAFFAFRYVMRLQSNPMKALYPLTTKELKNPTNPDASGEGEEGGDFDEAKL | |
| | AEAREMIRYMALVSAMERTEHKAKKKGTSALLSAKVTDMVMRKRRNTNYTQVPNKD | |
| | GDADEDDL | |
| Human | MGAARSPPSAVPGPLLGLLLLLGVLAPGGASLRLLDHRALVCSQPGLNCTVKNST | 14 |
| Interleukin- | CLDDSWIHPRNLTPSSPKDLQIQLHFAHTQQGDLFPVAHIEWTLQTDASILYLEGA | |
| 17 receptor A | ELSVLQLNTNERLCVRFEFLSKLRHHHRRWRFTFSHFVVDPDQEYEVTVHHLPKPI | |
| (IL17RA) | PDGDPNHQSKNFLVPDCEHARMKVTTPCMSSGSLWDPNITVETLEAHQLRVSFTLW | |
| Q96F46 | NESTHYQILLTSFPHMENHSCFEHMHHIPAPRPEEFHQRSNVTLTRNLKGCCRHQ | |
| (incl signal) | VQIQPFFSSCLNDCLRHSATVSCPEMPDTPEPIPDYMPLWVYWFITGISILLVGSV | |
| peptide | ILLIVCMTWRLAGPGSEKYSDDTKYTDGLPAADLIPPPLKPRKVWIIYSADHPLYV | |
| (underlined)) | DVVLKFAQFLLTACGTEVALDLLEEQAISEAGVMTWVGRQKQEMVESNSKIIVLCS | |
| | RGTRAKWQALLGRGAPVRLRCDHGKPVGDLFTAAMNMILPDFKRPACFGTYVVCYF | |
| | SEVSCDGVPDLFGAAPRYPLMDRFEEVYFRIQDLEMFQPGRMHRVGELSGDNYLR | |
| | SPGGRQLRAALDRFRDWQVRCPDWFECENLYSADDQDAPSLDEEVFEEPLLPPGTG | |
| | IVKRAPLVREPGSQACLAIDPLVGEEGGAAVAKLEPHLQPRGQPAPQPLHTLVLAA | |
| | EEGALVAAVEPGPLADGAAVRLALAGEGEACPLLGSPGAGRNSVLFLPVDPEDSPL | |
| | GSSTPMASPDLLPEDVREHLEGLMLSLFEQSLSCQAQGGCSRPAMVLTDPHTYEE | |
| | EQRQSVQSDQGYISRSSPQPPEGLTEMEEEEEEEQDPGKPALPLSPEDLESLRSLQ | |
| | RQLLFRQLQKNSGWDTMGSESEGPSA | |
| Human | MTLTLSVLICLGLSVGPRTCVQAGTLPKPTLWAEPASVIARGKPVTLWCQGPLETE | 15 |
| Leukocyte | EYRLDKEGLPWARKRQNPLEPGAKAKFHIPSTVYDSAGRYRCYYETPAGWSEPSDP | |
| immunoglubulne- | LELVATGFYAEPTLLALPSPVVASGGNVTLQCDTLDGLLTFVLVEEEQKLPRTLYS | |
| like | QKLPKGPSQALFPVGPVTPSCRWRFRCYYYYRKNPQVWSNPSDLLEILVPGVSRKP | |
| receptor | SLLIPQGSVVARGGSLTLQCRSDVGYDIFVLYKEGEHDLVQGSGQQPQAGLSQANF | |
| subrfamily B | TLGPVSRSHGGQYRCYGAHNLSPRWSAPSDPLDILIAGLIPDIPALSVQPGPKVAS | |
| member 5 | GENVTLLCQSWHQIDTFFLTKEGAAHPPLCLKSKYQSYRHQAEFSMSPVTSAQGGT | |
| (LILRB5) | YRCYSAIRSYPYLLSSPSYPQELVVSGPSGDPSLSPTGSTPTPGPEDQPLTPTGLD | |
| O75023 | PQSGLGRHLGVVTGVSVAFVLLLFLLLFLLLRHRHQSKHRTSAHFYRPAGAAGPEP | |
| (incl. signal | KDQGLQKRASPVADIQEEILNAAVKDTQPKDGVEMDARAAASEAPQVTYAGLHSL | |
| peptide | TLRREATEPPPSQEREPPAEPSIYAPLAIH | |
| (underlined)) | | |
| Human | MPLKHYLLLLVGCQAWGAGLAYHGCPSECTCSRASQVECTGARIVAVPTPLPWNAM | 16 |
| Leucine-rich | SLQILNTHITELNESPFLNISALIARIEKNELSRITPGAFRNLGSLRYLSLANNK | |
| repeat- | LQVLPIGLFQGLDSLESLLLSSNQLLQIQPAHFSQCSNLKELQLHGNHLEYIPDGA | |
| containing | FDHLVGLTKLNLNGKNSLTHISPRVFQHLGNLQVLRLYENRLTDIPMGTFDGLVNLQ | |
| protein 15 | ELALQQNQIGLLSPGLFHNNHNLQRLYLSNNHISQLPPSVFMQLPQLNRLTLFGNS | |
| (LRRC15) | LKELSPGIFGPMPNLRELWLYDNHISSLPDNVFSNLRQLQVLILSRNQISFISPGA | |
| Q8TF66 | FNGLTELRELSLHTNALQDLDGNVFRMLANLQNISLQNNRLRQLPGNIFANVNGLM | |
| (incl. signal | AIQLQNNQLENLPLGIFDHLGKLCELRLYDNPWRCDSDIILPLRNWLLLNQPRLGTD | |
| peptide | TVPVCFSPANVRGQSLIIINVNVAVPSVHVPEVPSYPETPWYPDTPSYPDTTSVSS | |
| (underlined)) | TTELTSPVEDYTDLTTIQVTDDRSVWGMTQAQSGLAIAAIVIGIVALACSLAACVG | |
| | CCCCKKRSQAVLMQMKAPNEC | |
| Human | MGFHLITQLKGMSVVLVLLPTLLLVMLTGAQRACPKNCRCDGKIVYCESHAFADIP | 17 |
| Leucine-rich | ENISGGSQGLSLRFNSIQKLKSNQFAGLNQLIWLYLDHNYISSVDEDAFQGIRRLK | |
| repeat | ELILSSNKITYLHNKTFHPVPNLRNLDLSYNKLQTLQSEQFKGLRKLIILHLRSNS | |
| transmembrane | LKTVPIRVFQDCRNLDFLDLGYNRLRSLSRNAFAGLLKLKELHLEHNQFSKINFAH | |
| neuronal | FPRLFNLRDIYLQWNRIRSISQGLTWTWSSLHNLDLSGNDIQGIEPGTFKCLPNLQ | |
| protein 4 | KLNLDSNKLTNISQETVNAWISLISTITLSGNWECSRSICPLFYWLKNFKGNKEST | |
| (LRRTM4) | MICAGPKHIQGEKVSDAVETYNICSEVQVVNTERSHLVPQTPQKPLIIPRPTIFKP | |
| Q86VH4 | CFTQSTFETPSPSPGFQIPGAEQEYEHVSFHKIIAGSVALFLSVAMILLVIYVSWK | |
| (incl. signal | RYPASMKQLQQHSLMKRRRKKARESERQMNSPLQEYYVDYKPTNSETMDISVNGSG | |
| peptide | PCTYTISGSRECEMPHHMKPLPYYSYDQPVIGYCQAHQPLHVTKGYETVSPEQDES | |
| (underlined)) | PGLELGRDHSFIATIARSSPAIYLERIAN | |
| Human NPDC1 | MATPLPPPSPRHLRLLRLLLSGLVLGAALRGAAAGHPDVAACPGSLDCALKRRARC | 18 |
| Q9NQX5 | PPGAHACGPCLQPFQEDQQGLCVPRMRRPPGGGRPQPRLEDEIDFLAQELARKESG | |
| (incl. signal | HSTPPLPKDRQRLPEPATLGFSARGQGLELGLPSTPGTPTPTPHTSLGSPVSSIDPV | |
| peptide | HMSPLEPRGGQGDGLALVLILAFCVAGAAALSVASLCWCRLQREIRLTQKADYATA | |
| (underlined)) | KAPGSPAAPRISPGDQRLAQSAEMYHYQHQRQQMLCLERHKEPPKELDTASSDEEN | |
| | EDGDFTVYECPGLAPTGEMEVRNPLFDHAALSAPLPAPSSPPALP | |
| HUMAN PILR | MESRMWPALLLSHLLPLWPLLLLPLPPPAQGSSSSPRTPPAPARPPCARGGPSAPR | 19 |
| alpha- | HVCVWERAPPPSRSPRVPRSRRQVLPGTAPPATPSGFEEGPPSSQYPWAIVWGPTV | |
| associated | SREDGGDPNSANPGFLDYGFAAPHGLATPHPNSDSMRGDGDGLILGEAPATLRPFL | |
| neural | FGGRGEGVDPQLYVTITISIIIVLVATGIIFKFCWDRSQKRRRPSGQQGALRQEES | |
| protein | QQPLTDLSPAVTVLGAFGDSPTPTPDHEEPRGGPRPGMPHPKGAPAFQLNRIPLV | |
| (PIANP) | NL | |
| Q8IYJ0 | | |
| (incl. signal | | |
| peptide | | |
| (underlined)) | | |
| HUMAN Serine | MLLFSVLLLLSLVTGTQLGPRTPLPEAGVAILGRARGAHRPQPPHPPSPVSECGDR | 20 |
| protease 55 | SIFEGRTRYSRITGGMEAEVGEFPWQVSIQARSEPFCGGSILNKWWILTAAHCLYS | |
| (PRSS55) | EELFPEELSVVLGTNDLTSPSMEIKEVASIILHKDFKRANMDNDIALLLLASPIKL | |
| Q6UWB4 | DDLKVPICLPTQPGPATWRECWVAGWGQTNAADKNSVKTDLMKAPMVIMDWEECSK | |
| (incl. signal | MFPKLTKNMLCAGYKNESYDACKGDSGGPLVCTPEPGEKWYQVGIISWGKSCGEKN | |
| peptide | TPGIYTSLVNYNLWIEKVTQLEGRPFNAEKRRTSVKQKPMGSPVSGVPEPGSPRSW | |
| (underlined)) | LLLCPLSHVLFRAILY | |
| Forward | GCGGCCTTGTGCTGTAGAA | 21 |
| Primer Seq | | |
| Reverse | GCTCCCGACGTGAGAATATCC | 22 |
| Primer Seq | | |
| Reporter 1 | VIC-ACTTCCACGGGCAGTC-NFQ | 23 |
| Seq | | |
| Reporter 2 | FAM-ACTTCCACAGGCAGTC-NFQ | 24 |
| Seq | | |
| Forward | GGCCTGGCTATCCGGAGA | 25 |
| Primer Seq | | |
| Reverse | GCGCAGAGACATCGCGA | 26 |
| Primer Seq | | |
| HSV-1 Probe | FAM-CAGCACACGACTTGGCGTTCTGTGT-MGB | 27 |
| 12C6.9 | KASQNVGTKVA | 28 |
| HVR-L1 | | |
| 12C6.9 | SASYRFS | 29 |
| HVR-L2 | | |
| 12C6.9 | QQYNTYPLT | 30 |
| HVR-L3 | | |
| 12C6.9 | TYGMS | 31 |
| HVR-H1 | | |
| 12C6.9 | WINTYSGVPTYADDFKG | 32 |
| HVR-H2 | | |
| 12C6.9 | RDYGSSQWYFDV | 33 |
| HVR-H3 | | |
| 12H1.8 | RASQDVNTAVA | 34 |
| HVR-L1 | | |
| 12H1.8 | SASYRY | 35 |
| HVR-L2 | | |
| 12H1.8 | QQHYTTPLT | 36 |
| HVR-L3 | | |
| 12H1.8 | NYWIG | 37 |
| HVR-H1 | | |
| 12H1.8 | DIYPGGGYTNYNKKFKG | 38 |
| HVR-H2 | | |
| 12H1.8 | SRGHGSNFYWYFDV | 39 |
| HVR-H3 | | |
| 12D4 | KASQDVSTAVA | 40 |
| HVR-L1 | | |
| 12D4 | SASYRYT | 41 |
| HVR-L2 | | |
| 12D4 | QQHYSTPLT | 42 |
| HVR-L3 | | |
| 12D4 | SYWMN | 43 |
| HVR-H1 | | |
| 12D4 | WIYGGSGNTKYNQKFQG | 44 |
| HVR-H2 | | |
| 12D4 | GTNFFDY | 45 |
| HVR-H3 | | |
| 12C6.9 | DIVMTQSPKFMSISVGDRVSVTCKASQNVGTKVAWYQQKPGQSPKEILIYSASYRFS | 46 |
| Light Chain | GVPDRFTGSGSGTDFTLTISSVQSEDLAEYFCQQYNTYPLTFGAGTKLEIK | |
| Variablle | | |
| Region (VL) | | |
| 12C6.9 | QIQLVQSGPELKKPGETVKISCKASGYTFTTYGMSWMKQAPGKGLKWMGWINTYSG | 47 |
| Heavy Chain | VPTYADDFKGRFAFSLETSASTAYLQISNLKDEDTARYFCARRDYGSSQWYFDVWS | |
| Variable | TGTTVTVSS | |
| Region (VH) | | |
| 12H1.8 | DIVMTQSPKFMSTSVGDRVNITCRASQDVNTAVAWFQQKPGRSPKLLIYSASYRYT | 48 |
| Light Chain | GPDHFTGSGSGTDFTFTISSVQAEDLAVYYCQQHYTTPLTFGAGTKLEIK | |
| Variable | | |
| Region (VL) | | |
| 12H1.8 | QVQLQQSGAELVGPGPTSVKISCQASGYTFTNYWIEWAKQRPGHGLEWIGDIYPGGG | 49 |
| Heavy Chain | YTNYNKKFKGKATLTADKSSSTAYMQFSSLTSEDSAIYYCSRSRGHGSNFYWYFDV | 49 |
| Variable | WGTGTTVTVSS | |
| Region (VH) | | |
| 12D4 | DIVMTQSHKFMSTSVGDRVSITCKASQDVSTAVAWYQQKPGQSPKLLIYSASYRYT | 50 |
| Light Chain | GVPDRFTGSGSGTDFTFTISSVQAEDLAVYYCQQHYSTPLTFGAGTKLELK | |
| Variable | | |
| Region (VL) | | |
| 12D4 | QVQLQQSGAELVTPGASVKLSCKTSGHTFTSYWMNWVNQKPGQGLEWIGWIYGGSG | 51 |
| Heavy Chain | NTKYNQKFQGKATLTVDTSSSTAYMELRSLTSDDSAVYFCASGTNFFDYWGQGTMV | |
| Variable | TVSS | |
| Region (VH) | | |
| 12C6.9 | DIVMTQSPKFMSISVGDRVSVTCKASQNVGTKVAWYQQKPGQSPKELIYSASYRFS | 52 |
| Light Chain | GVPDRFTGSGSGTDFTLTISSVQSEDLAEYFCQQYNTYPLTFGAGTKLEIKRADAA | |
| (LC) | PTVSIFPPSSEQLTSGGASVVCFLNNFYPKDINVKWKDGSERQNGVLNSWTDQDS | |
| | KDSTYSMSSTLT LTKDEYERHNSYTCEATHKTSTSPIVKSFNRNEC | |
| 12C6.9 | QIQLVQSGPELKKPGETVKISCKASGYTFTTYGMSWMKQAPGKGLKWMGWINTYSG | 53 |
| Heavy Chain | VPTYADDFKGRFAFSLETSASTAYLQISNLKDEDTARYFCARRDYGSSQWYFDVWS | |
| (HC) | TGTTVTVSSAKTTAPSVYPLAPVCGDTTGSSVTLGCLVKGYFPEPVTLTWNSGSLS | |
| | SGVHTFPAVLQSDLYTLSSSVTVTSSTWPSQSITCNVAHPASSTKVDKKIEPRGPT | |
| | IKPCPPCKCPAPNLLGGPSVFIFPPKIKDVLMISLSPIVTCVVVDVSEDDPDVQIS | |
| | WFVNNVEVHTAQTQTHREDYNSTLRVVSALTIQHQDWMSGKEFKCKVNNKDLPAPI | |
| | ERTISKPKGSVRAPQVYVLPPPEEEMTKKQVTLTCMVTDFMPEDIYVEWTNNGKTE | |
| | LNYKNTEPVLDSDGSYFMYSKLRVEKKNWVERNSYSCSVVHEGLHNHHTTKSFSRT | |
| | PGK | |
| 12H1.8 | DIVMTQSPKFMSTSVGDRVNITCRASQDNTAVAWFQQKPGRSPKLLIYSASYRYT | 54 |
| Light Chain | GPDHFTGSGSGTDFTFTISSVQAEDLAVYYCQQHYTTPLTFGAGTKLEIKRADAAP | |
| (LC) | TVSIFPPSSEQLTSGGASVVCFLNNFYPKDINVKWKIDGSERQNGVLNSWTDQDSK | |
| | DSTYSMSSTLT LTKDEYERHNSYTCEATHKTSTSPIVKSFNRNEC | |
| 12H1.8 | QVQLQQSGAELVGPGTSVKISCQASGYTFTNYWIGWAKQRPGHGLEWIGDIYPGGG | 55 |
| Heavy Chain | YTNYNKKFKGKATLTADKSSSTAYMQFSSLTSEDSAIYYCSRSRGHGSNFYWYFDV | |
| (VH) | WGTGTTVTVSSAKTTAPSVYPLAPVCGDTTGSSVTLGCLVKGYFPEPVTLTWNSGS | |
| | LSSGVHTFPAVLQSDLYTLSSSVTVTSSTWPSQSITCNVAHPASSTKVDKKIEPRG | |
| | PTIKPCPPCKCPAPNLIGGPSVFIFPPKIKDVLMISLSPIVTCVVVDVSEDDPDVQ | |
| | ISWFVNNVEVHTAQTQTHREDYNSTLRVVSALPIQHQDWMSGKEKFKCKVNNKDLFA | |
| | PIERTISKPKGSVRAPQVYVLPPPEEEMTKKQVTLTCMVTDFMPEDIYVEWTNNGK | |
| | TELNYKNTEPVLDSDGSYFMYSKLRVEKKNWVERNSYSCSVVHEGLHNHHTTKSFS | |
| | RTPGK | |
| 12D4 | DIVMTQSHKFMSTSVGDRVSITCKASQDVSTAVAWYQQKPGQSPKLLIYSASYRYT | 56 |
| Light Chain | GVPDRFTFSFSFTDFTFTISSVQAEDLAVYYCQQHYSTPLTFGAGTKLELKRADAA | |
| (LC) | PTVSIFPPSSEQLTSGGASVVCFLNNFYPKDINVKWKIDGSERQNGVLNSWTDQDS | |
| | KDSTYSMSSTLT LTKDEYERHNSYTCEATHKTSTSPIVKSFNRNEC | |
| 12E4 | QVQLQQSGAELVTPGASVKLSCKTSGHTFTSYWMNWVNQKPGQGLEWIGWIYGGSG | 57 |
| Heavy Chain | NTKYNQKFQGKATLTVDTSSSTAYMELRSLTSDDSAVYFCASGTNFFDYWGQGTMV | |
| (HC) | TVSSAKTTAPSVYPLAPVCGDTTGSSVTLGCLVKGYFPEPVTLTWNSGSLSSGVHT | |
| | FPAVLQSDLYTLSSSVTVTSSTWPSQSITCNVAHPASSTKVDKKIEPRGPTIKPCP | |
| | PCKCPAPNLLGGPSVFIFPPKIKDVLMISLSPIVTCVVVDVSEDDPDVQISWFVNN | |
| | VEVHTAQTQTHREDYNSTLRVVSALPIQHQDWMSGKEFKCKVNNKDLPAPIERTIS | |
| | KPKGSVRAPQVYVLPPPEEEMTKKQVTLTCMVTDFMPEDIYVEWTNNGKTELNYKN | |
| | TEPVLDSDGSYFMYSKLRVEKKNWVERNSYSCSVVHEGLHNHHTTKSFSRTPGK | |
| C99L2_MOUSE | MVARLTAFLVCLVFSLATLVQRGYGDTDGFNLEDALKETSSVKQRWDHFSTTTRRP | 58 |
| CD99 antigen- | VTTRAPANPAERWDHVATTTTRRPGTTRAPSNPMELDGFDLEDALDDRNDLDGPKK | |
| like protein | PSAGEAGGWSDKDLEDIVEGGGYKPDKNKGGGGYGSNDDPGSGISTETGTIAGVAS | |
| 2 | ALAMALIGAVSSYISYQQKKFCFSIQQGLNADYVKGENLEAVVCEEPQVTYSKQET | |
| Q8BIF0 | QSAEPPPPEPPRI | |
| (incl. signal | | |
| peptide | | |
| (underlined)) | | |
| C99L2_HUMAN | MVAWRSAFLVCLAFSLATLVQRGSGDFDDFNLEDAVKETSSVKQPWDHTTTTTNR | 59 |
| CD99 antien- | PGTTRAPAKPPGSGLDLADALDDQDDGRRKPGIGGRERWNHVTTTTKRPVTTRAPA | |
| like protein | NTLGNDFDLADALDDRNDRDDGRRKPIAGGGGFSDKDLEDIVGGGEYKPDKGKGDG | |
| 2 | RYGSNDDPGSGMVAEPGTIAGVASALAMALIGAVSSYISYQQKKFCFSIQQGLNAD | |
| Q8TCZ2 | YVKGENLEAVVCEEPQVKYSTLHTQSAEPPPPPEPARI | |
| (incl. signal | | |
| peptide | | |
| (underlined)) | | |
| PIANP_MOUSE | MWSAQLLSQLLPLWPLLLLSVLPPAQGSSHRSPPAPARPPCVRGGPSAPRHVCVWE | 60 |
| PILR alphs- | RAPPPSRSPRVPRSRRQVVPGTAPPATPSGFEEGPPSSQYPWAIVWGPTVSREDGG | |
| associated | DPNSVNPGFLPLDYGFAAPHGLATPHPNSDSMRDDGDGLILGETPATLRPFLFGGR | |
| neural | GEGVDPQLYVTITISIIIVLVATGIIFKFCWDRSQKRRRPSGQQGALRQEESGGPL | |
| protein | TDLSPAGVTVLGAFGDSPTPTPDHEEPRGGPRPGMPQPKGAPAFQLNRIPLVNL | |
| Q6P1B3 | | |
| (incl signal | | |
| peptide | | |
| (underlined)) | | |
| PIRA_MOUSE | MALLISLPGGTPAMAQILLLLSSACLHAGNSERSNRKNGFGVNQPESCSGVQGGSI | 61 |
| Paired | DIPFSFYFPWKLAKDPQMSIAWRWKDFHGEFIYNSSLPFIHEHFKGRLILNWTQGQ | |
| immunoglobuline- | TSGVLRILNLKESDQTRYFGRVFLQTTEGIQFWQSIPGTQLNVTNATCTPTTLPST | |
| like type 2 | TAATSAHTQNDITEVKSANIGGLDLQTTVGLATAAVFLVGVLGLIVFLWWKRRRQ | |
| receptor | GQKTKAEIPAREPLETSEKHESVGHEGQCMDPKENPKDNNIVYASISLSSPTSPGT | |
| alphs | APNLPVHGNPQEETVYSIVKAK | |
| Q2YFS3 | | |
| (incl. signal | | |
| peptide | | |
| (underlined)) | | |

1. A method for treating a disease associated with myeloid cell dysfunction in a subject comprising administering an effective amount of an agent to the subject, wherein the agent specifically binds to one or more variants of Paired Immunoglobulin-like Type 2 Receptor Alpha (PILRA) thereby inhibiting the interaction between PILRA and any one of its ligands.

2. A method of selecting a subject having a disease associated with myeloid cell dysfunction for a treatment with an agent inhibiting the interaction between one or more variants of PILRA and any one of its ligands, comprising determining the presence or absence of the one or more variants of PILRA in a biological sample from the subject, wherein the presence of the one or more variants of PILRA indicates that the subject is suitable for treatment with the agent.

3. A method of predicting the response of a subject having a disease associated with myeloid cell dysfunction to a treatment with an agent specifically binding to one or more variants of PILRA, the method comprising:
(a) measuring whether the agent specifically binding to the one or more variants of PILRA inhibits the interaction between PILRA and any one of its ligands as compared to a reference level, and
(b) predicting that the subject will respond to the treatment when the interaction between PILRA and any one of its ligands is inhibited as compared to the reference level and predicting that the subject will not respond to the treatment when the interaction between PILRA and any one of its ligands is not inhibited as compared to the reference level.

4. A method for detecting the presence or absence of one or more variants of PILRA indicating that a subject having a disease associated with myeloid cell dysfunction is suitable for treatment with an agent inhibiting the interaction between PILRA and any one of its ligands, comprising:
(a) contacting a sample from the subject with a reagent capable of detecting the presence or absence of the one more variants of PILRA; and
(b) determining the presence or absence of the one or more variants of PILRA, wherein the presence of the one or more variants of PILRA indicates that the subject is suitable for treatment with an agent inhibiting the interaction between PILRA and any one of its ligands.

5. A method for selecting an agent for treating a disease associated with myeloid cell dysfunction, comprising determining whether the agent inhibits the interaction between PILRA and any one of its ligands, wherein the agent that inhibits the interaction between PILRA and any one of its ligands is suitable for treating the disease associated with myeloid cell dysfunction.

6. The method of claim 1, wherein the disease associated with myeloid cell dysfunction is selected from the group consisting of Alzheimer's Disease (AD) and Herpes Simplex Virus-1 (HSV-1) infection.

7. The method of claim 1, wherein the myeloid cell dysfunction is associated with decreased myeloid cell activity.

8. The method of claim 1, wherein the one or more variants of PILRA are encoded by a polynucleotide sequence comprising one or more SNPs.

9. The method of claim 8, wherein the one or more SNPs result in one or a combination of the following amino acids at the given positions:
i) the amino acid glycine or arginine at position 78;
ii) the amino acid serine or leucine at position 279; of the full-length unprocessed PILRA.

10. The method of claim 9, wherein the SNP results in the amino acid arginine at position 78 of the full-length unprocessed PILRA.

11. The method of claim 10, wherein the SNP is rs1859788.

12. The method of claim 1, wherein the agent stabilizes the non-ligand bound form of the PILRA receptor.

13. The method of claim 1, wherein the agent reduces the inhibitory signaling in myeloid cells.

14. The method of claim 1, wherein the agent inhibits the interaction between PILRA and any one of its ligands by binding to one or more amino acids on PILRA.

15. The method of claim 14, wherein the one or more amino acids are located within the sialic acid (SA) binding region of PILRA.

16. The method of claim 15, wherein the one or more amino acids are selected from the group consisting of Y33, R126, T131, R132, Q138, W139 and Q140 of the full-length unprocessed PILRA.

17. The method of claim 16, wherein the one or more amino acids are R126 and/or Q140 of the full-length unprocessed PILRA.

18. The method of claim 1, wherein the agent inhibits the interaction between PILRA and any one of its ligands by at least 50% as compared to a reference level.

19. The method of claim 1, wherein the reference level is based on the interaction between the G78 variant of PILRA and any one of its ligands.

20. The method of claim 1, wherein the agent decreases infection of a myeloid cell during HSV-1 recurrence.

21. The method of claim 1, wherein the myeloid cell is a CNS resident myeloid cell.

22. The method of claim 21, wherein the CNS resident myeloid cell is selected from the group consisting of microglia, perivascular macrophages, meningeal macrophages, and choroid plexus macrophages.

23. The method of claim 22, wherein the CNS resident myeloid cell is a microglia.

24. The method of claim 1, wherein the agent is selected from the group consisting of an antibody, a polypeptide, a polynucleotide, and a small molecule.

25. The method of claim 1, wherein the agent is an antibody.

26. The method of claim 25, wherein the antibody is a monoclonal antibody.

27. The method of claim 26, wherein the monoclonal antibody is a human, humanized, or chimeric antibody.

28. The method of claim 24, wherein the antibody is a full length IgG1 antibody.

29. The method of claim 1, wherein the ligand is an endogenous ligand.

30. The method of claim 29, wherein the endogenous ligand is selected from the group consisting of APLP1, C16orf54, C4A, C4B, CD99, CLEC4G, COLEC12, DAG1, EVA1C, FceRII, IL17RA, LILRB5, LRRC15, LRRTM4, NPDC1, PIANP, and PRSS55.

31. The method of claim 1, wherein the ligand is an exogenous ligand.

32. The method of claim 31, wherein the exogenous ligand is HSV-1 glycoprotein B.

33. The method of claim 1, wherein the sample is selected from the group consisting of cerebrospinal fluid, blood, serum, sputum, saliva, mucosal scraping, tissue biopsy, lacrimal secretion, semen, and sweat.

34. The method of claim 1, wherein the subject is a human.

35. An agent specifically binding to one or more variants of PILRA for use in medical treatment or diagnosis including therapy and/or treating of a disease associated with myeloid cell dysfunction.

36-53. (canceled)

54. A pharmaceutical formulation comprising a pharmaceutically active amount of an agent specifically binding to one or more variants of PILRA according to claim 35 and a pharmaceutically acceptable carrier.


SEQUENCE LISTING (71 sequences)

SEQ ID NO: 1 - protein, 303 aa, Homo sapiens
MGRPLLLPLLPLLLPPAFLQPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 2 - protein, 303 aa, Homo sapiens
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 3 - protein, 303 aa, Homo sapiens
MGRPLLLPLLPLLLPPAFLQPSGSTGSGPSYLYGVTQPKHLSASMGGSVEIPFSFYYPWELATAPDVRISWRRGHFHGQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTLPRAPPSHRPLKSPQNETLYSVLKA

SEQ ID NO: 4 - protein, 650 aa, Homo sapiens
MGPASPAARGLSRRPGQPPLPLLLPLLLLLLRAQPAIGSLAGGSPGAAEAPGSAQVAGLCGRLTLHRDLRTGRWEPDPQRSRRCLRDPQRVLEYCRQMYPELQIARVEQATQAIPMERWCGGSRSGSCAHPHHQVVPFRCLPGEFVSEALLVPEGCRFLHQERMDQCESSTRRHQEAQEACSSQGLILHGSGMLLPCGSDRFRGVEYVCCPPPGTPDPSGTAVGDPSTRSWPPGSRVEGAEDEEEEESFPQPVDDYFVEPPQAEEEEETVPPPSSHTLAVVGKVTPTPRPTDGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSEHLGPSELEAPAPGGSSEDKGGLQPPDSKDDTPMTLPKGSTEQDAASPEKEKMNPLEQYERKVNASVPRGFPFHSSEIQRDELAPAGTGVSREAVSGLLIMGAGGGSLIVLSMLLLRRKKPYGAISHGVVEVDPMLTLEEQQLRELQRHGYENPTYRFLEERP

SEQ ID NO: 5 - protein, 224 aa, Homo sapiens
MPLTPEPPSGRVEGPPAWEAAPWPSLPCGPCIPIMLVLATLAALFILTTAVLAERLFRRALRPDPSHRAPTLVWRPGGELWIEPMGTARERSEDWYGSAVPLLTDRAPEPPTQVGTLEARATAPPAPSAPNSAPSNLGPQTVLEVPARSTFWGPQPWEGRPPATGLVSWAEPEQRPEASVQFGSPQARRQRPGSPDPEWGLQPRVTLEQISAFWKREGRTSVGF

SEQ ID NO: 6 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 7 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 8 - protein, 293 aa, Homo sapiens
MDTTRYSKWGGSSEEVPGGPWGRWVHWSRRPLFLALAVLVTTVLWAVILSILLSKASTERAALLDGHDLLRTNASKQTAALGALKEEVGDCHSCCSGTQAQLQTTRAELGEAQAKLMEQESALRELRERVTQGLAEAGRGREDVRTELFRALEAVRLQNNSCEPCPTSWLSFEGSCYFFSVPKTTWAAAQDHCADASAHLVIVGGLDEQGFLTRNTRGRGYWLGLRAVRHLGKVQGYQWVDGVSLSFSHWNQGEPNDAWGRENCVMMLHTGLWNDAPCDSEKDGWICEKRHNC

SEQ ID NO: 9 - protein, 742 aa, Homo sapiens
MKDDFAEEEEVQSFGYKRFGIQEGTQCTKCKNNWALKFSIILLYILCALLTITVAILGYKVVEKMDNVTGGMETSRQTYDDKLTAVESDLKKLGDQTGKKAISTNSELSTFRSDILDLRQQLREITEKTSKNKDTLEKLQASGDALVDRQSQLKETLENNSFLITTVNKTLQAYNGYVTNLQQDTSVLQGNLQNQMYSHNVVIMNLNNLNLTQVQQRNLITNLQRSVDDTSQAIQRIKNDFQNLQQVFLQAKKDTDWLKEKVQSLQTLAANNSALAKANNDTLEDMNSQLNSFTGQMENITTISQANEQNLKDLQDLHKDAENRTAIKFNQLEERFQLFETDIVNIISNISYTAHHLRTLTSNLNEVRTTCTDTLTKHTDDLTSLNNTLANIRLDSVSLRMQQDLMRSRLDTEVANLSVIMEEMKLVDSKHGQLIKNFTILQGPPGPRGPRGDRGSQGPPGPTGNKGQKGEKGEPGPPGPAGERGPIGPAGPPGERGGKGSKGSQGPKGSRGSPGKPGPQGSSGDPGPPGPPGKEGLPGPQGPPGFQGLQGTVGEPGVPGPRGLPGLPGVPGMPGPKGPPGPPGPSGAVVPLALQNEPTPAPEDNGCPPHWKNFTDKCYYFSVEKEIFEDAKLFCEDKSSHLVFINTREEQQWIKKQMVGRESHWIGLTDSERENEWKWLDGTSPDYKNWKAGQPDNWGHGHGPGEDCAGLIYAGQWNDFQCEDVNNFICEKDRETVLSSAL

SEQ ID NO: 10 - protein, 895 aa, Homo sapiens
MRMSVGLSLLLPLSGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSELQSVRTASPDPGEVVSSACAADEPVTVLTVILDADLTKMTPKQRIDLLHRMRSFSEVELHNMKLVPVVNNRLFDMSAFMAGPGNAKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTAPMEGKGSRPKNMTPYRSPPPYVPP

SEQ ID NO: 11 - protein, 441 aa, Homo sapiens
MLLPGRARQPPTPQPVQHPGLRRQVEPPGQLLRLFYCTVLVCSKEISALTDFSGYLTKLLQNHTTYACDGDYLNLQCPRHSTISVQSAFYGQDYQMCSSQKPASQREDSLTCVAATTFQKVLDECQNQRACHLLVNSRVFGPDLCPGSSKYLLVSFKCQPNELKNKTVCEDQELKLHCHESKFLNIYSATYGRRTQERDICSSKAERLPPFDCLSYSALQVLSRRCYGKQRCKIIVNNHHFGSPCLPGVKKYLTVTYACVPKNILTAIDPAIANLKPSLKQKDGEYGINFDPSGSKVLRKDGILVSNSLAAFAYIRAHPERAALLFVSSVCIGLALTLCALVIRESCAKDFRDLQLGREQLVPGSDKVEEDSEDEEEEEDPSESDFPGELSGFCRTSYPIYSSIEAAELAERIERREQIIQEIWMNSGLDTSLPRNMGQFY

SEQ ID NO: 12 - protein, 321 aa, Homo sapiens
MEEGQYSEIEELPRRRCCRRGTQIVLLGLVTAALWAGLLTLLLLWHWDTTQSLKQLEERAARNVSQVSKNLESHHGDQMAQKSQSTQISQELEELRAEQQRLKSQDLELSWNLNGLQADLSSFKSQELNERNEASDLLERLREEVTKLRMELQVSSGFVCNTCPEKWINFQRKCYYFGKGTKQWVHARYACDDMEGQLVSIHSPEEQDFLTKHASHTGSWIGLRNLDLKGEFIWVDGSHVDYSNWAPGEPTSRSQGEDCVMMRGSGRWNDAFCDRKLGAWVCDRLATCTPPASEGSAESMGPDSRPDPDGRLPTPSAPLHS

SEQ ID NO: 13 - protein, 904 aa, Herpes simplex virus 1
MHQGAPSWGRRWFVVWALLGLTLGVLVASAAPTSPGTPGVAAATQAANGGPATPAPPPLGAAPTGDPKPKKNKKPKNPTPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTTYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYQANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASVTVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 14 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 15 - protein, 590 aa, Homo sapiens
MTLTLSVLICLGLSVGPRTCVQAGTLPKPTLWAEPASVIARGKPVTLWCQGPLETEEYRLDKEGLPWARKRQNPLEPGAKAKFHIPSTVYDSAGRYRCYYETPAGWSEPSDPLELVATGFYAEPTLLALPSPVVASGGNVTLQCDTLDGLLTFVLVEEEQKLPRTLYSQKLPKGPSQALFPVGPVTPSCRWRFRCYYYYRKNPQVWSNPSDLLEILVPGVSRKPSLLIPQGSVVARGGSLTLQCRSDVGYDIFVLYKEGEHDLVQGSGQQPQAGLSQANFTLGPVSRSHGGQYRCYGAHNLSPRWSAPSDPLDILIAGLIPDIPALSVQPGPKVASGENVTLLCQSWHQIDTFFLTKEGAAHPPLCLKSKYQSYRHQAEFSMSPVTSAQGGTYRCYSAIRSYPYLLSSPSYPQELVVSGPSGDPSLSPTGSTPTPGPEDQPLTPTGLDPQSGLGRHLGVVTGVSVAFVLLLFLLLFLLLRHRHQSKHRTSAHFYRPAGAAGPEPKDQGLQKRASPVADIQEEILNAAVKDTQPKDGVEMDARAAASEAPQDVTYAQLHSLTLRREATEPPPSQEREPPAEPSIYAPLAIH

SEQ ID NO: 16 - protein, 581 aa, Homo sapiens
MPLKHYLLLLVGCQAWGAGLAYHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSGLAIAAIVIGIVALACSLAACVGCCCCKKRSQAVLMQMKAPNEC

SEQ ID NO: 17 - protein, 590 aa, Homo sapiens
MGFHLITQLKGMSVVLVLLPTLLLVMLTGAQRACPKNCRCDGKIVYCESHAFADIPENISGGSQGLSLRFNSIQKLKSNQFAGLNQLIWLYLDHNYISSVDEDAFQGIRRLKELILSSNKITYLHNKTFHPVPNLRNLDLSYNKLQTLQSEQFKGLRKLIILHLRSNSLKTVPIRVFQDCRNLDFLDLGYNRLRSLSRNAFAGLLKLKELHLEHNQFSKINFAHFPRLFNLRSIYLQWNRIRSISQGLTWTWSSLHNLDLSGNDIQGIEPGTFKCLPNLQKLNLDSNKLTNISQETVNAWISLISITLSGNMWECSRSICPLFYWLKNFKGNKESTMICAGPKHIQGEKVSDAVETYNICSEVQVVNTERSHLVPQTPQKPLIIPRPTIFKPDVTQSTFETPSPSPGFQIPGAEQEYEHVSFHKIIAGSVALFLSVAMILLVIYVSWKRYPASMKQLQQHSLMKRRRKKARESERQMNSPLQEYYVDYKPTNSETMDISVNGSGPCTYTISGSRECEMPHHMKPLPYYSYDQPVIGYCQAHQPLHVTKGYETVSPEQDESPGLELGRDHSFIATIARSAAPAIYLERIAN

SEQ ID NO: 18 - protein, 325 aa, Homo sapiens
MATPLPPPSPRHLRLLRLLLSGLVLGAALRGAAAGHPDVAACPGSLDCALKRRARCPPGAHACGPCLQPFQEDQQGLCVPRMRRPPGGGRPQPRLEDEIDFLAQELARKESGHSTPPLPKDRQRLPEPATLGFSARGQGLELGLPSTPGTPTPTPHTSLGSPVSSDPVHMSPLEPRGGQGDGLALVLILAFCVAGAAALSVASLCWCRLQREIRLTQKADYATAKAPGSPAAPRISPGDQRLAQSAEMYHYQHQRQQMLCLERHKEPPKELDTASSDEENEDGDFTVYECPGLAPTGEMEVRNPLFDHAALSAPLPAPSSPPALP

SEQ ID NO: 19 - protein, 282 aa, Homo sapiens
MESRMWPALLLSHLLPLWPLLLLPLPPPAQGSSSSPRTPPAPARPPCARGGPSAPRHVCVWERAPPPSRSPRVPRSRRQVLPGTAPPATPSGFEEGPPSSQYPWAIVWGPTVSREDGGDPNSANPGFLDYGFAAPHGLATPHPNSDSMRGDGDGLILGEAPATLRPFLFGGRGEGVDPQLYVTITISIIIVLVATGIIFKFCWDRSQKRRRPSGQQGALRQEESQQPLTDLSPAGVTVLGAFGDSPTPTPDHEEPRGGPRPGMPHPKGAPAFQLNRIPLVNL

SEQ ID NO: 20 - protein, 352 aa, Homo sapiens
MLLFSVLLLLSLVTGTQLGPRTPLPEAGVAILGRARGAHRPQPPHPPSPVSECGDRSIFEGRTRYSRITGGMEAEVGEFPWQVSIQARSEPFCGGSILNKWWILTAAHCLYSEELFPEELSVVLGTNDLTSPSMEIKEVASIILHKDFKRANMDNDIALLLLASPIKLDDLKVPICLPTQPGPATWRECWVAGWGQTNAADKNSVKTDLMKAPMVIMDWEECSKMFPKLTKNMLCAGYKNESYDACKGDSGGPLVCTPEPGEKWYQVGIISWGKSCGEKNTPGIYTSLVNYNLWIEKVTQLEGRPFNAEKRRTSVKQKPMGSPVSGVPEPGSPRSWLLLCPLSHVLFRAILY

SEQ ID NO: 21 - DNA, 19 bp, Artificial Sequence
  Description of Artificial Sequence Synthetic primer
gcggccttgtgctgtagaa

SEQ ID NO: 22 - DNA, 21 bp, Artificial Sequence
  Description of Artificial Sequence Synthetic primer
gctcccgacgtgagaatatcc

SEQ ID NO: 23 - DNA, 16 bp, Artificial Sequence
  Description of Artificial Sequence Synthetic oligonucleotide
acttccacgggcagtc

SEQ ID NO: 24 - DNA, 16 bp, Artificial Sequence
  Description of Artificial Sequence Synthetic oligonucleotide
acttccacaggcagtc

SEQ ID NO: 25 - DNA, 18 bp, Artificial Sequence
  Description of Artificial Sequence Synthetic primer
ggcctggctatccggaga

SEQ ID NO: 26 - DNA, 17 bp, Artificial Sequence
  Description of Artificial Sequence Synthetic primer
gcgcagagacatcgcga

SEQ ID NO: 27 - DNA, 25 bp, Artificial Sequence
  Description of Artificial Sequence Synthetic probe
cagcacacgacttggcgttctgtgt

SEQ ID NO: 28 - protein, 11 aa, Artificial Sequence
  Description of Artificial Sequence Synthetic peptide
KASQNVGTKVA

SEQ ID NO: 29 - protein, 7 aa, Artificial Sequence
  Description of Artificial Sequence Synthetic peptide
SASYRFS

SEQ ID NO: 30 - protein, 9 aa, Artificial Sequence
  Description of Artificial Sequence Synthetic peptide
QQYNTYPLT

SEQ ID NO: 31 - protein, 5 aa, Artificial Sequence
  Description of Artificial Sequence Synthetic peptide
TYGMS

SEQ ID NO: 32 - protein, 17 aa, Artificial Sequence
  Description of Artificial Sequence Synthetic peptide
WINTYSGVPTYADDFKG

SEQ ID NO: 33 - protein, 12 aa, Artificial Sequence
  Description of Artificial Sequence Synthetic peptide
RDYGSSQWYFDV

SEQ ID NO: 34 - protein, 11 aa, Artificial Sequence
  Description of Artificial Sequence Synthetic peptide
RASQDVNTAVA

SEQ ID NO: 35 - protein, 6 aa, Artificial Sequence
  Description of Artificial Sequence Synthetic peptide
SASYRY

SEQ ID NO: 36 - protein, 9 aa, Artificial Sequence
  Description of Artificial Sequence Synthetic peptide
QQHYTTPLT

SEQ ID NO: 37 - protein, 5 aa, Artificial Sequence
  Description of Artificial Sequence Synthetic peptide
NYWIG

SEQ ID NO: 38 - protein, 17 aa, Artificial Sequence
  Description of Artificial Sequence Synthetic peptide
DIYPGGGYTNYNKKFKG

SEQ ID NO: 39 - protein, 14 aa, Artificial Sequence
  Description of Artificial Sequence Synthetic peptide
SRGHGSNFYWYFDV

SEQ ID NO: 40 - protein, 11 aa, Artificial Sequence
  Description of Artificial Sequence Synthetic peptide
KASQDVSTAVA

SEQ ID NO: 41 - protein, 7 aa, Artificial Sequence
  Description of Artificial Sequence Synthetic peptide
SASYRYT

SEQ ID NO: 42 - protein, 9 aa, Artificial Sequence
  Description of Artificial Sequence Synthetic peptide
QQHYSTPLT

SEQ ID NO: 43 - protein, 5 aa, Artificial Sequence
  Description of Artificial Sequence Synthetic peptide
SYWMN

SEQ ID NO: 44 - protein, 17 aa, Artificial Sequence
  Description of Artificial Sequence Synthetic peptide
WIYGGSGNTKYNQKFQG

SEQ ID NO: 45 - protein, 7 aa, Artificial Sequence
  Description of Artificial Sequence Synthetic peptide
GTNFFDY

SEQ ID NO: 46 - protein, 107 aa, Artificial Sequence
  Description of Artificial Sequence Synthetic polypeptide
DIVMTQSPKFMSISVGDRVSVTCKASQNVGTKVAWYQQKPGQSPKELIYSASYRFSGVPDRFTGSGSGTDFTLTISSVQSEDLAEYFCQQYNTYPLTFGAGTKLEIK

SEQ ID NO: 47 - protein, 121 aa, Artificial Sequence
  Description of Artificial Sequence Synthetic polypeptide
QIQLVQSGPELKKPGETVKISCKASGYTFTTYGMSWMKQAPGKGLKWMGWINTYSGVPTYADDFKGRFAFSLETSASTAYLQISNLKDEDTARYFCARRDYGSSQWYFDVWSTGTTVTVSS

SEQ ID NO: 48 - protein, 106 aa, Artificial Sequence
  Description of Artificial Sequence Synthetic polypeptide
DIVMTQSPKFMSTSVGDRVNITCRASQDVNTAVAWFQQKPGRSPKLLIYSASYRYTGPDHFTGSGSGTDFTFTISSVQAEDLAVYYCQQHYTTPLTFGAGTKLEIK

SEQ ID NO: 49 - protein, 123 aa, Artificial Sequence
  Description of Artificial Sequence Synthetic polypeptide
QVQLQQSGAELVGPGTSVKISCQASGYTFTNYWIGWAKQRPGHGLEWIGDIYPGGGYTNYNKKFKGKATLTADKSSSTAYMQFSSLTSEDSAIYYCSRSRGHGSNFYWYFDVWGTGTTVTVSS

SEQ ID NO: 50 - protein, 107 aa, Artificial Sequence
  Description of Artificial Sequence Synthetic polypeptide
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPLTFGAGTKLELK

SEQ ID NO: 51 - protein, 116 aa, Artificial Sequence
  Description of Artificial Sequence Synthetic polypeptide
QVQLQQSGAELVTPGASVKLSCKTSGHTFTSYWMNWVNQKPGQGLEWIGWIYGGSGNTKYNQKFQGKATLTVDTSSSTAYMELRSLTSDDSAVYFCASGTNFFDYWGQGTMVTVSS

SEQ ID NO: 52 - protein, 214 aa, Artificial Sequence
  Description of Artificial Sequence Synthetic polypeptide
DIVMTQSPKFMSISVGDRVSVTCKASQNVGTKVAWYQQKPGQSPKELIYSASYRFSGVPDRFTGSGSGTDFTLTISSVQSEDLAEYFCQQYNTYPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 53 - protein, 451 aa, Artificial Sequence
  Description of Artificial Sequence Synthetic polypeptide
QIQLVQSGPELKKPGETVKISCKASGYTFTTYGMSWMKQAPGKGLKWMGWINTYSGVPTYADDFKGRFAFSLETSASTAYLQISNLKDEDTARYFCARRDYGSSQWYFDVWSTGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 54 - protein, 213 aa, Artificial Sequence
  Description of Artificial Sequence Synthetic polypeptide
DIVMTQSPKFMSTSVGDRVNITCRASQDVNTAVAWFQQKPGRSPKLLIYSASYRYTGPDHFTGSGSGTDFTFTISSVQAEDLAVYYCQQHYTTPLTFGAGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 55 - protein, 453 aa, Artificial Sequence
  Description of Artificial Sequence Synthetic polypeptide
QVQLQQSGAELVGPGTSVKISCQASGYTFTNYWIGWAKQRPGHGLEWIGDIYPGGGYTNYNKKFKGKATLTADKSSSTAYMQFSSLTSEDSAIYYCSRSRGHGSNFYWYFDVWGTGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 56 - protein, 214 aa, Artificial Sequence
  Description of Artificial Sequence Synthetic polypeptide
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 57 - protein, 446 aa, Artificial Sequence
  Description of Artificial Sequence Synthetic polypeptide
QVQLQQSGAELVTPGASVKLSCKTSGHTFTSYWMNWVNQKPGQGLEWIGWIYGGSGNTKYNQKFQGKATLTVDTSSSTAYMELRSLTSDDSAVYFCASGTNFFDYWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 58 - protein, 237 aa, Mus musculus
MVARLTAFLVCLVFSLATLVQRGYGDTDGFNLEDALKETSSVKQRWDHFSTTTRRPVTTRAPANPAERWDHVATTTTRRPGTTRAPSNPMELDGFDLEDALDDRNDLDGPKKPSAGEAGGWSDKDLEDIVEGGGYKPDKNKGGGGYGSNDDPGSGISTETGTIAGVASALAMALIGAVSSYISYQQKKFCFSIQQGLNADYVKGENLEAVVCEEPQVTYSKQETQSAEPPPPEPPRI

SEQ ID NO: 59 - protein, 262 aa, Homo sapiens
MVAWRSAFLVCLAFSLATLVQRGSGDFDDFNLEDAVKETSSVKQPWDHTTTTTTNRPGTTRAPAKPPGSGLDLADALDDQDDGRRKPGIGGRERWNHVTTTTKRPVTTRAPANTLGNDFDLADALDDRNDRDDGRRKPIAGGGGFSDKDLEDIVGGGEYKPDKGKGDGRYGSNDDPGSGMVAEPGTIAGVASALAMALIGAVSSYISYQQKKFCFSIQQGLNADYVKGENLEAVVCEEPQVKYSTLHTQSAEPPPPPEPARI

SEQ ID NO: 60 - protein, 278 aa, Mus musculus
MWSAQLLSQLLPLWPLLLLSVLPPAQGSSHRSPPAPARPPCVRGGPSAPRHVCVWERAPPPSRSPRVPRSRRQVVPGTAPPATPSGFEEGPPSSQYPWAIVWGPTVSREDGGDPNSVNPGFLPLDYGFAAPHGLATPHPNSDSMRDDGDGLILGETPATLRPFLFGGRGEGVDPQLYVTITISIIIVLVATGIIFKFCWDRSQKRRRPSGQQGALRQEESQQPLTDLSPAGVTVLGAFGDSPTPTPDHEEPRGGPRPGMPQPKGAPAFQLNRIPLVNL

SEQ ID NO: 61 - protein, 302 aa, Mus musculus
MALLISLPGGTPAMAQILLLLSSACLHAGNSERSNRKNGFGVNQPESCSGVQGGSIDIPFSFYFPWKLAKDPQMSIAWRWKDFHGEFIYNSSLPFIHEHFKGRLILNWTQGQTSGVLRILNLKESDQTRYFGRVFLQTTEGIQFWQSIPGTQLNVTNATCTPTTLPSTTAATSAHTQNDITEVKSANIGGLDLQTTVGLATAAAVFLVGVLGLIVFLWWKRRRQGQKTKAEIPAREPLETSEKHESVGHEGQCMDPKENPKDNNIVYASISLSSPTSPGTAPNLPVHGNPQEETVYSIVKAK

SEQ ID NO: 62 - protein, 5 aa, Homo sapiens
  MOD_RES (4)..(4): Any amino acid
PTPXP

SEQ ID NO: 63 - protein, 6 aa, Homo sapiens
  MOD_RES (4)..(5): Any amino acid
PTPXXP

SEQ ID NO: 64 - protein, 6 aa, Homo sapiens
  MOD_RES (2)..(2): Any amino acid
  MOD_RES (5)..(5): Any amino acid
PXTPXP

SEQ ID NO: 65 - protein, 7 aa, Homo sapiens
  MOD_RES (2)..(2): Any amino acid
  MOD_RES (5)..(6): Any amino acid
PXTPXXP

SEQ ID NO: 66 - protein, 7 aa, Herpes simplex virus 1
PATPAPP

SEQ ID NO: 67 - protein, 7 aa, Homo sapiens
PSTPGTP

SEQ ID NO: 68 - protein, 7 aa, Homo sapiens
EPTPAPE

SEQ ID NO: 69 - protein, 7 aa, Homo sapiens
SPTPAPR

SEQ ID NO: 70 - protein, 7 aa, Homo sapiens
PGTPDPS

SEQ ID NO: 71 - protein, 7 aa, Homo sapiens
PATPLPL